(19) United States
(12) Patent Application Publication
(10) Pub. No.: US 2026/0305994 A1
(43) Pub. Date: Oct. 8, 2026

(54) HIGH-VOLUME BODILY FLUID COLLECTION DEVICES AND RELATED SYSTEMS AND METHODS

(71) Applicant: Tasso, Inc., Seattle, WA (US)

(72) Inventors: Jared Andrew Salstrom, Lake Stevens, WA (US); Bryce Petersen, Seattle, WA (US); Geneva Goldwood, Tacoma, WA (US); Erwin Berthier, Seattle, WA (US); Sebastian Crisologo Bibat, Seattle, WA (US); Michael Lin, Seattle, WA (US); Taylor N. Tyson, Redmond, WA (US); David Perez, Tukwila, WA (US)

(21) Appl. No.: 19/640,373

(22) Filed: Apr. 6, 2026

Related U.S. Application Data
(60) Provisional application No. 63/783,837, filed on Apr. 4, 2025.
(60) Provisional application No. 63/883,554, filed on Sep. 17, 2025.
(60) Provisional application No. 63/934,558, filed on Dec. 9, 2025.

(51) Int. Cl. A61B 5/15; A61B 5/151

(52) U.S. Cl. CPC A61B 5/150099; A61B 5/150221; A61B 5/150343; A61B 5/150442; A61B 5/15113

(57) ABSTRACT

Disclosed herein is a device for withdrawing bodily fluid from a subject. The device includes a housing, a flexible membrane, a floating island, a skin-piercing assembly, and an actuator. The housing has a surface positioned against skin of the subject and an opening extending through the surface. The flexible membrane is stretched across the opening. The floating island is carried by the flexible membrane. The skin-piercing assembly is positioned at least partially within the housing and has a blade configured to pierce the skin of the subject. The actuator is operably coupled to the skin-piercing assembly and is movable relative to the housing to deploy the skin-piercing assembly through the opening and into the skin of the subject. Movement of the skin-piercing assembly away from the opening draws a vacuum within the housing, causing the skin of the patient to be drawn through the opening and assume a two-tiered shape.

### CROSS-REFERENCE TO RELATED APPLICATIONS

This application claims the benefit of (i) U.S. Provisional Patent Application No. 63/934,558, filed Dec. 9, 2025, and titled “HIGH-VOLUME BODILY FLUID COLLECTION DEVICES AND RELATED SYSTEMS AND METHODS,” (ii) U.S. Provisional Patent Application No. 63/883,554, filed Sep. 17, 2025, and titled “HIGH-VOLUME BODILY FLUID COLLECTION DEVICES AND RELATED SYSTEMS,” and (iii) U.S. Provisional Patent Application No. 63/783,837, filed Apr. 4, 2025, and titled “HIGH-VOLUME BODILY FLUID COLLECTION DEVICES AND RELATED SYSTEMS,” which are incorporated herein by reference in their entireties.

### TECHNICAL FIELD

The present technology is related to collecting bodily fluid from a subject and, in particular, to methods, systems, and apparatuses for increasing the collection volume of bodily fluids via handheld bodily fluid collection devices.

### BACKGROUND

Devices, systems, and methods to collect bodily fluids, such as blood, are widely used in personalized, clinical, and field medical applications. Biological samples are commonly collected using simple lancing devices or more sophisticated devices that require trained personnel (e.g., phlebotomy venipunctures). Transferring bodily fluids to a container, receptacle, or analysis device often requires several steps, which can be time-consuming, prone to error, and/or cumbersome. Moreover, collecting a sufficient volume of bodily fluids can be difficult, particularly for untrained users employing many existing personalized devices.

### BRIEF DESCRIPTION OF THE DRAWINGS

Many aspects of the present technology can be better understood with reference to the following drawings. The components in the drawings are not necessarily to scale. Instead, emphasis is placed on illustrating clearly the principles of the present disclosure.

FIGS. 1A-1C are perspective views of a bodily fluid collection device configured in accordance with embodiments of the present technology. FIG. 1A is a perspective view of the bodily fluid collection device configured in accordance with embodiments of the present technology. FIG. 1B is a perspective view of the bodily fluid collection device of FIG. 1A in use by the patient configured in accordance with embodiments of the present technology. FIG. 1C is a perspective view illustrating the detachment of the collection reservoir from the bodily fluid collection device of FIGS. 1A and 1B configured in accordance with embodiments of the present technology.

FIG. 2 is a cross-sectional side view of the bodily fluid collection device of FIG. 1A configured in accordance with embodiments of the present technology.

FIG. 3 is a schematic cross-sectional top view of the bodily fluid collection device of FIG. 1A configured in accordance with embodiments of the present technology.

FIGS. 4A-4D are schematic perspective top views of floating islands of the bodily fluid collection device of FIG. 1A configured in accordance with embodiments of the present technology.

FIGS. 5A-5C are schematic perspective top views of housings of the bodily fluid collection device of FIG. 1A configured in accordance with embodiments of the present technology.

FIGS. 6A-6C are schematic perspective top views of the housings of FIGS. 5A-5C, respectively, without the floating islands configured in accordance with embodiments of the present technology.

FIGS. 6D and 6E are schematic perspective top views of the housings of the bodily fluid collection device of FIG. 1A configured in accordance with embodiments of the present technology.

FIGS. 7A-7C are schematic perspective top views of the floating islands of the bodily fluid collection device of FIG. 1A configured in accordance with embodiments of the present technology. FIG. 7D is a perspective top view of the floating island of the bodily fluid collection device of FIG. 1A configured in accordance with embodiments of the present technology.

FIGS. 8A and 8B are schematic cross-sectional side views of the bodily fluid collection device of FIG. 1A configured in accordance with embodiments of the present technology.

FIGS. 9A-9J are schematic top views of cuts created by the bodily fluid collection device of FIG. 1A configured in accordance with embodiments of the present technology.

FIGS. 10A and 10B are cross-sectional side views of the housing of FIG. 2 in a first position and a second position, respectively, configured in accordance with embodiments of the present technology.

FIG. 11 is a schematic cross-sectional side view of a bodily fluid collection device configured in accordance with embodiments of the present technology.

FIG. 12A is a perspective view of a bodily fluid collection device in a first position in accordance with embodiments of the present technology. FIGS. 12B and 12C are perspective cross-sectional views of the bodily fluid collection device of FIG. 12A in a second position and a third position, respectively, configured in accordance with embodiments of the present technology.

FIGS. 13A-13C are perspective views of the skin-piercing assembly of FIGS. 12B and 12C in a first position, second position, and third position, respectively, configured in accordance with embodiments of the present technology.

FIG. 14A is a perspective view of a bodily fluid collection device configured in accordance with embodiments of the present technology. FIG. 14B is a partially cross-sectional perspective view of the bodily fluid collection device of FIG. 14A configured in accordance with embodiments of the present technology.

FIG. 15A is a transparent perspective view of a bodily fluid collection device in a first position in accordance with additional embodiments of the present technology. FIGS. 15B and 15C are transparent perspective views of the bodily fluid collection device of FIG. 15A in a second position in accordance with additional embodiments of the present technology.

FIGS. 16A-16C are cross-sectional side views of a bodily fluid collection device in a first position, second position, and third position, respectively, configured in accordance with embodiments of the present technology.

FIG. 17A is a side view of a bodily fluid collection device in a first position configured in accordance with embodiments of the present technology. FIG. 17B is a cross-sectional side view of the bodily fluid collection device of FIG. 17A in the first position configured in accordance with embodiments of the present technology. FIG. 17C is a side view of the bodily fluid collection device of FIG. 17A in a second position configured in accordance with embodiments of the present technology. FIG. 17D is a side view of the bodily fluid collection device of FIG. 17A in a third position configured in accordance with embodiments of the present technology. FIG. 17E is a cross-sectional side view of the bodily fluid collection device of FIG. 17A in the third position configured in accordance with embodiments of the present technology.

FIG. 18 is a cross-sectional side view of a bodily fluid collection device configured in accordance with additional embodiments of the present technology.

FIGS. 19A and 19B are partially transparent perspective views of a bodily fluid collection device in a first position and a second position, respectively, configured in accordance with additional embodiments of the present technology.

FIGS. 20A-20C are partially cross-sectional perspective views of the bodily fluid collection device in a first position, a second position, and a third position, respectively, configured in accordance with embodiments of the present technology.

FIG. 21A is a partially cross-sectional perspective view of the bodily fluid collection device configured in accordance with embodiments of the present technology.

FIG. 21B is a perspective view of the floating island of the bodily fluid collection device of FIG. 21A configured in accordance with embodiments of the present technology.

FIG. 22A is a partially transparent side view of the skin-piercing assembly of FIGS. 13A-13C configured in accordance with embodiments of the present technology.

FIG. 22B is a bottom view of the skin-piercing assembly of FIG. 22B configured in accordance with embodiments of the present technology.

FIG. 23 is a cross-sectional side view of the skin-piercing assembly of FIG. 2 configured in accordance with embodiments of the present technology.

FIGS. 24A-24D are charts of data comparing configurations of the bodily fluid collection devices of FIGS. 4A-6B, 6D, and 6E configured in accordance with embodiments of the present technology. FIG. 24A is a modified whisker plot of the bodily fluid flow rates of the devices of FIGS. 6A, 6B, 6D, and 6E. FIG. 24B is a modified whisker plot of the bodily fluid flow rates of the devices of FIGS. 5A-5C and additional devices. FIG. 24C is a modified whisker plot of the bodily fluid flow rates of the devices of FIGS. 4A-4D. FIG. 24D is a modified whisker plot of the bodily fluid flow rates of a variety of cut configurations.

FIG. 25 is a perspective view of a bodily fluid collection device in a first position in accordance with additional embodiments of the present technology.

FIGS. 26A and 26B are a cross-sectional side view and a perspective cross-sectional side view, respectively, of the device of FIG. 25 in the first position in accordance with embodiments of the present technology.

FIGS. 26C and 26D are a cross-sectional side view and a perspective cross-sectional side view, respectively, of the device of FIG. 25 in a second in accordance with embodiments of the present technology.

FIGS. 26E and 26F are a cross-sectional side view and a perspective cross-sectional side view, respectively, of the bodily fluid collection device of FIG. 25 in a third position in accordance with embodiments of the present technology.

FIGS. 27A and 27B are a front perspective view and a back perspective view, respectively, of a skin-piercing assembly and an island member of the device of FIGS. 25-26F in accordance with embodiments of the present technology.

FIG. 28 is a partially-transparent side view of the skin-piercing assembly of FIGS. 27A and 27B in accordance with embodiments of the present technology.

FIGS. 29 and 30 are perspective views of a slot and a post and pin of the device of FIGS. 25-26F in accordance with embodiments of the present technology.

FIG. 31 is a top view of a base, a membrane, and an island member of the device of FIGS. 25-26F in accordance with embodiments of the present technology.

FIG. 32 is a perspective of a lockout mechanism of the device 2500 of FIGS. 25-26F in accordance with embodiments of the present technology.

FIG. 33 is a partially exploded perspective view of a bodily fluid collection device in accordance with additional embodiments of the present technology.

FIG. 34A is a side view of the base of the bodily fluid collection device of FIG. 33 in accordance with embodiments of the present technology.

FIG. 34B is a side view of the housing of the bodily fluid collection device of FIG. 33 in accordance with embodiments of the present technology.

FIGS. 35A-35C are perspective views of a bodily fluid collection device in a first position, a second position, and a third position, respectively, in accordance with embodiments of the present technology.

FIGS. 36A-36C are cross-sectional perspective views of the bodily fluid collection device of FIGS. 35A-35C in the first position, the second position, and the third position, respectively, in accordance with embodiments of the present technology.

FIG. 37 is a perspective view of the base of the bodily fluid collection device of FIG. 35A in accordance with additional embodiments of the present technology.

FIG. 38 is a perspective view of a portion of the housing of the bodily fluid collection device of FIG. 35A in accordance with embodiments of the present technology.

FIGS. 39A-39C are perspective, partially transparent views of the bodily fluid collection device of FIGS. 35A-35C in a first position, a second position, and a third position, respectively, in accordance with embodiments of the present technology.

### DETAILED DESCRIPTION

### I. INTRODUCTION

The present technology is directed generally to devices and methods for collecting a high volume of bodily fluid, such as blood, from a subject. A handheld bodily fluid collection device configured in accordance with the present technology can include a housing having an opening, a skin-piercing assembly positioned at least partially within the housing, and an actuator coupled to the skin-piercing assembly. The actuator can be actuated to cause a blade of the skin-piercing assembly to form an incision (e.g., cut, abrasion, wound) in the skin of a patient. In some embodiments, the devices and methods disclosed herein use a vacuum source configured to dynamically generate a vacuum that is applied to the patient's skin to facilitate the collection of the bodily fluid. In some embodiments, the handheld bodily fluid collection devices disclosed herein include a surface that contacts the skin of the subject and is shaped to define an opening into which a portion of the subject's skin is drawn (e.g., via vacuum pressure). In some embodiments, the surface is flat or substantially flat with respect to a plane of the subject's skin (e.g., a plane of the surface is approximately parallel to the skin). When used, the device can be placed on the skin so that a collection reservoir of the device is perpendicular to the ground. In some aspects of the present technology, this orientation allows blood to easily flow from the housing to a collection reservoir with the aid of gravity.

In some embodiments, the bodily fluid collection device includes an outer housing and an inner housing that houses portions of an actuator, a skin-piercing assembly, a membrane, and a floating island disposed on the membrane. When the device is actuated, the membrane, floating island, and skin of the patient can be drawn up into the device to create a two-tiered shape. In some aspects of the present technology, separating the majority of the functional components of the device from an outer housing makes the device less prone to mechanical failure. For example, the outer housing can be ergonomically shaped while the inner housing can be functionally shaped. Further, if the device is dropped, damage will be more likely to occur to the outer housing than the inner housing. In other aspects of the present technology, the two-tiered shape of the membrane increases the bodily fluid flow rates of the device. The inner housing can be wider at the bottom than it is at the top. The outer housing can have a diameter, and the inner housing can have a vacuum diameter. In some aspects of the present technology, the variable diameter of the inner housing can allow the vacuum diameter to be increased without also increasing the diameter of the outer housing. More specifically, the diameter of the upper portion of the inner housing remains the same so the diameter of the upper portion of the outer housing remains the same. Thus, this configuration can maintain a desirable form factor of the device while increasing bodily fluid flows.

In some embodiments, the membrane can include tacky surfaces and/or adhesive material on both the inside and outside of the membrane. In some aspects of the present technology, the tackiness of the external surface can facilitate adhering the device to the skin of the patient, enabling a vacuum to be applied to portions of the skin within the footprint of the housing and inhibiting unintentional leaks.

In some embodiments, the bodily fluid collection device includes an outflow port for blood flowing from a wound caused by the skin-piercing assembly. The outflow port can be coupled to the housing at an angle relative to the skin, such as 10 degrees. In some aspects of the present technology, the angle of the outflow port can enhance the flow of blood between the skin and the collection reservoir, which can reduce the amount of time the collected material (e.g., blood) spends in contact with plastic components of the housing that could degrade the quality of the collected material. Coupling the outflow port at an angle can also speed up blood collection, which can be more comfortable for the patient. In other aspects of the present technology, coupling the outflow port at an angle reduces a lip between the outflow port and other components of the housing to improve bodily fluid flow to the outflow port, inhibiting build-up on other components of the housing and/or overflow of the other components that could lead to degradation of the sample.

In some embodiments, the floating island can include channel walls that surround a fluid outflow channel, and the channel walls can include flanges configured to help direct bodily fluid flow to an outflow port of the housing. In some aspects of the present technology, the flanges can direct fluid toward the outflow port when there is a large stream of fluid from the wound. In other aspects of the present technology, the flanges direct fluid toward the outflow port and inhibit fluid from spreading on the membrane and/or flowing to the sides of the outflow port.

In some embodiments, the housing also includes a vent and portions of the actuator can be fluidly disconnected from portions of the housing. In some aspects of the present technology, the distinct portions can be used to control the amount of air that is forced out of the vent and, consequently, the degree of negative pressure that is generated when the actuator is actuated.

In some embodiments, the membrane isn't exposed to flowing fluid between the floating island and a hole of the housing (e.g., an exposed portion of the membrane does not exist). More specifically, a vacuum diameter of the housing and an island outer diameter of the housing can be the same. In some aspects of the present technology, reducing the exposed portion of the membrane (e.g., the portion blood can contact) can decrease the degradation of collected blood by limiting exposure to the tacky material on the inner surface of the membrane.

In some embodiments, the floating island is half-moon shape. In some aspects of the present technology, this configuration can reduce trauma to the patient's skin by allowing the vacuum pressure applied to the top half of the skin to be applied in a uniform manner while maintaining benefits to blood flow generated by the floating island.

In some embodiments, the floating island can have an island outer diameter and an island inner diameter. The housing can have a vacuum diameter. In some aspects of the present technology, increasing the island outer diameter increases flow rates, decreasing the island inner diameter increases flow rates, and increasing the vacuum diameter increases bodily fluid flow rates. Further, a device with a floating island generally has higher flow rates than a device without a floating island.

In some embodiments, the floating island can have only circular walls, a flat portion, and notches. The flat portion can further define a cutout portion. More specifically, the floating island does not have channel walls to help direct bodily fluid flow. In some aspects of the present technology, this configuration creates a laminar flow of fluid into the outflow port.

In some embodiments, the floating island can include channel walls and flanges configured to direct bodily fluid flow in the housing. In some aspects of the present technology, the channel walls and the flanges create guides for the bodily fluid flowing towards the outflow port.

In some embodiments, the floating island can include a stepped portion overmolded with a flexible elastomer (e.g., flexible plastic, TPU, Santoprene). In some aspects of the present technology, the flexible elastomer can reduce bruising when vacuum pressure is applied to the skin by creating a softer contact surface between the floating island and the skin. In some embodiments, the edges (e.g., corners, circumference) of the stepped portion can be rounded or chamfered to further reduce the risk of bruising.

In some embodiments, compressive force can be applied to the skin rather than vacuum pressure via biasing members directly coupling the actuator to the floating island. In some aspects of the present technology, compressive forces can create similar benefits to the vacuum pressure and can be easier to generate because they obviate the vacuum source.

In some embodiments, the housing can include retention arms configured to hold a biasing member. The biasing member can be configured to bias the actuator in a retraction direction. In some aspects of the present technology, the retention arms inhibit the floating island from becoming askew, which could inhibit blood flow toward the outflow port.

In some embodiments, the actuator can start almost fully compressed, such that a patient only needs to apply a small amount of force to the actuator to deploy the skin-piercing device. In some aspects of the present technology, the reduced force required for activation of the device can be beneficial for patients with mobility and strength issues. It can also reduce the form factor of the housing prior to deployment, which can decrease packaging costs. Further, this configuration allows the vent to be removed, and omitting the vent can reduce manufacturing costs. In some embodiments, the actuator and/or housing can include additional features designed to inhibit accidental actuation of the device.

In some embodiments, the outflow port is composed of a flexible material, such as rubber. In some aspects of the present technology, this can allow the outflow port to be stretched or compressed, and thus coupled to a variety of collection reservoirs. More specifically, the outflow port can be coupled to collection reservoirs with openings of different sizes.

In some embodiments, the actuator can be offset from a center of the housing such that the actuator can be actuated along an axis angularly offset from a latitudinal axis of the housing. In some aspects of the present technology, placing the actuator offset from the latitudinal axis of the housing can enhance the ergonomics of the device, which can be beneficial when the device is used by disabled users or users with limited mobility.

In some embodiments, the skin-piercing assembly can include skin guards that shape the skin prior to deployment of the blade into the skin. In some aspects of the present technology, the skin guards can help shape the skin prior to deployment of the blade to ensure a proper wound shape.

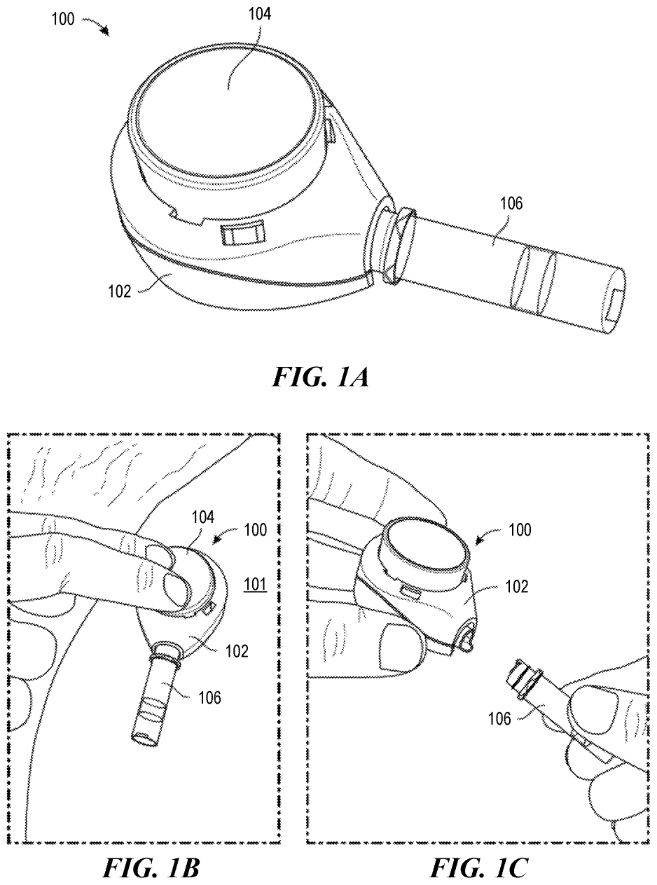

Certain details are set forth in the following description and in FIGS. 1A-32 to provide a thorough understanding of various embodiments of the present technology. In other instances, well-known structures, materials, operations, and/or systems often associated with bodily fluid collection devices, and/or the like, are not shown or described in detail in the following disclosure to avoid unnecessarily obscuring the description of the various embodiments of the technology. Those of ordinary skill in the art will recognize, however, that the present technology can be practiced without one or more of the details set forth herein, and/or with other structures, methods, components, and so forth. The terminology used below is to be interpreted in its broadest reasonable manner, even though it is being used in conjunction with a detailed description of certain examples of embodiments of the technology. Indeed, certain terms may even be emphasized below; however, any terminology intended to be interpreted in any restricted manner will be overtly and specifically defined as such in this Detailed Description section.

The accompanying Figures depict embodiments of the present technology and are not intended to be limiting of its scope unless expressly indicated. The sizes of various depicted elements are not necessarily drawn to scale, and these various elements may be enlarged to improve legibility. Component details may be abstracted in the Figures to exclude details such as the position of components and certain precise connections between such components when such details are unnecessary for a complete understanding of how to make and use the present technology. Many of the details, dimensions, angles, and other features shown in the Figures are merely illustrative of particular embodiments of the disclosure. Accordingly, other embodiments can have other details, dimensions, angles, and features without departing from the present technology. In addition, those of ordinary skill in the art will appreciate that further embodiments of the present technology can be practiced without several of the details described below. With regard to the terms “distal” and “proximal” within this description, unless otherwise specified, the terms can reference a relative position of the portions of a bodily fluid collection device with reference to an operator and/or a location on the patient's skin. As used herein, the designations “rearward,” “forward,” “upward,” “downward,” “bottom,” “top,” “lower,” “upper,” and the like are not meant to limit the referenced component to a specific orientation. It will be appreciated that such designations refer to the orientation of the referenced component as illustrated in the Figures; the systems of the present technology can be used in any orientation suitable to the user.

In the Figures, identical reference numbers identify identical, or at least generally similar, elements. To facilitate the discussion of any particular element, the most significant digit or digits of any reference number refer to the Figure in which that element is first introduced. For example, housing 102 is first introduced and discussed with reference to FIG. 1. The headings provided herein are for convenience only and should not be construed as limiting the subject matter disclosed.

To the extent any materials incorporated herein by reference conflict with the present disclosure, the present disclosure controls.

### II. SELECT EMBODIMENTS OF BODILY FLUID COLLECTION DEVICES

Typically, blood for blood tests is withdrawn from a patient by collecting blood directly from a vein of the patient (e.g., via a venipuncture). However, blood for blood tests often needs to be withdrawn outside of a medical setting and without the aid of a medical professional. Personalized, handheld blood collection devices can be used to obtain such blood, but there can be challenges with collecting a sufficient volume of blood using such devices, particularly as employed by laypersons. To make blood collection accessible to a wide variety of people, bodily fluid collection devices (e.g., device 100 of FIGS. 1A-1C) can employ various technologies that simplify blood collection and increase the volume of blood collected.

For example, as disclosed herein, bodily fluid collection devices can employ vacuum pressure to help draw blood from the incision site into the collection device (e.g., collection reservoir 106 of FIGS. 1A-1C). In particular, the vacuum pressure can increase capillary action within the skin and increase the amount of blood withdrawn/collected. Thus, as demonstrated in the embodiments disclosed herein, bodily fluid collection devices can be configured to engage additional skin area at and/or adjacent to the incision size, and thereby optimize blood volume collection.

The portion of the skin in the accommodating space is pierced by a piercing element (e.g., a blade, a needle, etc.) positioned in the housing of the bodily fluid collection device. The piercing element moves through an opening (e.g., a hole) to pierce the skin. Blood is drawn through the opening (e.g., via vacuum pressure and/or fluidic channels) and collected by the collection device. In some embodiments, the bodily fluid collection device includes a single opening through which skin is drawn by vacuum and through which a piercing element moves to pierce the skin (e.g., the lower portion/surface of a housing defines one opening into which the subject's skin is drawn and where the skin is pierced).

FIGS. 1A-1C are perspective views of a bodily fluid collection device 100 (“device 100”) configured in accordance with embodiments of the present technology. FIG. 1A is a perspective view of the bodily fluid collection device 100 configured in accordance with embodiments of the present technology. The device 100 can be handheld with a size that is easily grasped and manipulated by one or both of a patient's hands. Such handheld devices advantageously allow a patient to collect a bodily fluid sample (e.g., a blood sample) without assistance from another individual. In some embodiments, the handheld devices of the present technology can be operated by a layperson outside of a medical setting (e.g., at home or in a field clinic) and without the aid of a medical professional.

In the illustrated embodiment, the device 100 includes a housing 102 and an actuator 104. The actuator 104 (e.g., a button, a switch) can be movable relative to the housing 102 to actuate/initiate withdrawal of a bodily fluid (e.g., blood) from the patient. The housing 102 is removably coupled to a collection reservoir 106 (e.g., a tube, a collection device, a transfer device, a cartridge, and/or the like) for receiving the bodily fluid withdrawn from the patient. The collection reservoir 106 can act as a removable and standardized container for bodily fluid that can be detached and used in clinical and laboratory equipment or workflows (e.g., for diagnostics and/or biomarker detection).

FIG. 1B is a perspective view of the bodily fluid collection device 100 of FIG. 1A in use by the patient configured in accordance with embodiments of the present technology. To collect a bodily fluid sample, the device 100 is applied to a patient's body, with a bottom surface of the housing 102 positioned against the skin 101 of the patient and the actuator 104 positioned away from the skin 101. Actuating (e.g., pressing, twisting, pulling) the actuator 104 deploys a skin-piercing feature (e.g., a blade, lancet) from within the device 100 to pierce the skin 101. Subsequent retraction of the actuator 104 away from the skin generates a vacuum within the device 100 that acts against the patient's skin either directly or indirectly, and before and/or after deployment of the skin-piercing feature. Bodily fluid from the resulting incision is withdrawn into the housing 102 and collected into the collection reservoir 106.

In the illustrated embodiment, the device 100 is oriented such that the collection reservoir 106 is generally perpendicular to the ground (e.g., perpendicular to a surface the patient is standing or sitting on). In some aspects of the present technology, this orientation allows blood to easily flow from the housing 102 to the collection reservoir 106 with the aid of gravity. In other embodiments, the collection reservoir 106 can be oriented at other angles, such as 75 degrees relative to the ground.

FIG. 1C is a perspective view illustrating detachment of the collection reservoir 106 from the bodily fluid collection device 100 of FIGS. 1A and 1B configured in accordance with embodiments of the present technology. Once a desired amount of bodily fluid has been collected into the collection reservoir 106, the device 100 is removed from the skin 101, and the collection reservoir 106 is detached from the housing 102. In some embodiments, the device 100 can include one or more features that are generally similar to or identical to the bodily fluid collection devices disclosed in can include some features generally similar or identical to, and can operate in a manner generally similar or identical, any of the skin-piercing assemblies described in (i) U.S. patent application Ser. No. 17/006,248, titled “BODILY FLUID COLLECTION DEVICES AND RELATED METHODS,” and filed Aug. 28, 2020; (ii) U.S. Pat. No. 11,633,136, titled “BODILY FLUID COLLECTION DEVICES AND RELATED METHODS,” and filed Feb. 25, 2022; and/or (iii) U.S. Nonprovisional patent application Ser. No. 19/331,907, titled “METHODS, SYSTEMS, AND APPARATUSES FOR COLLECTING A HIGH VOLUME OF BODILY FLUID USING HANDHELD BODILY FLUID COLLECTION DEVICES,” filed Sep. 17, 2025, each of which is incorporated herein by reference in its entirety.

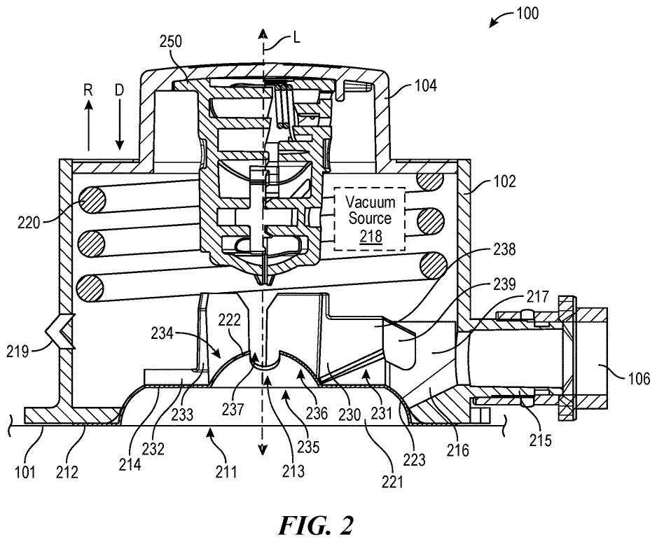

FIG. 2 is a cross-sectional side view of the bodily fluid collection device 100 of FIG. 1A configured in accordance with embodiments of the present technology. In the illustrated embodiment, the housing 102 (FIGS. 1A-1C) is a circular extrusion, while in other embodiments, the housing 102 is ergonomically designed for a patient to hold (e.g., as shown in FIGS. 1A-1C). Any number of elements of the device 100 can be housed partially or entirely within the housing 102. In some embodiments, the housing 102 is an inner housing and can be positioned within an outer housing not shown. In such embodiments, separating the majority of the functional components of the device 100 in an inner housing makes the housing 102 less prone to mechanical failure. Simplifying the housing to have a cylindrical rather than ergonomic shape can allow for easier mechanical movements (e.g., linear, rotational) than if the movements were occurring in a variable-shaped housing. In some embodiments, one or more components of the device 100 can be made of single-use plastic so the device 100 can be easily disposed of.

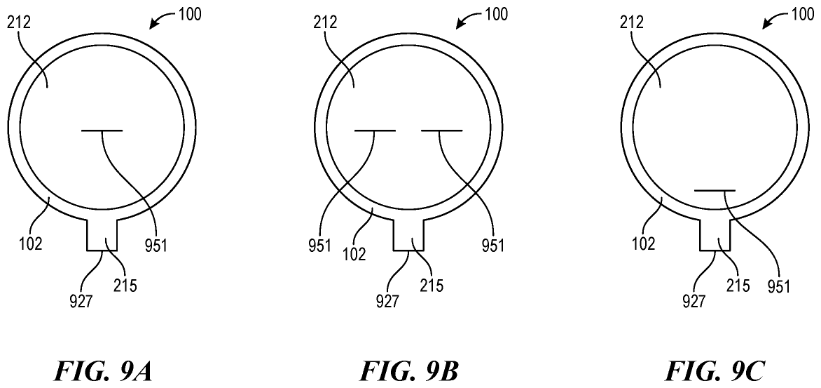

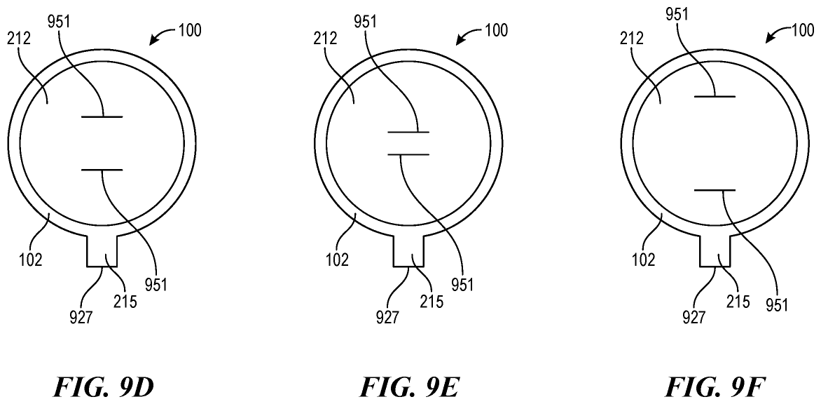

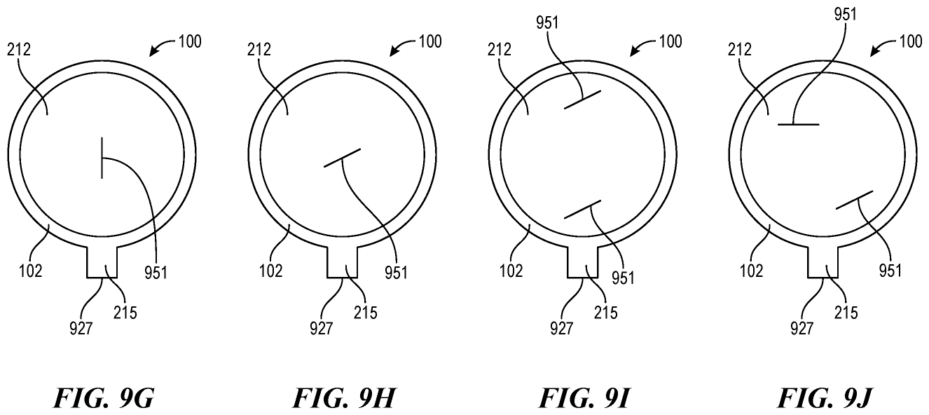

In the illustrated embodiment, the housing 102 and other components of the device 100 are generally centered on a central axis L (e.g., perimeters are generally equally distanced circumferentially from the axis L). In other embodiments, some or all of the components can be offset from the axis L. As shown in FIGS. 1A-1C, a portion of the actuator 104 can extend past the housing 102 while the housing 102 is obscured. With reference to FIG. 2, the actuator 104 is operably coupled to a skin-piercing assembly 250 configured to move within the housing 102 in both a deployment direction D (toward the skin 101 of the patient) and a retraction direction R (away from the skin 101), as described in further detail below. The skin-piercing assembly 250 can include one or more cutting edges configured to pierce (e.g., cut, incise) the skin 101 when deployed. These cutting edges can be oriented in a variety of positions, as described in greater detail below with reference to FIGS. 9A-9J. The skin-piercing assembly 250 can include some features generally similar or identical to, and can operate in a manner generally similar or identical to, any of the skin-piercing assemblies described in (i) U.S. patent application Ser. No. 17/006,248, titled “BODILY FLUID COLLECTION DEVICES AND RELATED METHODS,” and filed Aug. 28, 2020, and/or (ii) U.S. Pat. No. 11,633,136, titled “BODILY FLUID COLLECTION DEVICES AND RELATED METHODS,” and filed Feb. 25, 2022, each of which is incorporated by reference herein in its entirety.

In the illustrated embodiment, the secondary housing 210 defines an opening 211 at the bottom of the secondary housing 210. The opening 211 is circular. In other embodiments, the opening 211 can be square, ovular, rectangular, or the like. In the illustrated embodiment, a membrane 212 extends across at least a portion of the opening 211 (e.g., stretched across, creating a drum-tight covering of the opening 211) on the outside of the secondary housing 210. The membrane 212 can include some features generally similar or identical to, and can operate in a manner generally similar or identical to, any of the membranes described in U.S. Pat. No. 11,510,659, titled “BODILY FLUID COLLECTION DEVICES AND RELATED METHODS,” and filed Feb. 26, 2021, which is incorporated herein by reference in its entirety. In some embodiments, the edges of the opening 211 can be chamfered or rounded to reduce the risk of damaging the membrane 212. In the illustrated embodiment, the membrane 212 is flexible and includes tacky substances and/or textures that create gripping surfaces (e.g., sticky surfaces) on internal and external surfaces of the membrane 212. The gripping surfaces can be sufficiently sticky to adhere the bottom of the device 100 to the skin 101 while allowing a user to remove the device 100 without traumatizing the skin 101. In some aspects of the present technology, the tackiness of the external surface can facilitate the device 100 adhering to the skin 101 of the patient, enabling a vacuum to be applied to portions of the skin 101 within the footprint of the housing 102 and inhibiting unintentional leaks. In the illustrated embodiments, the membrane 212 includes a precut hole 213 at approximately the intersection of axis L with the membrane 212 that aligns with a cutting edge (e.g., a blade) of the skin-piercing assembly 250. For example, when the skin-piercing assembly 250 is deployed, the cutting edge will pass through the precut hole in the membrane 212 without contacting a surface of the membrane 212. In some embodiments, the precut hole 213 is omitted and the cutting edge of the skin-piercing assembly 250 pierces the membrane 212 when it is deployed.

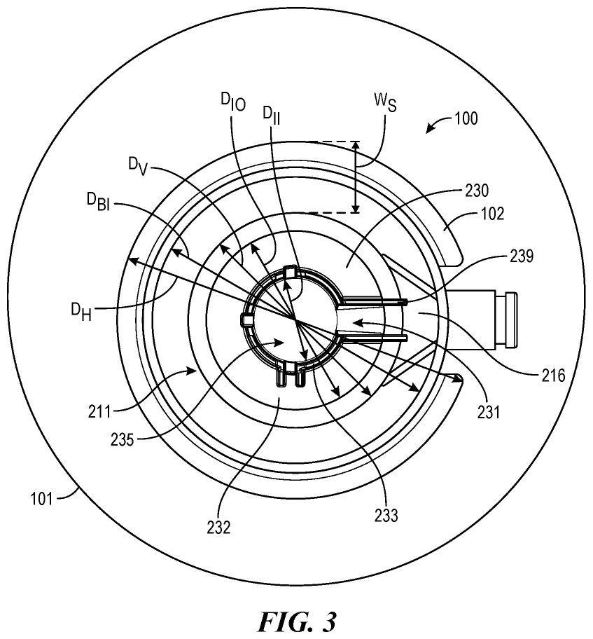

In the illustrated embodiment, the internal tacky surface of the membrane 212 affixes a floating island 230 (e.g., an island member, a skin-shaping member) to the membrane 212. The floating island 230 is affixed to the membrane 212 such that the floating island 230 is spaced apart from the bottom inner surface of the housing 102 (e.g., there is at least a width of the membrane 212 separating the floating island 230 and an edge of the opening 211). The floating island includes a flat portion 232. As best seen in FIG. 3 described in detail below, the flat portion 232 extends circumferentially around circular walls 233 of the floating island 230. With reference to FIG. 2, the circular walls 233 define a lumen 234 and a cutting region 235 at a proximal end 236 of the lumen 234. The cutting edge of the skin-piercing assembly 250 can cut the skin 101 of the patient at the cutting region 235. For example, when deployed, the skin-piercing assembly 250 can move towards the cutting region 235 and into the lumen 234 to create one or more cuts (FIGS. 9A-9J) to the skin 101. The circular walls 233 can further define notches 237 that can be configured to allow protruding portions of the skin-piercing assembly 250 to easily move through the lumen 234. The notches 237 can also be configured to allow the cutting edge(s) of the skin-piercing assembly 250 to be positioned (e.g., oriented, located) at various locations within the cutting region 235 to create a variety of cut patterns, as described in greater detail below with reference to FIGS. 9A-9J.

In some embodiments, the notches 237 include features that correspond to features of the skin-piercing assembly 250 such that when the features of the skin-piercing assembly 250 contact the features of the notches 237, the skin-piercing assembly 250 is activated (e.g., deployed). In some embodiments, the circular walls 233 are omitted and the flat portion 232 extends circumferentially around the cutting region 235. In the illustrated embodiment, the floating island 230 is made of a stiff material (e.g., hardened plastic) such that the flat portion 232 of the floating island 230 maintains a corresponding flat portion 214 of the membrane 212 when vacuum is applied to the membrane 212. As such, the membrane 212 generally has a two-tiered shape when a vacuum is present in the housing 102, with a first tier 221 between the patient's skin 101 and the flat portion 232 and a second tier 222 extending into the proximal end 236 of the lumen 234.

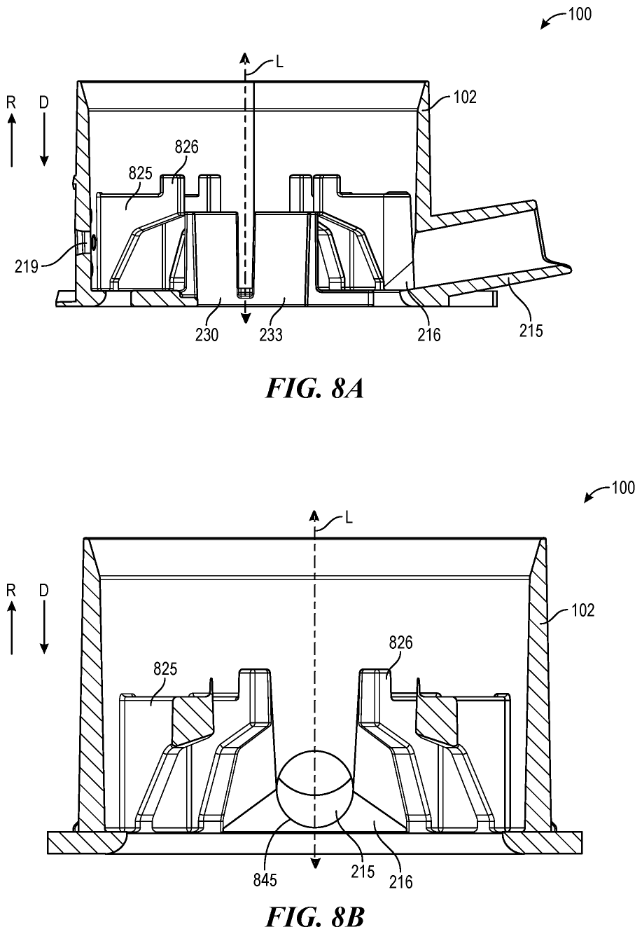

The housing 102 further includes an outflow port 215 that can be removably coupled to the collection reservoir 106. In the illustrated embodiment, the outflow port 215 is parallel to the skin 101 and has an elongated tubular form factor. In other embodiments, the outflow port 215 can be at an angle relative to the skin 101 and/or have a non-circular cross-section (e.g., oval, square, rectangle). For example, the outflow port 215 can be at approximately a 5-degree, 6-degree, 7-degree, 8-degree, 9-degree, 10-degree, 11-degree, 12-degree, 13-degree, 14-degree, 15-degree, or any degree therebetween angle away from the skin 101. In some aspects of the present technology, the angle of the outflow port 215 can enhance the flow of blood between the skin 101 and the collection reservoir 106, which can reduce the amount of time the collected material (e.g., blood) spends in contact with components of the housing 102 that could degrade the quality of the collected material. In some embodiments, the outflow port 215 includes features along the external surface of the outflow port 215 configured to enhance the connection between the outside of the outflow port 215 and the inside of the collection reservoir 106. For example, the outflow port 215 can include protrusions, and the collection reservoir 106 can include corresponding recesses. In the illustrated embodiment, adjacent to the outflow port 215 is a ramp portion 216 that facilitates the movement of blood from the floating island 230 to the outflow port 215, as discussed in greater detail below with reference to FIGS. 8A and 8B. In some embodiments, the housing 102 includes ramp walls 217 positioned from the inside of the housing 102 to the edge of the opening 211 on both sides of the ramp portion 216. The ramp walls 217 can be configured to inhibit fluid from spreading beyond the footprint of the ramp portion 216 and/or direct fluid towards the outflow port 215.

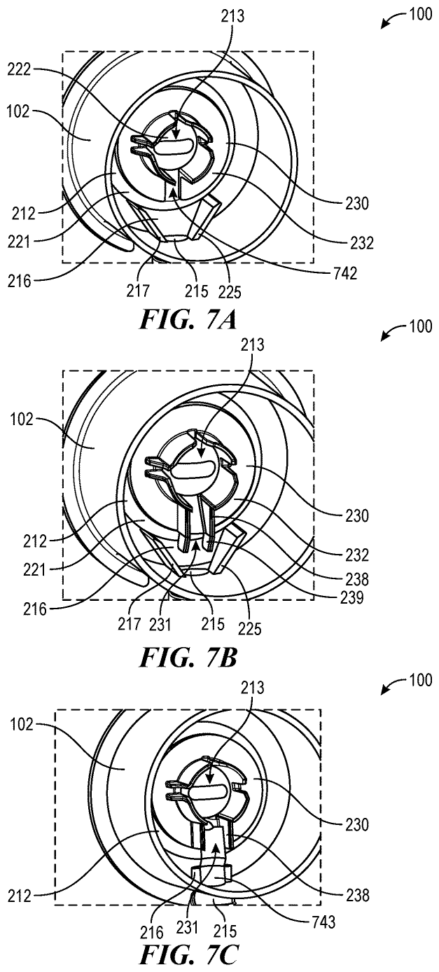

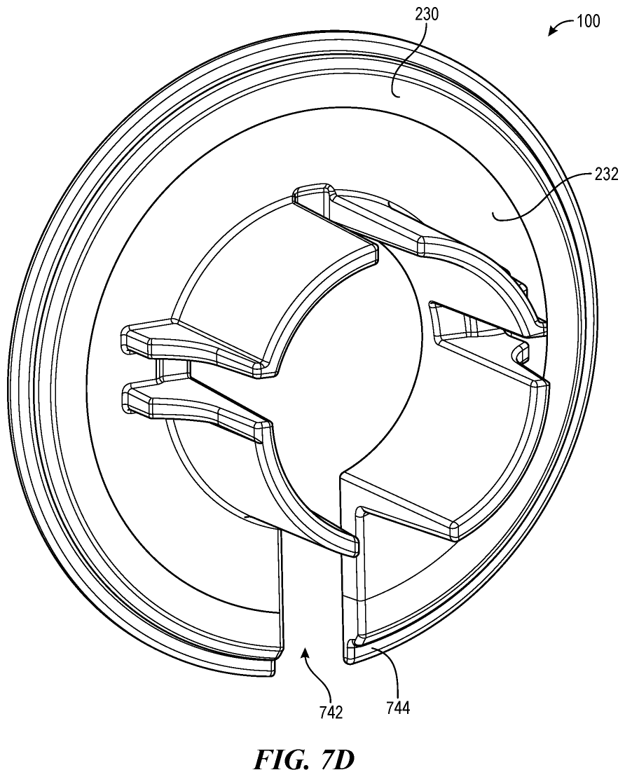

As described in greater detail below with reference to FIGS. 7A-7C, the floating island 230 includes channel walls 238 that define an outflow channel 231 configured to allow fluid to and/or direct fluid to flow out of the floating island 230 towards the collection reservoir 106. With reference to FIG. 2, the channel walls 238 extend perpendicular to the circular walls 233 in the direction of the ramp portion 216, and the outflow channel 231 is positioned through a gap in the circular walls 233. In some embodiments, the channel walls 238 align (e.g., abut) with the ramp walls 217 to further direct bodily fluid flow. In other embodiments, the ramp walls 217 are spaced farther apart than the channel walls 238, such as in FIG. 7B. With reference to FIG. 2, the circular walls 233 are also configured to direct fluid out of the floating island 230 towards the collection reservoir 106. In some embodiments, the outflow channel 231 can be at an angle relative to the skin 101 (e.g., not parallel) to align with the ramp portion 216 and/or to increase the smooth (e.g., laminar) flow of fluid in the device 100.

In the illustrated embodiment, between the outflow channel 231 and the ramp portion 216 is an exposed portion 223 (e.g., exposed to blood flowing through the housing 102) of the membrane 212 on the outside of the first tier 221. In the illustrated embodiment, the channel walls 238 include flanges 239 that extend beyond the footprint of the floating island 230 over the exposed portion 223 of the membrane 212. In some aspects of the present technology, the flanges 239 can direct fluid toward the ramp portion 216 and the outflow port 215 when there is a large stream of fluid from the wound. For example, when the stream is high enough to contact the flanges. This inhibits large bodily fluid flows from immediately spreading beyond a footprint of the ramp portion 216 once the fluid has passed the channel walls 238. This can also inhibit blood from collecting adjacent to the outflow port 215 and/or degrading from prolonged contact with surfaces of the housing 102. As shown in FIG. 7B, in some embodiments, the flanges 239 contact the exposed portion 223 when the membrane 212 is drawn upwards due to vacuum within the housing 102. In some aspects of the present technology, the flanges 239 aid in directing fluid to flow towards the ramp portion 216 and inhibit fluid from spreading on the membrane 212 and/or flowing to the sides of the outflow port 215. In other embodiments, the flanges 239 are omitted.

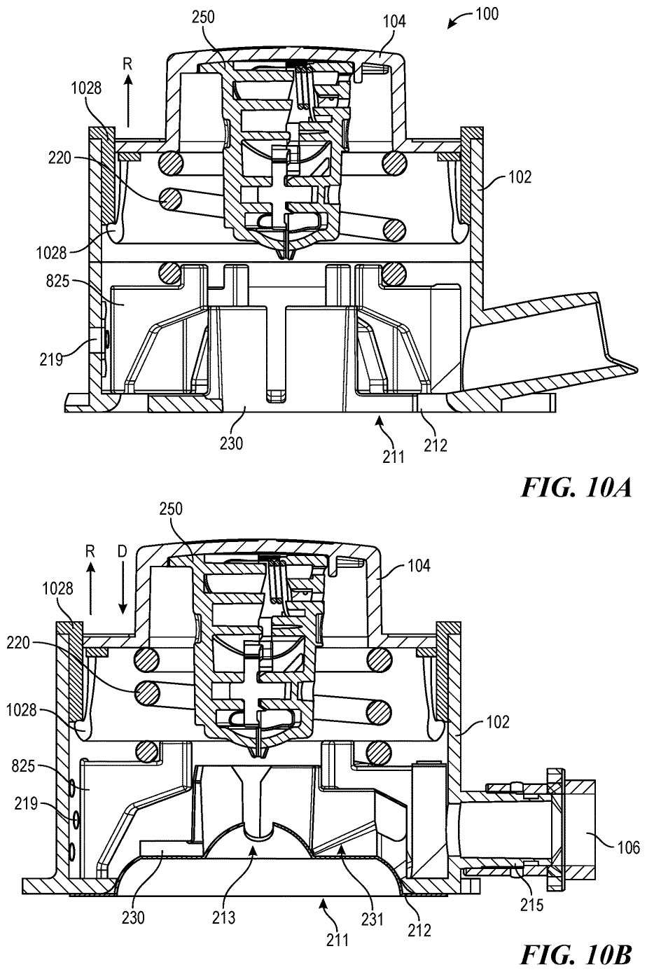

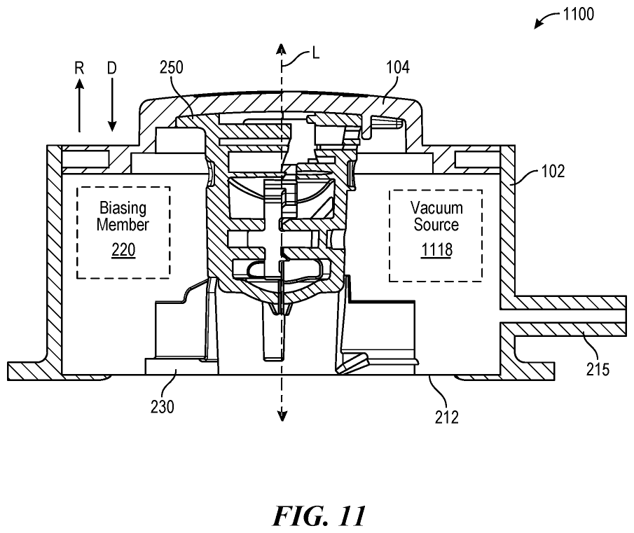

With reference to FIG. 2, the housing 102 houses a vacuum source 218 that includes a vent 219 (e.g., an umbrella valve) configured to allow one-directional fluid flow (e.g., allow outflow from the housing 102 but inhibit inflow into the housing 102). In the illustrated embodiment, the vacuum source 218 includes a biasing member 220 (e.g., a spring) configured to bias the actuator 104 in the retraction direction R. The housing 102 is also sealingly coupled to the actuator 104 with a flexible seal 1028 (FIGS. 10A and 10B) that can flex as the actuator 104 moves in the deployment direction D and the retraction direction R without the seal breaking between the housing 102 and the actuator 104. As described in greater detail below with reference to FIGS. 10A and 10B, the vacuum source 218 generates vacuum in the housing 102 and the actuator 104 by pushing air out of the housing 102 through the vent 219 when the actuator 104 is depressed (e.g., moved in the deployment direction D) and generating negative pressure when the actuator 104 is biased in the retraction direction R by the biasing member 220 while the vent 219 inhibits air flow into the housing 102 and/or the actuator 104. In some embodiments, portions of the actuator 104 can be fluidly disconnected from portions of the housing 102. For example, movement in the deployment direction D could expel air only from the housing 102 while the ambient air in the actuator 104 remains unaffected. In some aspects of the present technology, this can be used to control the amount of air that is forced out of the vent 219 and, consequently, the degree of negative pressure that is generated. In other embodiments, the vacuum source 218 can include different components that are configured to generate a vacuum when the actuator 104 is actuated (e.g., depressed, rotated, pressed), such as systems that apply preexisting negative pressure to components of the housing 102, like the vacuum source 1118 of FIG. 11.

FIG. 3 is a schematic cross-sectional top view of the bodily fluid collection device 100 of FIG. 1A configured in accordance with embodiments of the present technology. In the illustrated embodiment, the floating island 230 has an island inner diameter D_{II} and an island outer diameter D_{IO}. The island inner diameter D_{II} represents the distance between opposite (e.g., opposing) surfaces of the circular walls, and the island outer diameter D_{IO} represents the diameter of the outer perimeter (e.g., edge) of the flat portion 232. In the illustrated embodiment, the island inner diameter D_{II} is 11.6 mm and the island outer diameter D_{IO} is 25 mm. In other embodiments, the island inner diameter D_{II} can be varied to reduce bruising for the patient. For example, a wider island inner diameter D_{II}, for example, 12 mm or 13 mm, will create a larger (e.g., wider, greater area) cutting region 235 and a larger (e.g., wider, greater surface area) second tier 222, decreasing the amount of vacuum (e.g., negative pressure) applied to the skin 101 per square inch. The island outer diameter D_{IO} can be varied to accommodate larger and smaller island inner diameters D_{II}. In some aspects of the present technology, increasing the island inner diameter D_{II} can decrease the amount/risk of bruising of the skin 101.

The device 100 further has a vacuum diameter D_{V} that represents the diameter of the opening 211 (e.g., the area on which vacuum is applied to the skin 101). Generally, the vacuum diameter D_{V} is proportional to the amount of blood that is collected during the operation of the device 100. In the illustrated embodiment, the vacuum diameter D_{V} is 30 mm. In other embodiments, the vacuum diameter D_{V} can be 20 mm, 25 mm, 35 mm, 40 mm, or any diameter therebetween. Increasing the vacuum diameter D_{V}, however, generally increases the size of the device 100 and can be detrimental in some applications. For example, in applications where blood collection is intended to be done on surfaces without large, flat sections (e.g., fingers) and/or when used by patients with limited mobility. The island outer diameter D_{IO} can be approximately 70% (seventy percent) of the vacuum diameter D_{V}. In other embodiments, the island outer diameter D_{IO} can be 65% of the vacuum diameter D_{V}, 75% of the vacuum diameter D_{V}, or any percentage therebetween. The difference between the vacuum diameter D_{V} and the island outer diameter D_{IO} represents a planar width of the exposed portion 223 (FIG. 2). In some aspects of the present technology, reducing the planar width of the exposed portion 223 can decrease the degradation of collected blood caused by exposure to the tacky material on the inner surface of the membrane 212 (FIG. 2). More specifically, decreasing the difference between the vacuum diameter D_{V} and the island outer diameter D_{IO} can reduce the degradation of the collected sample. However, decreasing this difference may also reduce the amount of blood collected.

In the illustrated embodiment, the device 100 has a base inner diameter D_{BI} that is 40.12 mm. The base inner diameter D_{BI} represents the distance between opposing inner walls of the housing 102. The smaller the base inner diameter D_{BI}, the smaller the device 100, however, the base inner diameter D_{BI} is generally the diameter necessary to accommodate the dimensions of components of the housing 102 (e.g., the ramp portion 216) and/or the device 100 (e.g., the skin-piercing assembly 250). In some embodiments, the base inner diameter D_{BI} is the same as the vacuum diameter D_{V}, and the outflow channel 231, the ramp portion 216, and/or the flanges 239 are excluded.

In the illustrated embodiment, the device 100 has a sealing width W_{S} that is 10 mm. The sealing width W_{S} represents the width of a ring coaxial with the axis L (FIG. 2). The bottom surface of the housing 102 within the ring adheres to the skin 101 and vacuum is not applied to the skin 101 within the ring. The area of this ring is thus proportional to the effectiveness of the adhesion of the device 100 to the skin 101. The minimum size of the sealing width W_{S} is equal to an outer diameter D_{H} of the housing 102 minus the vacuum diameter D_{V}. In some embodiments, the device 100 can have a sealing width W_{S} that is the minimum size, or it can have a sealing width W_{S} greater than the minimize size.

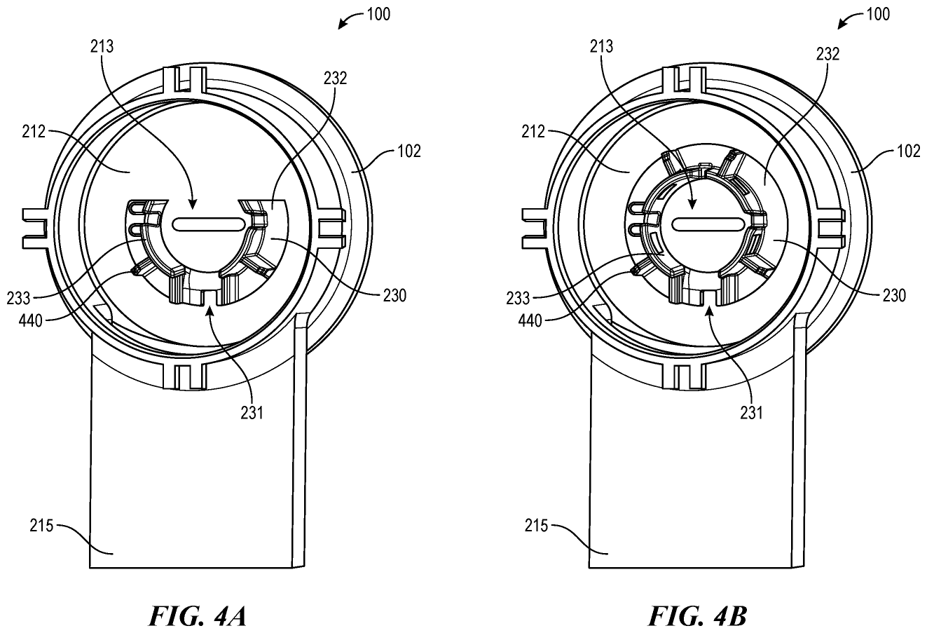

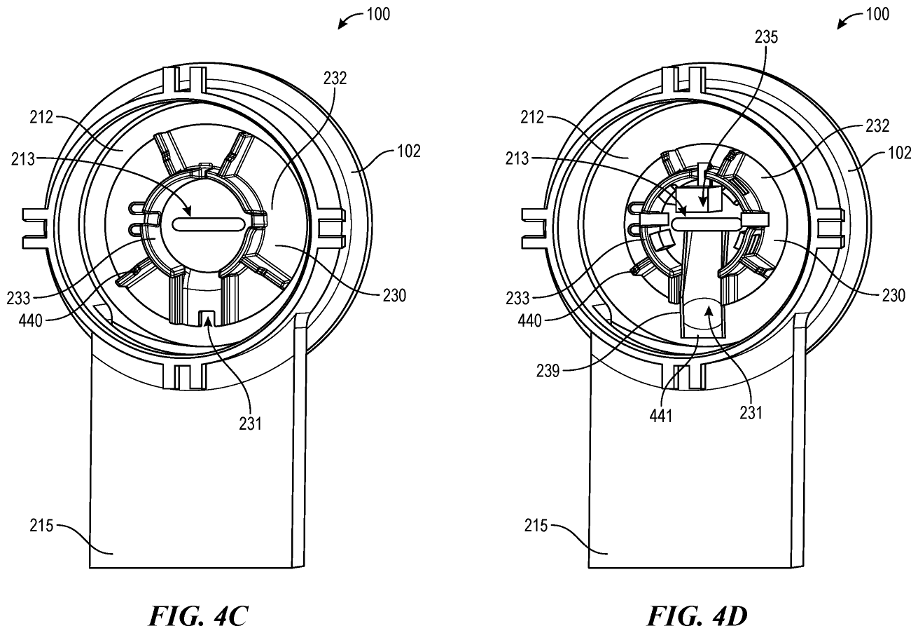

FIGS. 4A-4D are schematic perspective top views of floating islands 230 of the bodily fluid collection device 100 of FIG. 1A configured in accordance with embodiments of the present technology. With reference to FIGS. 4A-4D, the outflow ports 215 are shown as simplified rectangular flanges, however, it should be understood that the outflow ports 215 can include features similar or identical to the outflow port 215 of FIG. 2. Further, the housings 102 are shown with base inner diameters D_{BI} (FIG. 3) equal to the vacuum diameters D_{V} (FIG. 3), however, it should be understood that the floating islands 230 of FIGS. 4A-4D could be incorporated into housings 102 with base inner diameters D_{BI} less than or greater than the vacuum diameters D_{V}. In the illustrated embodiments, the floating islands 230 have vacuum diameters D_{V} equal to 30 mm. The floating islands 230 are coaxial with the membranes 212 (e.g., sharing a common center) and are centered around the precut hole 213. The outflow channels 231 also align with (e.g., lead to) the outflow ports 215. The circular walls 233 can include protruding support structures 440. The support structures 440 can be configured to inhibit the flat portion 232 of the floating islands 230 from bending when subject to vacuum. In some embodiments, the support structures 440 can be omitted.

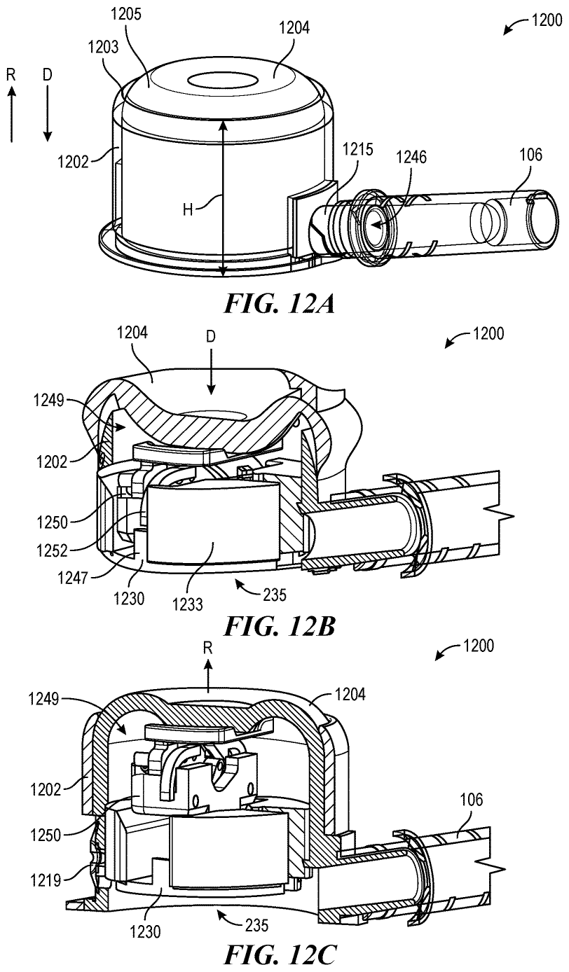

With reference to FIG. 4A, the floating island 230 is hemispherical (e.g., semi-circular; half-moon shaped), as such the top half (e.g., the half opposite the outflow port 215) is omitted. In some aspects of the present technology, this can allow different configurations of the skin-piercing assembly 250 (FIG. 2) to be used as there are fewer potential obstructions between the skin-piercing assembly 250 and the skin 101 (FIG. 1B) (e.g., portions of the floating island 230). In the illustrated embodiment, the floating island 230 creates a modified tiered shape of the membrane 212 when vacuum is present in the housing 102. For example, on the bottom half (e.g., the half with floating island 230), the membrane 212 will have the two-tiered shape described in detail above with reference to FIG. 2. On the top half (e.g., the half without the floating island 230), the membrane 212 will be a single-tier shape (e.g., a continuous curved shape). In some aspects of the present technology, this configuration can reduce trauma to the patient's skin 101 by allowing the vacuum pressure applied to the top half of the skin 101 to be applied in a uniform manner while maintaining benefits in blood flow generated by the floating island 230, as described in greater detail below with reference to FIGS. 10A, 10B, and 12C. In other embodiments, the half-moon shaped floating island 230 can be rotated about the axis L (FIG. 2) to other orientations on the membrane 212. For example, the floating island 230 can be on the top half of the membrane 212. In such embodiments, the outflow channel 231 can be moved to maintain alignment with the outflow port 215 or omitted.

With reference to FIG. 4B, the floating island 230 is circular. More specifically, FIG. 4B is a complete circular version of the floating island 230 of FIG. 4A. In some embodiments, the floating island 230 of FIG. 4B can have features similar or identical to the floating island 230 of FIG. 2, and/or the floating island 230 of FIG. 4A.

With reference to FIG. 4C, the floating island 230 has an island outer diameter D_{IO} (FIG. 3) 5 mm greater than the island outer diameter D_{IO} (FIG. 3) of the floating islands 230 of FIGS. 4A, 4B, and 4D. For example, the island outer diameter D_{IO} of the floating island 230 in FIG. 4C is 30 mm, while the island outer diameter D_{IO} of the floating islands 230 of FIGS. 4A, 4B, and 4D are 25 mm. In other embodiments, the island outer diameter D_{IO} of the floating island 230 of any of FIGS. 4A-4D can be 20 mm, 21 mm, 22 mm, 23 mm, 24 mm, 26 mm, 27 mm, 28 mm, 29 mm, 31 mm, 32 mm, 33 mm, 34 mm, 35 mm, or any measurement therebetween. With reference to FIG. 4C, the increase in the island outer diameter D_{IO} is created by increasing the width (e.g., the radial width) of the flat portion 232. In other embodiments, the island outer diameter D_{IO} can be increased by increasing the thickness of the circular walls 233 and/or the support structures 440. Generally, increased island outer diameters D_{IO} (e.g., the size of the floating island 230) are correlated with increased blood flow rates. Thus, in some aspects of the present technology, increasing the island outer diameter D_{IO} increases bodily fluid flow rates, as described below with reference to FIG. 12C.

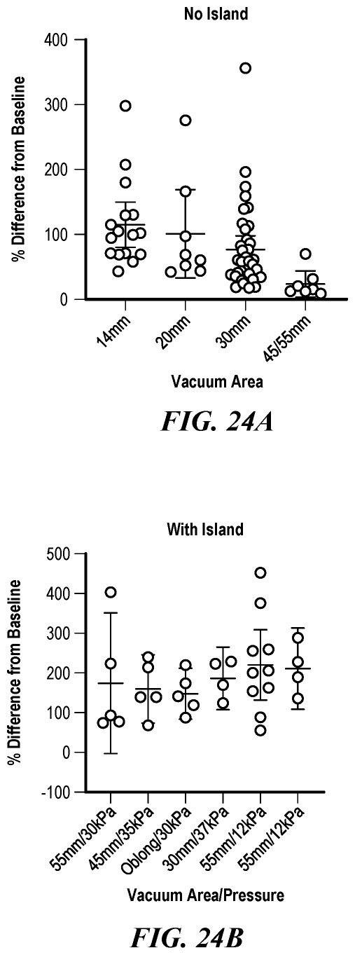

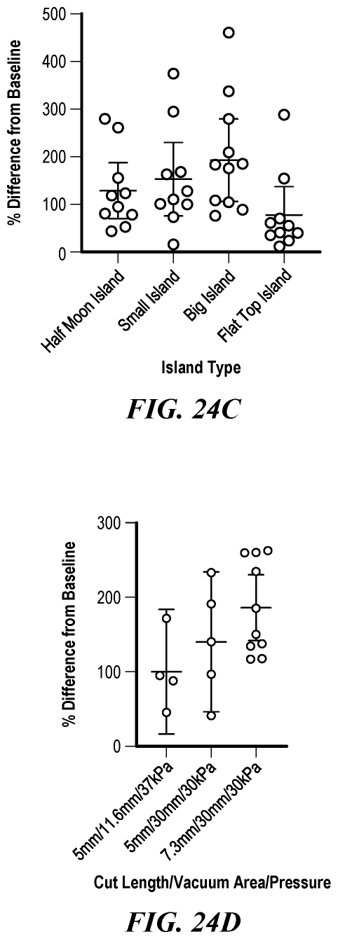

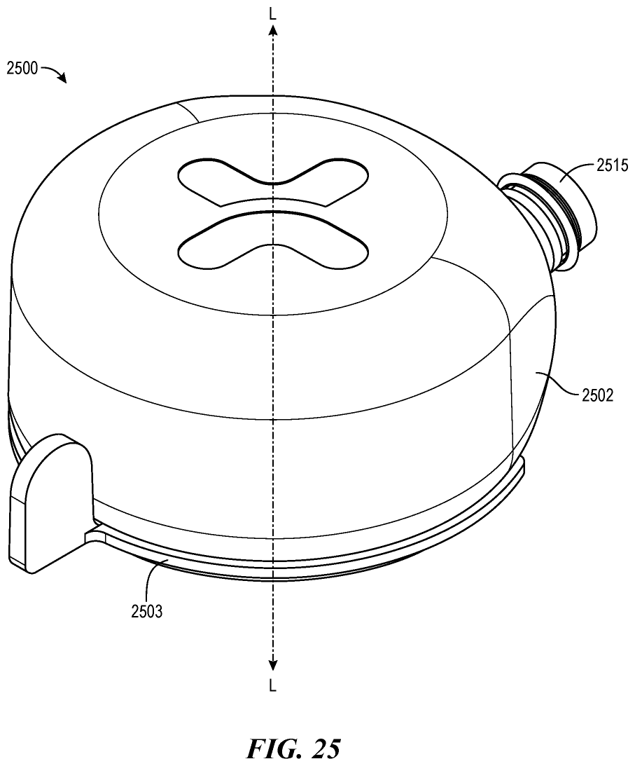

With reference to FIG. 4D, the island inner diameter D_{II} (FIG. 3) is reduced to 0 mm. As such, the inner edge of the floating island 230 abuts the precut hole 213. In other embodiments, the precut hole 213 can be a different shape, size, and/or number (e.g., two or more precut holes 213), and the inner edge of the floating island 230 can be adjusted to abut the outer edge(s) of the precut hole(s) 213. In the illustrated embodiment, the flat portion 232 extends into the cutting region 235 to reduce the island inner diameter D_{II}. In other embodiments, the circular walls 233 and/or support structures 440 can extend into the cutting region 235 to reduce the island inner diameter D_{II}. Generally, reduced island inner diameters D_{II} (e.g., the size of the floating island 230) are correlated with increased blood flow rates. Thus, in some aspects of the present technology, decreasing the island inner diameter D_{II} increases bodily fluid flow rates, as described below with reference to FIG. 24C.

With reference to FIG. 4D, the floating island 230 further includes the flanges 239 and an extending portion 441 of the flat portion 232. The extending portion 441 is configured to bridge the gap between the outflow channel 231 and the ramp portion 216 (FIG. 2). More specifically, the extending portion 441 creates a walled-in channel between the outflow channel 231 and the ramp portion 216 so that fluid can flow between the outflow channel 231 and the ramp portion 216 without contacting the membrane 212. In some embodiments, the extending portion 441 includes a curved section adjacent to the membrane 212 to accommodate the domed shape of the exposed portion 223 (FIG. 2). In other embodiments, the extending portion 441 includes the film bridge 743 of FIG. 7C.

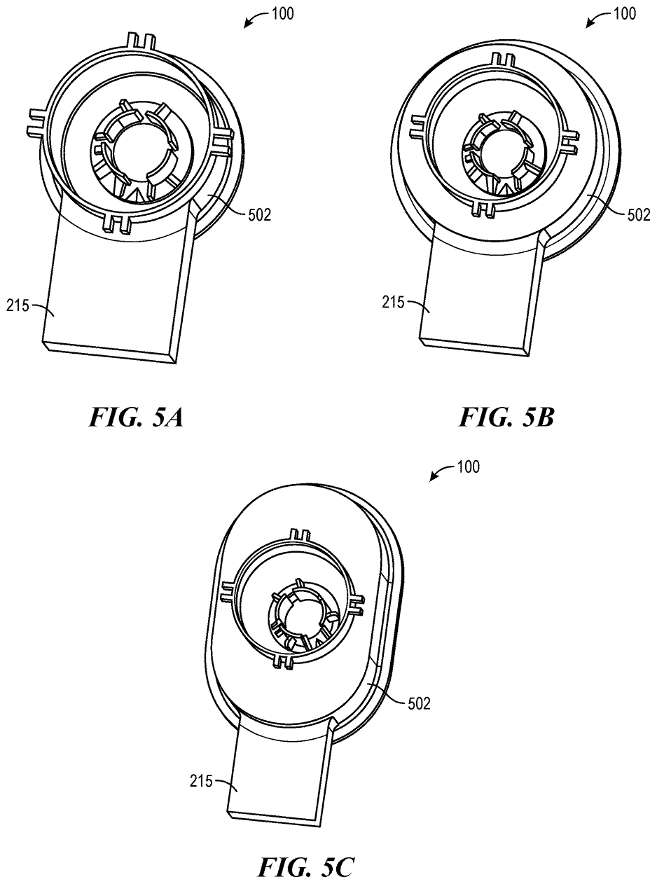

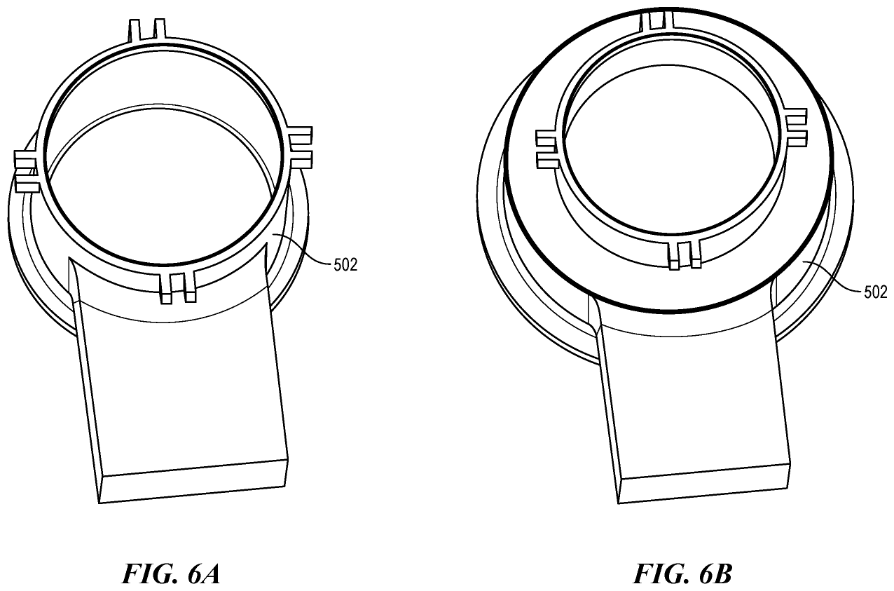

FIGS. 5A-5C are schematic perspective top views of housings 502 of the bodily fluid collection device 100 of FIG. 1A configured in accordance with embodiments of the present technology. With reference to FIGS. 5A-5C, the outflow ports 215 are shown as simplified rectangular flanges, however, it should be understood that the outflow ports 215 can include features similar or identical to the outflow port 215 of FIG. 2. In the illustrated embodiments, the housings 502 of the devices 100 have vacuum diameters D_{V} (FIG. 3) of varying sizes, as described in greater detail below. The housings 502 can include some features that are at least generally similar in structure and function, or identical in structure and function, to the corresponding features of the housing 102 described in detail above with reference to FIGS. 1A-4D and 6D-11 and can operate in a generally similar or identical manner to the housing 102.

With reference to FIG. 5A, the housing 502 is circular and has a uniform vacuum diameter of 30 mm. With reference to FIG. 5B, the housing 502 is circular and has a uniform vacuum diameter D_{V} of 45 mm. With reference to FIG. 5C, the housing 502 is oblong (e.g., ovular) and has a varying vacuum diameter D_{V}. The vacuum diameter D_{V} of the narrower section of the oblong shape is 42 mm. Generally, increasing the vacuum diameter D_{V} and/or vacuum area can increase bodily fluid flow rates when the device 100 has a floating island 230. Thus, in some aspects of the present technology, increasing the vacuum diameter D_{V} can increase bodily fluid flow rates, as described below with reference to FIG. 24B.

With reference to FIGS. 5B and 5C, the housings 502 are wider at the bottom (e.g., the portion of the housings 102 contacting the skin 101 (FIG. 1B)) than at the top (e.g., the portion adjacent to the actuator 104 (FIG. 2)). In some aspects of the present technology, this increases the size of the vacuum diameter D_{V} without increasing a diameter of the entire device (e.g., the outer housing diameter D_{H} (FIG. 3)) to be equal to the vacuum diameter D_{V}. Thus, this configuration can maintain a desirable form factor of the device 100 while increasing bodily fluid flows. In other embodiments, the housings 502 of FIGS. 5A-5C can be used in the device 100 of FIG. 2, and/or features of the housings 502 of FIGS. 5A-5C can be incorporated into the housing 102 of FIG. 2.

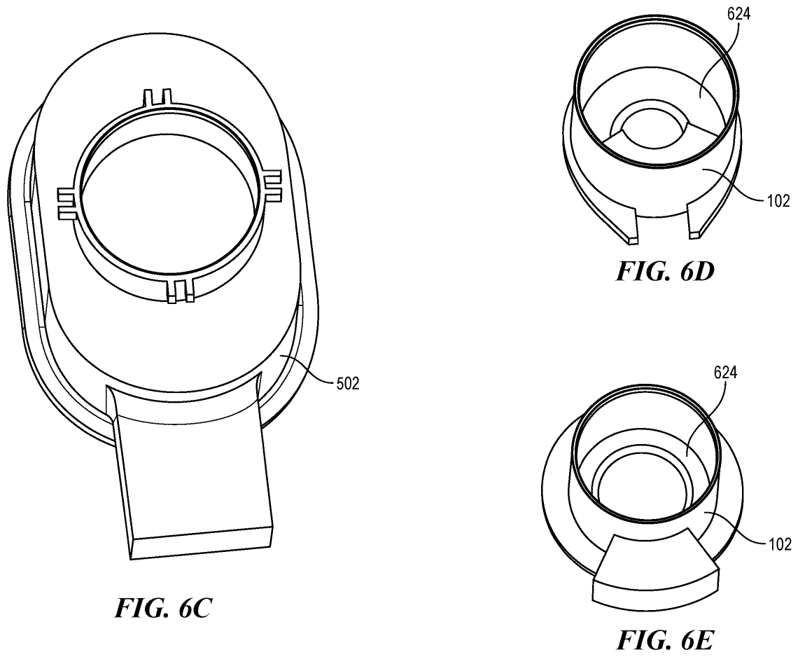

FIGS. 6A-6C are schematic perspective top views of the housings 502 of FIGS. 5A-5C , respectively, without the floating islands 230 configured in accordance with embodiments of the present technology. With reference to FIGS. 6A-6C, the outflow ports 215 are shown as simplified rectangular flanges, however, it should be understood that the outflow ports 215 can include features similar or identical to the outflow port 215 of FIG. 2. In the illustrated embodiments, the vacuum diameters D_{V} (FIG. 3) of the housings 502 of FIGS. 6A-6C are the same as the vacuum diameters D_{V} of the housings 502 of FIGS. 5A-5C, respectively. As described in detail below with reference to FIGS. 24A and 24B, the absence of the floating islands 230 can decrease the bodily fluid flow rates of the device 100.

FIGS. 6D and 6E are schematic perspective top views of the housings 102 of the bodily fluid collection device 100 of FIG. 1A configured in accordance with embodiments of the present technology. With reference to FIGS. 6D and 6E, the housings 102 have a base inner diameter D_{BI} (FIG. 3) greater than the vacuum diameter D_{V} (FIG. 3). Further, the housings 102 have inner flat portions 624 that extends towards the center of housing 102 from the bottom of the housing 102. The inner flat portions 624 effectively reduces the vacuum diameters D_{V}. With reference to FIG. 6D, the vacuum diameter D_{V} is 20 mm. With reference to FIG. 6E, the vacuum diameter D_{V} is 14 mm. As described below with reference to FIG. 12A, decreasing the vacuum diameter D_{V} can increase the bodily fluid flow rates of the device 100 when the device 100 does not have the floating island 230.

FIGS. 7A-7C are schematic perspective top views of the floating islands 230 of the bodily fluid collection device 100 of FIG. 1A configured in accordance with embodiments of the present technology. In the illustrated embodiments, vacuum is being applied to the membrane 212 and the floating island 230 such that the membrane 212 has the two-tiered shape described in detail above with reference to FIG. 2. With reference to FIGS. 7A and 7B, the housing includes the ramp walls 217. With reference to FIGS. 7B and 7C, the floating islands 230 include the channel walls 238.

With reference to FIG. 7A, the channel walls 238 and the flanges 239 are omitted. Further, the flat portion 232 of the floating island 230 defines a cutout portion 742 where the flat portion 232 is omitted. Thus, when blood flows from the precut hole 213 towards the outflow port 215 at the bottom of the device 100, the blood flows off the second tier 222 of the membrane 212, through the cutout portion 742 to the first tier 221 of the membrane 212, and onto the ramp portion 216 of the housing 102 between the ramp walls 217 to the outflow port 215. In some aspects of the present technology, the omission of channel walls 238 creates a laminar flow of bodily fluid into the outflow port 215.

With reference to FIG. 7B, the floating island 230 includes the flanges 239 and the cutout portion 742 of the flat portion 232 is omitted (e.g., the flat portion 232 extends around the entire circumference of the floating island 230). Thus, when blood flows from the precut hole 213 towards the outflow port 215 at the bottom of the device 100, the blood flows from the second tier 222 of the membrane 212 to the outflow channel 231, the first tier 221 of the membrane 212, onto the ramp portion 216 of the housing 102 between the ramp walls 217, and to the outflow port 215. In some aspects of the present technology, the channel walls 238 and the flanges 239 create guides for the fluid to flow into the outflow port 215.

With reference to FIG. 7C, the device 100 includes a film bridge 743 between the outflow channel 231 of the floating island 230 and the ramp portion 216 of the housing 102. The film bridge 743 can have properties (e.g., textures, chemical compositions) that protect collected blood from degradation (e.g., inhibit degradation). Further, the film bridge 743 can be configured to utilize the surface tension of the flowing blood to reduce the amount of blood that travels off the film bridge 743 and onto other components of the device 100 (e.g., the membrane 212). For example, the film bridge 743 can be separated from the surface of the floating island 230 and/or the housing 102 to create a distinct edge along the sides of the film bridge 743. As such, the surface tension of the blood will, to an extent, keep the blood flowing along the top surface of the film bridge 743. In the illustrated embodiment, when blood flows from the precut hole 213 towards the outflow port 215 at the bottom of the device 100, the blood flows from the second tier 222 of the membrane 212 to the outflow channel 231 and along the film bridge 743 to the outflow port 215. In some embodiments, the film bridge 743 can include one or more features that are generally similar to or identical to the coating or membrane disclosed in International Patent Application No. PCT/US24/47665, titled “BODILY FLUID COLLECTION DEVICES FOR COLLECTING HIGH QUALITY BLOOD FOR COMPLETE BLOOD COUNT (CBC) TESTING, AND RELATED METHODS,” and filed Sep. 20, 2024, which is incorporated herein by reference in its entirety.

FIG. 7D is a perspective top view of the floating island 230 of the bodily fluid collection device 100 of FIG. 1A configured in accordance with embodiments of the present technology. In the illustrated embodiment, the channel walls 238 (FIG. 7B) are omitted and the flat portion 232 defines the cutout portion 742, similar to the floating island 230 of FIG. 7A, however, it should be understood that the channel walls 238 and/or other features of the floating islands 230 of FIGS. 2-4D and 7A-7C could be included.

In the illustrated embodiment, the floating island 230 includes a stepped portion 744. The stepped portion 744 extends circumferentially around the floating island 230 and has a thickness generally less than the thickness of the flat portion 232. In some embodiments, the stepped portion 744, the flat portion 232, and/or a bottom surface of the floating island 230 can be overmolded with a flexible elastomer (e.g., TPU, Santoprene). In some aspects of the present technology, the flexible elastomer can reduce bruising when vacuum pressure is applied to the skin 101 (FIG. 2) by creating a softer (e.g., compressible) contact surface between the floating island 230 and the skin 101. In other embodiments, the floating island 230 can have the flexible elastomer adhered to the stepped portion 744, the flat portion 232, and/or the bottom surface of the floating island 230 via adhesive (e.g., glue) or other molding techniques. In some embodiments, the edges (e.g., corners, outer circumference) of the stepped portion 744 can be rounded or chamfered to further reduce the risk of bruising.

FIGS. 8A and 8B are schematic cross-sectional side views of the bodily fluid collection device 100 of FIG. 1A configured in accordance with embodiments of the present technology. With reference to FIGS. 8A and 8B, the outflow port 215 is coupled to the housing 102 at a 10-degree angle. As shown in FIG. 8B, coupling the outflow port 215 to the housing 102 at an angle reduces the difference in angle between the ramp portion 216 and the outflow port 215. More specifically, with reference to FIG. 8B, the transition from the ramp portion 216 to the outflow port 215 creates a lip 845. When the outflow port 215 is parallel to the bottom of the housing 102, the lip 845 can affect bodily fluid flow between the ramp portion 216 and the outflow port 215. For example, if the lip 845 is significant, the lip 845 can disrupt the laminar flow of bodily fluid from the floating island 230 to the outflow port 215. In some aspects of the present technology, coupling the outflow port 215 at an angle reduces the lip 845 to improve bodily fluid flow from the ramp portion 216 to the outflow port 215, inhibiting build-up on the ramp portion 216 and/or overflow of the ramp portion 216 that could lead to degradation of the sample.

With reference to FIGS. 8A and 8B, the housing 102 also includes one or more retention arms 825. Each of the retention arms 825 includes a protruding portion 826 at the end of the retention arm 825. In the illustrated embodiment, there are eight retention arms 825 (four shown and four hidden). The retention arms 825 are configured to hold the biasing member 220 (e.g., a spring; FIGS. 10A and 10B) above and away from the floating island 230 when vacuum is not being applied to the membrane 212 (FIG. 2). The retention arms 825 also act as a stop for the biasing member 220 in the deployment direction D so that the biasing member 220 can become compressed (e.g., actuated, activated, generating biasing force). Further, in the illustrated embodiment, the protruding portion 826 inhibits the biasing member 220 from sliding within the housing by contacting inner surfaces of the biasing member 220. When vacuum is applied to the membrane 212, the circular walls 233 (FIG. 8A) of the floating island 230 can rise adjacent to the protruding portion 826. In some embodiments, the retention arms 825 and/or protruding portions 826 can act as guides to ensure that the floating island 230 moves in the retraction direction R perpendicular to the skin 101 (FIG. 1A). In some aspects of the present technology, the retention arms 825 inhibit the floating island 230 from becoming askew (e.g., unaligned with the axis L), which could inhibit blood flow towards the outflow port 215. For example, if the floating island 230 tilts away from the outflow port 215, blood may collect within the floating island 230 and/or be positioned out of the floating island 230 towards the vent 219 (FIG. 8A).

FIGS. 9A-9J are schematic top views of cuts 951 created by the bodily fluid collection device 100 of FIG. 1A configured in accordance with embodiments of the present technology. The effects of varying cuts 951 are described below with reference to FIG. 24D. With reference to FIGS. 9A-9J, the outflow ports 215 are shown as simplified rectangular flanges, however, it should be understood that the outflow ports 215 can include features similar or identical to the outflow port 215 of FIG. 2. Further, the housings 102 are shown with base inner diameters D_{BI} equal to the vacuum diameters D_{V}, however, it should be understood that the cuts 951 of FIGS. 9A-9J could be incorporated into housings 102 with base inner diameters D_{BI} less than or greater than the vacuum diameters D_{V}. For example, the cuts 951 of FIG. 9F could be made by the device 100 of FIG. 2 by making the cuts 951 adjacent to the circular walls 233 (FIG. 2) of the floating island 230 (FIG. 2). In other embodiments, such as the housing 502 of FIG. 6A, the cuts 951 of FIG. 9F could be made by making the cuts 951 adjacent to the bottom interior edge of the housing 502 (FIG. 6A).

With reference to FIGS. 9A-9J, the illustrated embodiments include the housings 102 and the membranes 212. The skin 101 (FIG. 1A) also includes the cuts 951. The cuts 951 can be created by one or more blades of the skin-piercing assembly 250 (FIG. 2) and can be configured in a variety of orientations depending on factors such as the patient's age, health, risk factors; the area of application; user preference; and/or the like. With reference to FIGS. 9A-9J, the cuts 951 are of uniform shape, depth, and length. In the illustrated embodiments, the cuts 951 are straight lines that are 0.2 mm deep and 1 mm long. In other embodiments, the cuts 951 can be curved, chevron-shaped, and/or the like. In some embodiments, the cuts 951 can have a depth of 0.1 mm, 0.3 mm, 0.4 mm, 0.5 mm, 0.6 mm, or any depth therebetween. In further embodiments, the cuts 951 can have a length of 0.3 mm, 0.4 mm, 0.5 mm, 0.6 mm, 0.7 mm, 0.8 mm, 0.9 mm, 1.1 mm, 1.2 mm, 1.3 mm, 1.4 mm, 1.5 mm, or any length therebetween. In some embodiments, the cuts 951 are combinations of the above.

With reference to FIG. 9A, the cut 951 is in the center of the membrane 212 and the cut 951 is parallel to an outlet 927 of the outflow port 215. With reference to FIG. 9B, there are two cuts 951 side by side with a small gap dividing them. The cuts 951 are also parallel to the outlet 927. With reference to FIG. 9C, the cut 951 is at the bottom of the membrane 212, close to the outflow port 215 and parallel to the outlet 927. In the illustrated embodiment, the cut 951 is 1 mm from the outflow port 215. In other embodiments, the cut 951 of FIG. 9C can have a distance from the outflow port 215 of 2 mm, 3 mm, 4 mm, 5 mm, 6 mm, 7 mm, 8 mm, 9 mm, 10 mm, or any distance therebetween.

With reference to FIG. 9D-9F, there are two stacked cuts 951, both positioned in parallel to the outlet 927. The cuts 951 of FIG. 9E are placed closer together than the cuts 951 of FIG. 9D. For example, the cuts 951 of FIG. 9E could be 2 mm apart while the cuts 951 of FIG. 9D could be 4 mm apart. The cuts 951 of FIG. 9F are placed farther apart than the cuts 951 of FIG. 9D. For example, the cuts 951 of FIG. 9F could be 6 mm apart while the cuts 951 of FIG. 9D could be 4 mm apart. In other embodiments, the cuts 951 of FIGS. 9D-9F could be placed apart by 1 mm, 3 mm, 5 mm, 7 mm, 9 mm, or any distance therebetween.

With reference to FIG. 9G, the cut 951 in the center of the membrane 212 and the cut 951 is perpendicular with the outlet 927. With reference to FIG. 9H, the cut 951 is in the center of the membrane 212 and the cut 951 is at a 45-degree angle relative to the outlet 927. In other embodiments, the cut 951 can be rotated to be at any angle between 0 and 360 degrees relative to the outlet 927. With reference to FIG. 9I, there are two cuts 951 offset from each other and at 45-degree angles relative to the outlet 927. In other embodiments, the cuts 951 of FIG. 9I can be offset to the same degree as the cuts 951 of FIGS. 9D-9F. With reference to FIG. 9J, one of the cuts 951 is parallel to the outlet 927 and offset from the center of the membrane 212 by 3 mm. There is also a cut 951 that is at a 45-degree angle relative to the outlet 927 and offset from the center of the membrane 212 by 3 mm. In other embodiments, the cuts 951 of FIG. 9J can be horizontally offset from the center of the membrane 212 by 1 mm, 2 mm, 4 mm, 5 mm, 6 mm, 7 mm, 8 mm, 9 mm, 10 mm, 15 mm, or any distance therebetween. In other embodiments, the cuts 951 of FIG. 9J can be combined with and/or replace one or more cuts 951 of FIGS. 9A-9I.

FIGS. 10A and 10B are cross-sectional side views of the housing 102 of FIG. 2 in a first position and a second position, respectively, configured in accordance with embodiments of the present technology. With reference to FIGS. 10A and 10B, the housing 102 includes the flexible seal 1028, described above with reference to FIG. 2. The flexible seal 1028 extends circumferentially around the housing 102. In other embodiments, the flexible seal 1028 can be an O-ring coupled circumferentially around the actuator 104 to sealingly couple the actuator 104 to the housing 102. In the illustrated embodiment, the housing 102 also includes the retention arms 825, described in detail above with reference to FIGS. 8A and 8B. In the illustrated embodiments, the device 100 and one or more of its components (e.g., the housing 102 (FIG. 1A), the housing 102, the membrane 212) rests on the patient's skin 101 (FIG. 2) and is sufficiently adhered to the skin 101 such that the device 100 remains in place as the patient moves and/or releases the device 100, even when the device 100 is placed on vertical surfaces (e.g., the resting arm of a standing patient).

With reference to the FIG. 10A, the skin-piercing assembly 250 is undeployed (e.g., not actuated, activated) and hovers over the floating island 230. The biasing member 220 rests on the retention arms 825 in an uncompressed position and provides a minor biasing force against the actuator 104 in the retraction direction R. In some embodiments, the biasing member 220 can be completely uncompressed in the first position. In the illustrated embodiment, the membrane 212 and the floating island 230 are flat against the patient's skin 101 and the housing 102 is at atmospheric pressure (e.g., no vacuum).

With reference to FIG. 10B, the actuator 104 has been fully pressed (e.g., depressed, actuated) in the deployment direction D and released. Upon release, the biasing member pushes the actuator 104 in the retraction direction R, returning the actuator 104 to the same location as in the first position. More specifically, in the second position, the actuator 104 is in the same position as in the first position. When the actuator 104 is fully pressed in the deployment direction D, the skin-piercing assembly 250 deploys (e.g., activates, releases one or more blades) through the precut hole 213 in the membrane 212 and the skin 101 is cut. Thus, in the second position, the resulting wound is bleeding, and blood is flowing into the housing 102.

During the operation of device 100, the actuator 104 is depressed in the deployment direction D from the first position and fluid (e.g., ambient air) is forced out of the one-way vent 219. When the actuator 104 is released and biased in the retraction direction R by the biasing member 220 into the second position, vacuum is generated in the housing 102 and/or the actuator 104. The negative pressure of the vacuum draws the membrane 212, the floating island 230, and the skin 101 upward into the housing 102 through the opening 211. This suction is facilitated by the adhesives on the bottom of the membrane 212 and the housing 102. As described in detail above with reference to FIG. 2, the vacuum within the housing 102 creates the two-tiered shape of the membrane 212. The patient's skin 101 is similarly drawn upward in the same two-tiered shape because the membrane 212 is adhered to the skin 101 across its entire bottom surface. Further, since the wound is bleeding, blood will flow from the floating island 230 through the outflow channel 231 to the outflow port 215 and into the collection reservoir 106. When the blood collection has finished, the patient can remove the device 100 from their skin by lifting one end and slowly breaking the adhesive connection until the entire device 100 is unstuck from their skin 101. In some aspects of the present technology, the two-tiered shape of the membrane 212 and the skin 101 increases the bodily fluid flow rates of the device 100 when compared to devices without floating islands 230 (e.g., housings 502 of FIGS. 5A-5C), as described below with reference to FIGS. 24A and 24B.

FIG. 11 is a schematic cross-sectional side view of a bodily fluid collection device 1100 configured in accordance with embodiments of the present technology. The device 1100 can include some features that are at least generally similar in structure and function, or identical in structure and function, to the corresponding features of the device 100 described in detail above with reference to FIGS. 1A-10B, and can operate in a generally similar or identical manner to the device 100. For example, in the illustrated embodiment, the device 1100 includes the housing 102, the floating island 230 adhesively coupled to the membrane 212, the skin-piercing assembly 250, and the outflow port 215.

In the illustrated embodiment, the skin-piercing assembly 250 has not been actuated and the skin has not been cut, similar to the first position described in detail above with reference to FIG. 10A. In the illustrated embodiment, a vacuum source 1118 includes the biasing member 220 but the vents 219 of FIGS. 10A and 10B are omitted. Prior to actuation by a user, the actuator 104 begins in an almost fully depressed position, a portion of the skin-piercing assembly 250 is located in the lumen 234, and the housing 102 is at atmospheric pressure. The biasing member 220 is configured to not bias (e.g., not exert force on) the actuator 104 in the retraction direction R until the skin-piercing assembly 250 has been deployed. When the actuator 104 is pressed slightly in the deployment direction D, the skin-piercing assembly 250 is deployed. Once the skin-piercing assembly 250 has been deployed, the actuator 104 will be biased in the retraction direction R by the biasing member 220. As the actuator 104 is pushed in the retraction direction R, vacuum will be generated in the housing 102. In comparison to the device 100 of FIGS. 10A and 10B, the device 1100 requires significantly less force to deploy and also does not require the vent 219 (FIGS. 10A and 10B) to generate vacuum as excess gas (e.g., ambient air) is removed from the housing 102 during manufacturing. In some aspects of the present technology, the reduced force required for activation of the device 1100 can be beneficial for patients with mobility and strength issues. It can also reduce the form factor of the housing 102 (FIG. 1A) prior to deployment, which can decrease packaging costs. Further, omitting the vent 219 can reduce manufacturing costs for the device 1100 compared to the device 100.

In some embodiments, the actuator 104, the housing 102, and/or the housing 102 can include additional features designed to inhibit accidental deployment of the skin-piercing assembly 250. For example, the actuator 104 can include features that require the actuator 104 to be rotated about the axis L (FIG. 2) before it can be pressed (e.g., moved) in the deployment direction. In other embodiments, the housing 102 can include an external actuator cover that must be removed from the housing 102 before the actuator 104 can be pressed. In yet other embodiments, the housing 102 can include a button on the side of the housing 102 that must be pressed before the actuator 104 can be pressed.

In some embodiments, the housing 102 can be divided into multiple portions and one or more portions can include stored vacuums that can be used to obviate the vent 219. For example, the housing 102 can be a stepped piston, and the upper half of the stepped piston can contain a stored vacuum.

FIG. 12A is a perspective view of a bodily fluid collection device 1200 (“device 1200”) in a first position configured in accordance with embodiments of the present technology. FIGS. 12B and 12C are perspective cross-sectional views of the bodily fluid collection device 1200 of FIG. 12A in a second position and a third position, respectively, configured in accordance with embodiments of the present technology. The device 1200 can include some features that are at least generally similar in structure and function, or identical in structure and function, to the corresponding features of the devices 100 and 1100 described in detail above with reference to FIGS. 1A-11, and can operate in a generally similar or identical manner to the devices 100 and 1100.

Referring to FIGS. 12A-12C together, in the illustrated embodiment, the device 1200 includes a compressible housing 1204 and a containment structure 1202 at least partially around the compressible housing 1204. The compressible housing 1204 is operably coupled to a skin-piercing assembly 1250. In operation, the compressible housing 1204 (e.g., a top surface 1205 thereof) can be actuated to elastically deform/compress the compressible housing 1204 and drive the skin-piercing assembly 1250 toward the skin 101 (FIG. 2) of a user and cause the skin-piercing assembly 1250 to pierce the skin 101. The compressible housing 1204 defines a volume 1249 that (i) reduces in volume when compressed during actuation and (ii) increases in volume when released, and the compressible housing 1204 returns to its original shape to generate vacuum pressure in the volume 1249.

In the illustrated embodiment, the containment structure 1202 is a hard case substantially surrounding the compressible housing 1204. The top surface 1205 of the compressible housing 1204 extends beyond an upper edge 1203 of the containment structure 1202 such that the user can access and actuate the compressible housing 1204. The compressible housing 1204 comprises a flexible material (e.g., rubber) that can be deformed during operation of the device 1200 to drive the skin-piercing assembly 1250 toward the skin 101 (FIG. 2). As noted above, the material of the compressible housing 1204 can be elastically deformed such that the compressible housing 1204 returns to its original shape when force is no longer applied thereto. As such, in some embodiments, the biasing member 220 (not shown; FIG. 2) can be omitted, and a vacuum can be generated when pressure is released from the compressible housing 1204 and the compressible housing 1204 returns to its original shape. In the illustrated embodiment, the skin-piercing assembly 1250 is fixedly coupled to a center of the compressible housing 1204 (e.g., opposite a center of the top surface 1205). In some embodiments, some portions of the compressible housing 1204 can be inflexible (e.g., rigid) to make it easier to deform the other portions of the compressible housing 1204 and/or to create a flat surface where the skin-piercing assembly 1250 can be affixed.

Referring to FIG. 12A, in the illustrated embodiment the containment structure 1202 has a height H. In some embodiments, the height H of the containment structure 1202 can vary depending on the desired actuating force of the compressible housing 1204. For example, the height H can be less when the desired actuating force is less. As the height H of the containment structure 1202 varies, the amount the compressible housing 1204 must be deformed varies, and thus the actuating force of the compressible housing 1204 varies. For example, as the height H increases, the actuating force required to deform the compressible housing 1204 increases. In some aspects of the present technology, various actuating forces can be beneficial in different circumstances.

In the illustrated embodiment, the compressible housing 1204 also includes a flexible outflow port 1215. In some embodiments, the flexible outflow port 1215 can be integrally formed with the compressible housing 1204 and can improve a seal between the device 1200 (e.g., the compressible housing 1204) and the collection reservoir 106. In the illustrated embodiment, an opening 1246 of the flexible outflow port 1215 can be compressed or stretched to change a diameter of the opening 1246. In some aspects of the present technology, this can allow the outflow port 1215 to be coupled to a variety of collection reservoirs 106. For example, when the collection reservoir 106 has a relatively small opening (e.g., a smaller diameter than the opening 1246), then the outflow port 1215 can surround (e.g., on the outside) an end of the collection reservoir 106 to couple the collection reservoir 106 to the containment structure 1202. Further, when the collection reservoir 106 has a relatively large opening (e.g., a larger diameter than the opening 1246), then the outflow port 1215 can be inserted into the collection reservoir 106 (e.g., on the inside) to couple the collection reservoir 106 to the containment structure 1202. That is, the outflow port 1215 can flexibly form a seal with either the inside or the outside of the collection reservoir 106.

Referring to FIG. 12C, in the illustrated embodiment the device 1200 includes a valve 1219 (e.g., an umbrella valve) that operates to permit air to leave the volume 1249 when the compressible housing 1204 is compressed, and permits air from entering the volume 1249 when the compressible housing 1204 returns to its original position to facilitate vacuum generation in the volume 1249. In other embodiments, the compressible housing 1204 can include a slit or flap (not shown) instead of the valve 1219 in the top surface 1205 that extends through an entire thickness of the compressible housing 1204 and that operates like the valve 1219. For example, in the first position, the slit can be substantially closed such that air cannot pass through the slit. As the compressible housing 1204 moves in the deployment direction D between the first position and the second position, air can pass out of the containment structure 1202 through the slit, similar to how air is forced out of the valve 1219. More specifically, pressure on the compressible housing 1204 from the user's fingers can cause the slit to split open, allowing air to pass through the slit when the compressible housing 1204 moves in the deployment direction D (FIG. 12B). Then, as the compressible housing 1204 moves in the retraction direction R between the second and third position, the slit can become sealed to generate a vacuum within the containment structure 1202. More specifically, when the user releases pressure on the compressible housing 1204 to allow the compressible housing 1204 to move in the retraction direction R (FIG. 12C), the slit can close to inhibit air from entering the containment structure 1202. In other embodiments, other mechanisms can be used to cause the slit to open in the deployment direction D and close in the retraction direction R. In some embodiments, the slit can remain closed in the first position to keep the components within the containment structure 1202 sanitary prior to use.

Referring to FIG. 12B, a floating island 1230 includes circular walls 1233. In the illustrated embodiment, in contrast to the floating island 1230 of FIGS. 7A-7D, the circular walls 1233 extend outward toward walls of the compressible housing 1204 and the housing 102. In some aspects of the present technology, the enlarged circular walls guide the floating island 1230 and/or the skin-piercing assembly 1250 as it moves within the housing 102. More specifically, because the floating island 1230 is coupled to the compressible housing 1204 via the membrane 212 (FIG. 2), it is possible for the floating island 1230 to rotate relative to the housing 102. As such, internal surfaces of the circular walls 1233 can guide the movement of the skin-piercing assembly 1250 as the compressible housing 1204 is depressed, ensuring that the skin-piercing assembly 1250 is properly aligned with the cutting region 235 when it is actuated (e.g., perpendicular to the skin 101 (FIG. 2)).

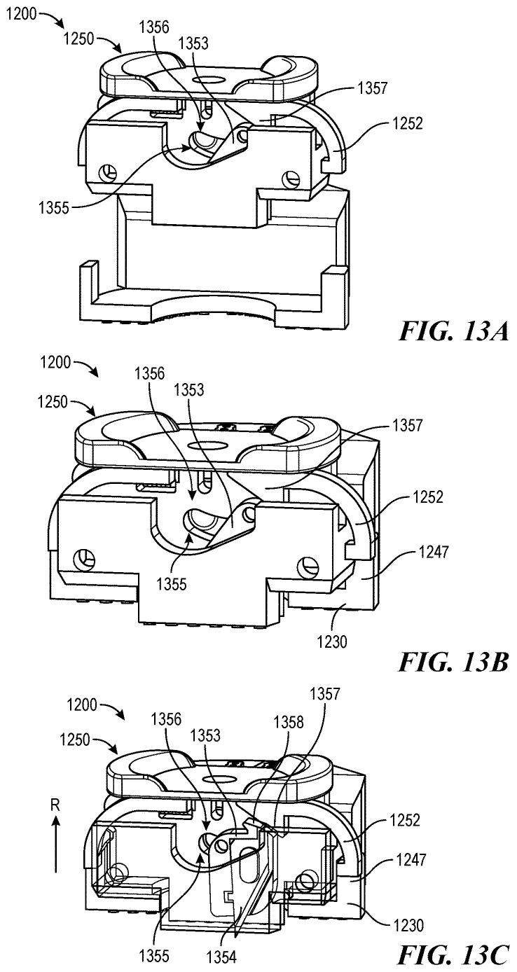

In the illustrated embodiment, the skin-piercing assembly 1250 includes a release member 1252 operably coupled to the skin-piercing feature (e.g., drive member 1353 (FIG. 13A)). More specifically, the release member 1252 can be moved to deploy the skin-piercing feature of the skin-piercing assembly 1250. Further, the floating island 1230 includes a deployment member 1247 adjacent to the circular walls 1233. During the operation of the device, the release member 1252 interfaces with the deployment member 1247 to deploy the skin-piercing feature of the skin-piercing assembly 1250, as described in greater detail below with reference to FIGS. 13A-13C.

Referring to FIG. 12A, in the first position, the compressible housing 1204 is not actuated (e.g., uncompressed) and extends out of the housing 102. As such, vacuum is not generated within the housing 102 and/or within the compressible housing 1204, and the inside of the housing 102 and the compressible housing 1204 are at atmospheric pressure. Further, the skin-piercing assembly 1250 has not been actuated.

Referring to FIG. 12B, in the second position, the compressible housing 1204 is fully depressed. More specifically, between the first position and the second position, the compressible housing 1204 is depressed in the deployment direction D by the user until the skin-piercing assembly 1250 is actuated. In the second position, the release member 1252 of the skin-piercing assembly 1250 is forced upward by the deployment member 1247 of the floating island 1230, causing the skin-piercing feature of the skin-piercing assembly 1250 to deploy and pierce the skin 101 (FIG. 2) of the patient.

Referring to FIG. 12C, in the third position, pressure is removed from the compressible housing 1204, and it returns to the shape it had in the first position, causing vacuum to be generated within the housing 102. As vacuum is generated, the floating island 1230 rises into the housing 102 along with the skin 101 (not shown) in the retraction direction R, as described in greater detail above with reference to FIG. 2. In the third position, blood can flow from the cutting region 235 to the collection reservoir 106 for collection. In some embodiments, the device 1200 can include a biasing member (e.g., the biasing member 220) to bias the compressible housing 1204 in the retraction direction R.

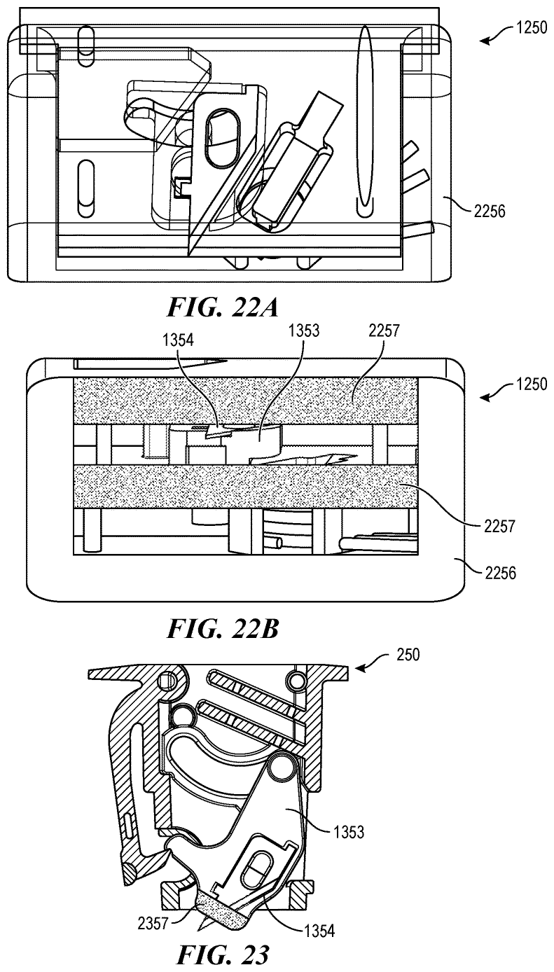

FIGS. 13A-13C are perspective views of the skin-piercing assembly 1250 of FIGS. 12B and 12C in a first position, second position, and third position, respectively, configured in accordance with embodiments of the present technology. Referring to FIGS. 13A-13C together, the skin-piercing assembly 1250 includes the release member 1252 operably coupled to a drive member 1353 that is fixedly coupled to a blade 1354 (FIG. 13C). The drive member 1353 includes a pin (not shown) that is slideably positioned within a curved slot 1355 and a biasing member (not shown) that biases the drive member 1353 from a first end 1356 of the curved slot 1355 to a second end (not shown) of the curved slot 1355. More specifically, when deployed, the pin of the drive member 1353 can slide along the curved slot 1355 from the first end 1356 to the second end due to the biasing force from the biasing member, as described in greater detail below with reference to FIG. 13C. In some embodiments, the skin-piercing assembly 1250 can include a skin guard (e.g., skin guard 2357 (FIG. 23)), described in greater detail below with reference to FIGS. 22A and 22B.

Referring to FIG. 13A, the skin-piercing assembly 1250 is in the first position, which corresponds to the first position of the bodily fluid collection device 1200 in FIG. 12A. More specifically, the compressible housing 1204 (FIG. 12A) is not depressed, and the drive member 1353 of the skin-piercing assembly 1250 is not deployed. In the first position, the pin of the drive member 1353 is positioned in the first end 1356 of the curved slot 1355. Further, a first engagement feature 1357 of the release member 1252 is coupled to a second engagement feature 1358 (FIG. 13C) of the drive member 1353. More specifically, the first engagement feature 1357 is engaged with the second engagement feature 1358 to inhibit movement of the drive member 1353 from the first end 1356 to the second end of the curved slot 1355.

Referring to FIG. 13B, the skin-piercing assembly 1250 is in a second position, which corresponds to the compressible housing 1204 of the skin-piercing assembly 1250 being almost fully depressed. As such, the release member 1252 is in the first position described above and is also contacting the deployment member 1247 of the floating island 1230; however, not enough pressure has been applied to the compressible housing 1204 for the deployment member 1247 to move the release member 1252. Accordingly, the drive member 1353 of the skin-piercing assembly 1250 is not deployed, and the first engagement feature 1357 is in the first position operably coupled to the second engagement feature 1358 to inhibit movement of the drive member 1353 from the first end 1356 to the second end of the curved slot 1355.

Referring to FIG. 13C, the skin-piercing assembly 1250 is in a third position corresponding to the second position of the bodily fluid collection device 1200 in FIG. 12B. More specifically, the compressible housing 1204 (FIG. 12B) is fully depressed, and the drive member 1353 of the skin-piercing assembly 1250 is deployed. In the third position, the deployment member 1247 of the floating island 1230 exerts sufficient force on the release member 1252 to disengage the first engagement feature 1357 from the second engagement feature 1358 of the drive member 1353. In some embodiments, this can be accomplished by the release member 1252 being rotated and/or displaced such that the first engagement feature 1357 and the second engagement feature 1358 become misaligned and/or the like. In the illustrated embodiment, once the first engagement feature 1357 disengages from the second engagement feature 1358, the biasing member (not shown) operably coupled to the drive member 1353 biases the drive member 1353 from the first end 1356 of the curved slot 1355 to the second end (obscured). More specifically, due to the curve in the curved slot 1355, the blade 1354 follows a cycloidal path (e.g., cam motion) that creates a short but relatively deep cut in the skin 101 (not shown) of the patient, which can enhance blood flow from the cut. In other aspects of the present technology, the curved slot 1355 allows the blade 1354 to be retracted upward (e.g., in the retraction direction R) after the skin has been pierced, reducing the risk of additional abrasions. In other embodiments, the shape of the curved slot 1355 can be varied depending on the desired cut shape. For example, the curved slot 1355 can be straight, triangular, sinusoidal, or other shapes.

In the illustrated embodiment, once the first engagement feature 1357 disengages from the second engagement feature 1358, it cannot re-engage with the second engagement feature 1358. In some aspects of the present technology, this inhibits the skin-piercing assembly 1250 from being deployed a second time, which can reduce the risk of accidental abrasions and/or the spread of blood-borne illnesses if multiple users attempt to use the same device 1200. In other embodiments with multiple skin-piercing features (e.g., blades 1354), multiple release members 1252 can be used to deploy each skin-piercing feature, or each skin-piercing feature can be operably coupled to a single release member 1252. In some embodiments, multiple skin-piercing features can be deployed sequentially, either after a single trigger event (e.g., a predetermined pressure exerted on the compressible housing 1204), as different degrees of pressure are applied to the compressible housing 1204, and/or the like.

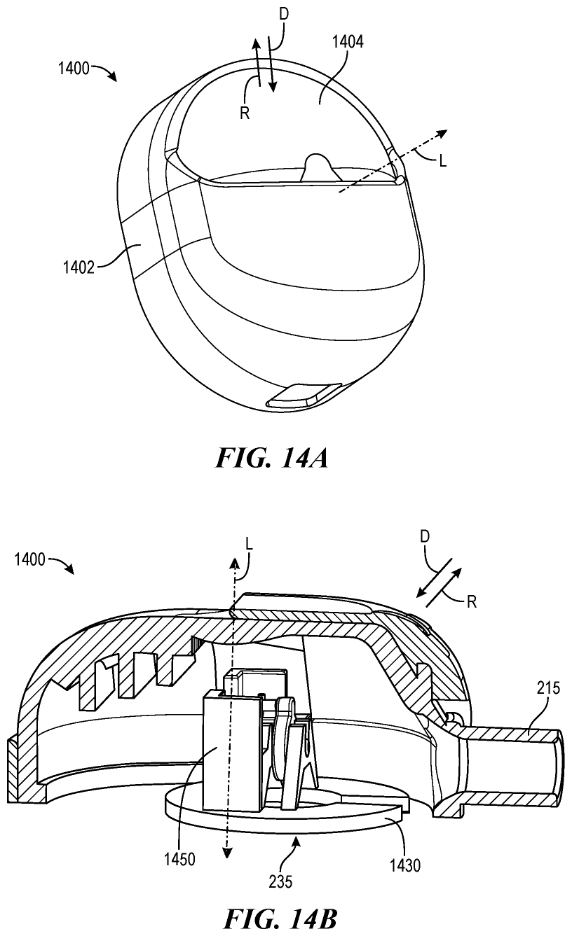

FIG. 14A is a perspective view of a bodily fluid collection device 1400 (“device 1400”) configured in accordance with embodiments of the present technology. FIG. 14B is a partially cross-sectional perspective view of the bodily fluid collection device 1400 of FIG. 14A configured in accordance with embodiments of the present technology. The device 1400 can include some features that are at least generally similar in structure and function, or identical in structure and function, to the corresponding features of the devices 100, 1100, and 1200 described in detail above with reference to FIGS. 1A-13C, and can operate in a generally similar or identical manner to the devices 100, 1100, and 1200. For example, the device 1400 includes a containment structure 1402 with the outflow port 215 (FIG. 14B) and a compressible housing 1404 operably coupled to a skin-piercing assembly 1450 (FIG. 14B).

Referring to FIGS. 14A and 14B together, the compressible housing 1404 is a flexible material, similar to the compressible housing 1204 described in greater detail above with reference to FIGS. 12A-12C. In contrast to the containment structure 1402, a center of the compressible housing 1404 is offset from the axis L of the containment structure 1402. As such, when depressed in the deployment direction D and released to move in the retraction direction R, the compressible housing 1404 moves at an angle relative to the axis L. Accordingly, features of the skin-piercing assembly 1450 and/or the floating island 1430 (FIG. 14B) can be modified such that the skin-piercing assembly 1450 aligns with the floating island 1430 and the cutting region 235 when the compressible housing 1404 is depressed. For example, circular walls (e.g., circular walls 233 (FIG. 2)) of the floating island 1430 and/or features of the skin-piercing assembly 1450 can be positioned at an angle relative to the axis L to direct the skin-piercing assembly 1450 to an area directly above the cutting region 235 when the containment structure 1402 is depressed. In some aspects of the present technology, placing the compressible housing 1404 off-center from the containment structure 1402 can enhance the ergonomics of the device 1400, which can be beneficial when the device 1400 is used by disabled users or users with limited mobility.

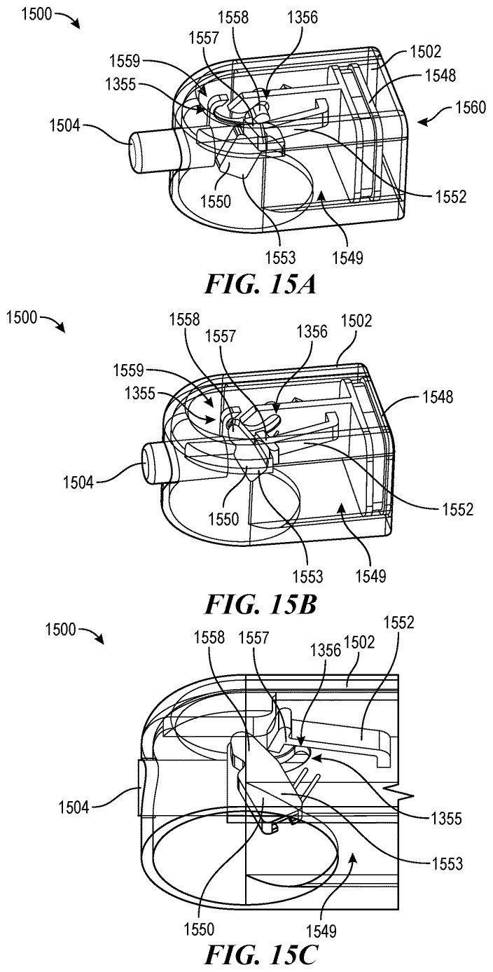

FIG. 15A is a transparent perspective view of a bodily fluid collection device 1500 (“device 1500”) in a first position in accordance with additional embodiments of the present technology. FIGS. 15B and 15C are transparent perspective views of the bodily fluid collection device 1500 of FIG. 15A in a second position in accordance with additional embodiments of the present technology. The device 1500 can include some features that are at least generally similar in structure and function, or identical in structure and function, to the corresponding features of the devices 100, 1100, 1200, and 1400 described in detail above with reference to FIGS. 1A-14B, and can operate in a generally similar or identical manner to the devices 100, 1100, 1200, and 1400. For example, the device 1500 includes a housing 1502 and an actuator 1504 operably coupled to a skin-piercing assembly 1550 having a drive member 1553 operably coupled to a release member 1552.

Referring to FIGS. 15A-15C together, the actuator 1504 is fixedly coupled to a plunger head 1548. The plunger head 1548 is sealingly and slidingly engaged with an internal surface of the housing 1502 such that when the housing 1502 is sealingly engaged with the skin 101 (not shown) of the patient, there is a first sealed volume 1549 within the housing 102. In the illustrated embodiment, the housing 1502 includes an opening 1560 to the atmosphere (e.g., not sealed), so that pressure does not build in the volume opposite the first sealed volume 1594 as the plunger head 1548 moves throughout the housing 1502.

In the illustrated embodiment, the actuator 1504 is fixedly coupled to the release member 1552, and the release member 1552 can elastically deform under pressure relative to the actuator 1504. The release member 1552 includes a first engagement feature 1557, and the drive member 1553 includes a second engagement feature 1558. Further, the drive member 1553 includes a pin (not shown) slidably positioned within the curved slot 1355, and the drive member 1553 is biased, via a biasing member (not shown), from the first end 1356 to a second end 1559 of the curved slot 1355. The first engagement feature 1557 and the second engagement feature 1558 are operably coupled such that the release member 1552 inhibits movement of the drive member 1553. More specifically, when the first engagement feature 1557 and the second engagement feature 1558 are engaged, the pin of the drive member 1553 is restrained within the first end 1356 of the curved slot 1355.

Referring to FIG. 15A, in the first position, the actuator 1504 is not actuated (e.g., not depressed, uncompressed) and extends out of the housing 1502. Accordingly, vacuum has not been generated within the housing 1502, and the inside of the housing 1502 is at atmospheric pressure. Further, the skin-piercing assembly 1250 has not been actuated, and the first engagement feature 1557 is engaged with the second engagement feature 1558 to restrain movement of the drive member 1553. More specifically, the pin of the drive member 1553 is positioned within the first end 1356 of the curved slot 1355.

Referring to FIGS. 15B and 15C together, in the second position, the actuator 1504 is actuated (e.g., depressed, compressed) and is pushed substantially into the housing 1502, causing several reactions within the housing 1502. First, the plunger head 1548 is pushed through the housing 1502, expanding the first sealed volume 1549 and generating vacuum within the first sealed volume 1549. Second, pressing the actuator 1504 places pressure on the first engagement feature 1557, causing it to deform and release the second engagement feature 1558. As a result, the drive member 1553 is released and moves in a curved arc from the first end 1356 to the second end 1559, as described in greater detail above with reference to FIG. 13C. Like the device 1200, once the first engagement feature 1557 releases the second engagement feature 1558, they cannot be re-engaged, which inhibits a second deployment of the skin-piercing assembly 1550. In other embodiments, the release member 1552 and/or components of the skin-piercing assembly 1550 can be separated from (e.g., not directly coupled to) the actuator 1504.

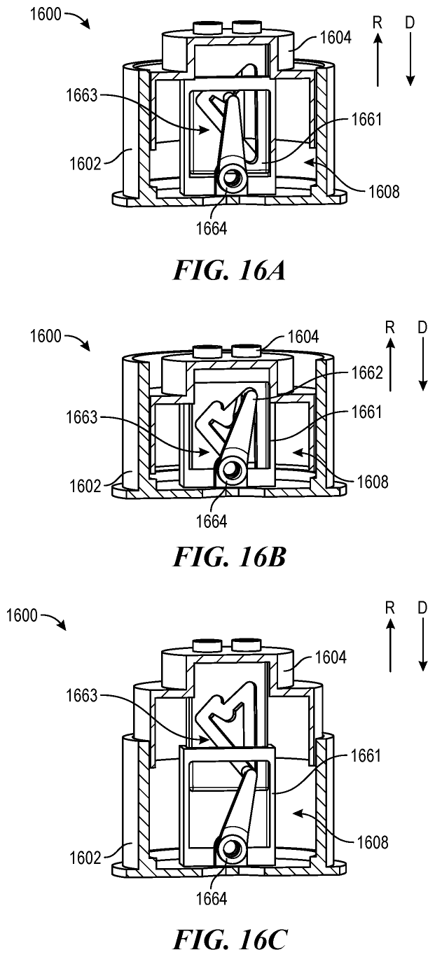

FIGS. 16A-16C are cross-sectional side views of a bodily fluid collection device 1600 (“device 1600”) in a first position, second position, and third position, respectively, configured in accordance with embodiments of the present technology. The device 1600 can include some features that are at least generally similar in structure and function, or identical in structure and function, to the corresponding features of the devices 100, 1100, 1200, 1400, and 1500 described in detail above with reference to FIGS. 1A-15C, and can operate in a generally similar or identical manner to the devices 100, 1100, 1200, 1400, and 1500. For example, the device 1600 includes a housing 1602 and an actuator 1604 operably coupled to a skin-piercing assembly (not shown).

Referring to FIGS. 16A-16C together, the actuator 1604 is operably coupled to a vacuum source 1608. The vacuum source 1608 includes a cardioid slot mechanism 1661 with a rotatable pin 1662 and a continuous slot 1663. The rotatable pin 1662 is positioned partially within the continuous slot 1663 and rotates about a fixed end 1664 of the rotatable pin 1662 while a portion of the rotatable pin 1662 remains in the continuous slot 1663. The cardioid slot mechanism 1661 also includes a biasing member (not shown) that biases the continuous slot 1663 in the retraction direction R.

Referring to FIG. 16A, in the first position, the actuator 1604 is not actuated, and the rotatable pin 1662 is held in a first position within the continuous slot 1663 by the biasing member. Similar to the device 1100 described in FIG. 11, the actuator 1604 begins almost fully compressed. More specifically, the actuator 1604 can be moved farther in the retraction direction R than in the deployment direction D. In some aspects of the present technology, when the actuator 1604 is almost fully compressed in the first position (e.g., before deployment of the skin-piercing assembly, before use), the form factor of the device 1600 can be reduced, which can reduce shipping costs of the device 1600.

Referring to FIG. 16B, in the second position, the actuator 1604 is fully compressed after being pushed in the deployment direction D by the user, and the rotatable pin 1662 is moved to a second position within the continuous slot 1663 due to the movement of the actuator 1604. More specifically, in the second position, the rotatable pin 1662 is at an apex of the continuous slot 1663. In the second position, the skin-piercing assembly is also deployed.

Referring to FIG. 16C, in the third position, the actuator 1604 is released, causing the biasing member to bias the actuator 1604 and the continuous slot 1663 in the retraction direction R until the rotatable pin 1662 reaches a bottom portion of the continuous slot 1663, inhibiting further movement of the actuator 1604 and the continuous slot 1663 in the retraction direction R. As the actuator 1604 moves in the retraction direction R, vacuum is generated, as described in greater detail above with reference to FIG. 11. In some embodiments, if the actuator 1604 is depressed again in the deployment direction D, the rotatable pin 1662 can move up the left side of the continuous slot 1663 where it can be restrained to inhibit additional attempts to deploy the skin-piercing assembly.

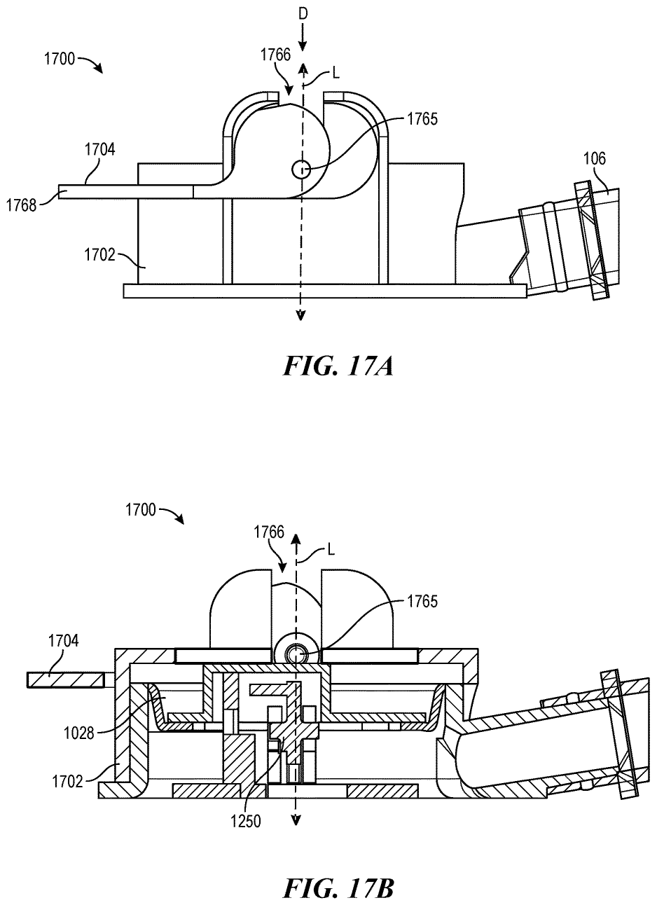

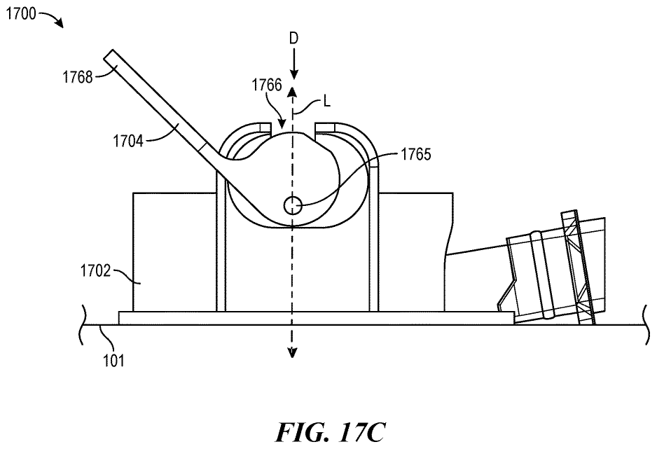

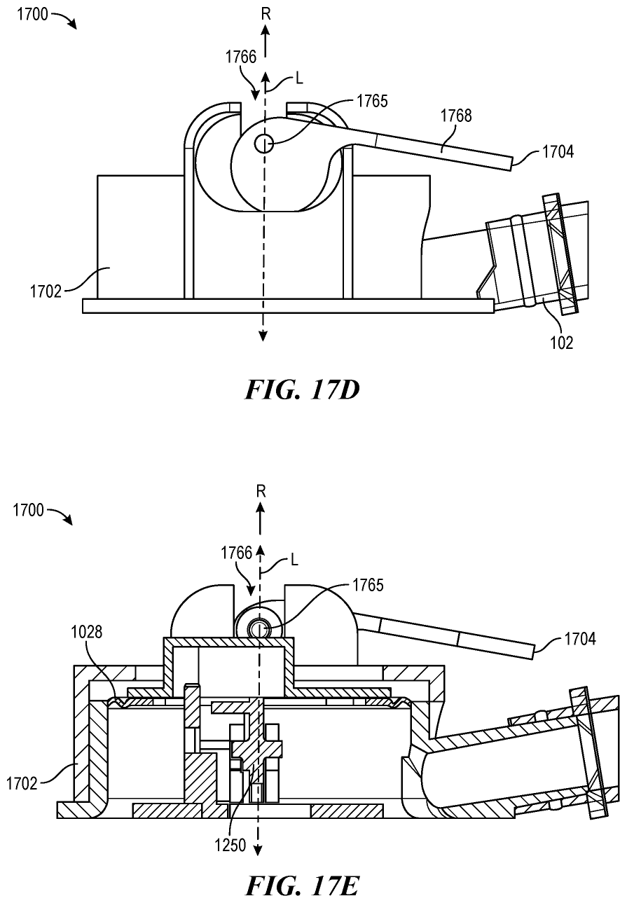

FIG. 17A is a side view of a bodily fluid collection device 1700 (“device 1700”) in a first position configured in accordance with embodiments of the present technology. FIG. 17B is a cross-sectional side view of the bodily fluid collection device 1700 of FIG. 17A in the first position configured in accordance with embodiments of the present technology. FIG. 17C is a side view of the bodily fluid collection device 1700 of FIG. 17A in a second position configured in accordance with embodiments of the present technology. FIG. 17D is a side view of the bodily fluid collection device 1700 of FIG. 17A in a third position configured in accordance with embodiments of the present technology. FIG. 17E is a cross-sectional side view of the bodily fluid collection device 1700 of FIG. 17A in the third position configured in accordance with embodiments of the present technology. The device 1700 can include some features that are at least generally similar in structure and function, or identical in structure and function, to the corresponding features of the devices 100, 1100, 1200, 1400, 1500, and 1600 described in detail above with reference to FIGS. 1A-16C, and can operate in a generally similar or identical manner to the devices 100, 1100, 1200, 1400, 1500, and 1600. For example, the device 1700 includes a housing 1702 and an actuator 1704 operably coupled to the skin-piercing assembly 1250 and the flexible seal 1028.

Referring to FIGS. 17A-17E together, the actuator 1704 is a lever that rotates about an axis that moves in a linear cam motion. More specifically, as the actuator 1704 is rotated (e.g., from the first position to the third position), a pin 1765 of the actuator 1704 moves along the axis L within a channel 1766 of the housing 1702.

Referring to FIG. 17A, in the first position, an arm 1768 of the actuator 1704 extends in a direction opposite the collection reservoir 106 relative to the housing 1702. In the illustrated embodiment, the arm 1768 is U-shaped and extends circumferentially around the housing 1702 such that the pin 1765 extends from one end of the arm 1768 to an opposite end of the arm 1768 through the channel 1766. In other embodiments, the arm 1768 can be L-shaped and/or the like. In the illustrated embodiment, the arm 1768 is perpendicular to the axis L in the first position. In other embodiments, the arm 1768 can be at a different angle relative to the axis L (e.g., 80 degrees, 70 degrees, 60 degrees) to reduce the amount of force required to actuate the actuator 1704.

Referring to FIG. 17B, in the first position, the flexible seal 1028 is relaxed (e.g., loose, hanging), and the inside of the housing 1702 is at atmospheric pressure. Additionally, the skin-piercing assembly 1250 is not deployed (e.g., undeployed). As described in greater detail below with reference to FIG. 17E, the flexible seal 1028 can be moved by actuating the actuator 1704 (e.g., moving it from the first position to the third position).

Referring to FIG. 17C, in the second position, the arm 1768 of the actuator 1704 is rotated about the pin 1765 so that the arm 1768 extends upward (e.g., away from the housing 1702). Further, the pin 1765 is moved in the deployment direction D along the axis L within the channel 1766. In the illustrated embodiment, the motion of actuator 1704 from the first position to the second position causes the skin-piercing assembly 1250 (FIG. 17B) to deploy and cut the skin 101 of the patient. In other embodiments, the skin-piercing assembly 1250 can be deployed when the arm 1768 is at various angles relative to the longitudinal axis L, such as 0 degrees, +/−10 degrees, +/−20 degrees, +/−30 degrees, or any angle therebetween.

Referring to FIG. 17D, in the third position, the arm 1768 of the actuator 1704 is rotated about the pin 1765 so that the arm 1768 extends generally perpendicular to the axis L adjacent to the collection reservoir 106. Further, the pin 1765 moves in the retraction direction R along the axis L within the channel 1766. Referring to FIG. 17E, the movement of the actuator 1704 in the retraction direction R from the second position to the third position causes the actuator 1704 to retract the flexible seal 1028 in the retraction direction R, generating vacuum within the housing 1702. In some embodiments, moving the actuator 1704 to the third position can also retract the skin-piercing assembly 1250 in the retraction direction R to reduce the risk of accidental abrasions from the blade 1354 (FIG. 13C). In some embodiments, the skin-piercing assembly 1250 can be deployed in the third position, and the second position can be omitted.

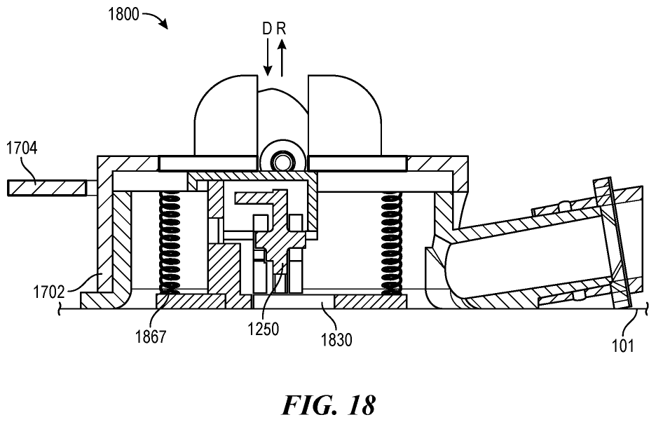

FIG. 18 is a cross-sectional side view of a bodily fluid collection device 1800 (“device 1800”) in accordance with additional embodiments of the present technology. The device 1800 can include some features that are at least generally similar in structure and function, or identical in structure and function, to the corresponding features of the devices 100, 1100, 1200, 1400, 1500, 1600 and 1700 described in detail above with reference to FIGS. 1A-17E, and can operate in a generally similar or identical manner to the devices 100, 1100, 1200, 1400, 1500, 1600, and 1700. For example, the device 1800 includes the housing 1702 and the actuator 1704 operably coupled to the skin-piercing assembly 1250.

In the illustrated embodiment, the actuator 1704 is coupled directly to a floating island 1830 via a biasing member 1867 (e.g., springs) such that movement of the actuator 1704 causes movement of the floating island 1830. For example, when the actuator 1704 is moved between the first position and the third position, as described in greater detail above with reference to FIGS. 17A-17E, the actuator 1704 can raise the floating island 1830 in the retraction direction R via the biasing member 1867. In some aspects of the present technology, this can obviate the vacuum source (e.g., vacuum source 218), as the floating island 1830 and the skin 101 are raised via movement of the actuator 1704 rather than the generation of vacuum pressure.

In other embodiments, the floating island 1830 can be fixedly coupled to the actuator 1704 such that it moves a predetermined distance in the retraction direction R during deployment. In some aspects of the present technology, using biasing members 1867, like springs, to move the floating island 1830 can reduce the risk of breaking the skin 101 and/or breaking the seal between the skin 101 and the membrane 212 (FIG. 2). For example, skin 101 that resists the movement of the floating island 1830 can pull the biasing member 1867 in the deployment direction D, reducing the movement of the floating island 1830 in the retraction direction R and, accordingly, the distance the skin 101 is pulled into the housing 1702. In contrast, if inflexible biasing members are used, the skin 101 might rip near an edge of the floating island 1830 when drawn too far into the housing 1702, causing unnecessary bleeding, and/or the seal between the skin 101 and the floating island 1830 may break, causing the collection of bodily fluid to be disrupted.

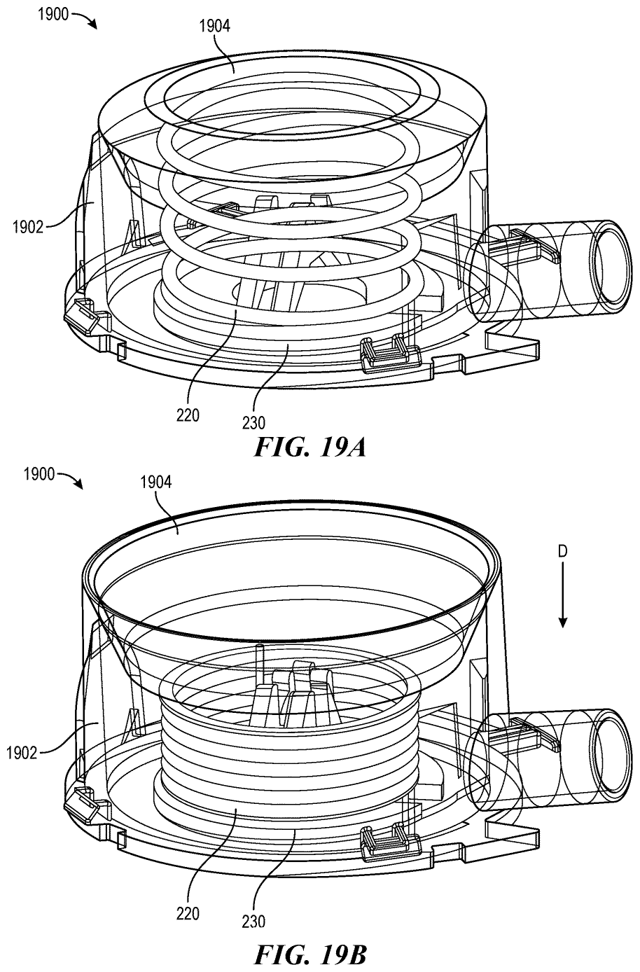

FIGS. 19A and 19B are partially transparent perspective views of a bodily fluid collection device 1900 (“device 1900”) in a first position and a second position, respectively, in accordance with additional embodiments of the present technology. The device 1900 can include some features that are at least generally similar in structure and function, or identical in structure and function, to the corresponding features of the devices 100, 1100, 1200, 1400, 1500, 1600, 1700, and 1800 described in detail above with reference to FIGS. 1A-18, and can operate in a generally similar or identical manner to the devices 100, 1100, 1200, 1400, 1500, 1600, 1700, and 1800. For example, the device 1900 includes a housing 1902 and an actuator 1904 operably coupled to the skin-piercing assembly 1250 (not shown).

Referring to FIGS. 19A and 19B together, the actuator 1904 is directly coupled to the biasing member 220 (e.g., a spring) and the biasing member 220 is directly coupled to the floating island 230. Referring to FIG. 19A, in the first position, the actuator 1904 is not actuated (e.g., not depressed). Further, the biasing member 220 is not stretched or compressed, and, as such, the actuator 1904 and the floating island 230 are static in the first position. More specifically, no force is being applied to the floating island 230 via the biasing member 220 or the actuator 1904.

Referring to FIG. 19B, in the second position, the actuator 1904 is actuated (e.g., depressed, compressed), compressing the biasing member 220 in the deployment direction D. Similarly, the biasing member 220 exerts a biasing force in the deployment direction D on the floating island 230 due to the compression of the biasing member 220. In some aspects of the present technology, putting compressive force on the skin 101 (FIG. 2) of the patient can have similar effects to applying vacuum to the skin 101, which are described in greater detail below with reference to FIGS. 24A-24D. In other aspects of the present technology, utilizing compressing forces can obviate vacuum sources (e.g., vacuum source 218 (FIG. 2)). In other embodiments, combinations of compressive and vacuum forces can be utilized in the device 1900. In some embodiments, the skin-piercing assembly 1250 can be deployed in the second position.

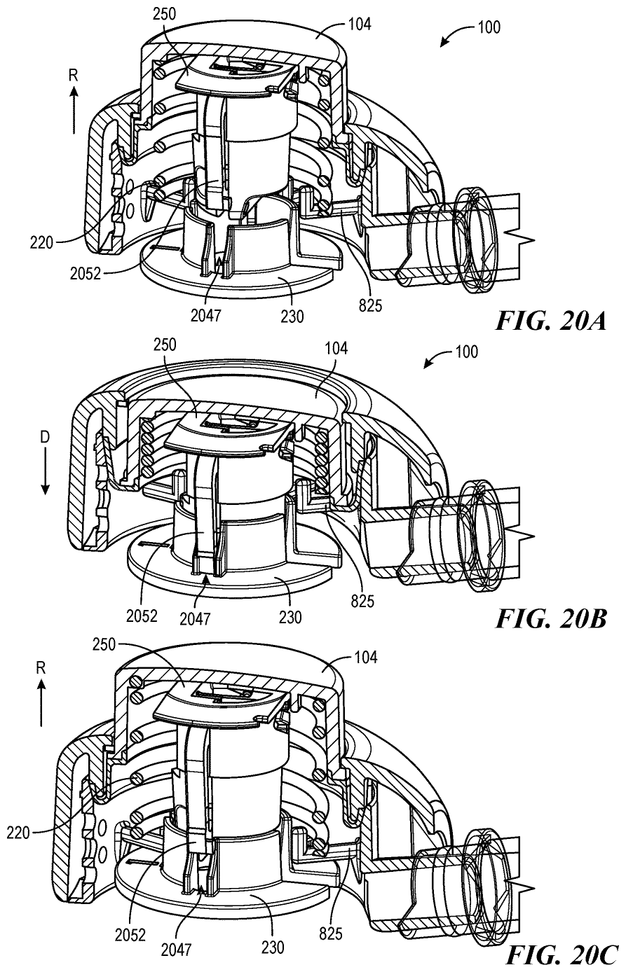

FIGS. 20A-20C are partially cross-sectional perspective views of the bodily fluid collection device 100 in a first position, a second position, and a third position, respectively, configured in accordance with embodiments of the present technology. Referring to FIGS. 20A-20C together, the skin-piercing assembly 250 includes a release member 2052, and the floating island 230 includes a deployment channel 2047 operably couplable with the release member 2052. In the illustrated embodiment, the retention arms 825 have a reduced profile compared to the retention arms 825 in FIGS. 8A and 8B.

Referring to FIG. 20A, in the first position, the actuator 104 is uncompressed, and the skin-piercing assembly 250 is not deployed. Additionally, the biasing member 220 is biasing the actuator 104 in the retraction direction R. Referring to FIG. 20B, in the second position, the actuator 104 is compressed in the deployment direction D, causing the release member 2052 of the skin-piercing assembly 250 to contact the edges of the deployment channel 2047 of the floating island 230. When the release member 2052 interfaces with the deployment channel 2047, the release member 2052 is diverted outward, causing it to release a drive member (e.g., drive member 1353 (FIG. 13C)) so that the skin-piercing assembly 250 deploys and cuts the skin 101 (FIG. 2) of the patient. Referring to FIG. 20C, the actuator 104 is released and biased in the retraction direction R by the biasing member 220, generating vacuum in the housing 102 as described in greater detail above with reference to FIGS. 10A and 10B.

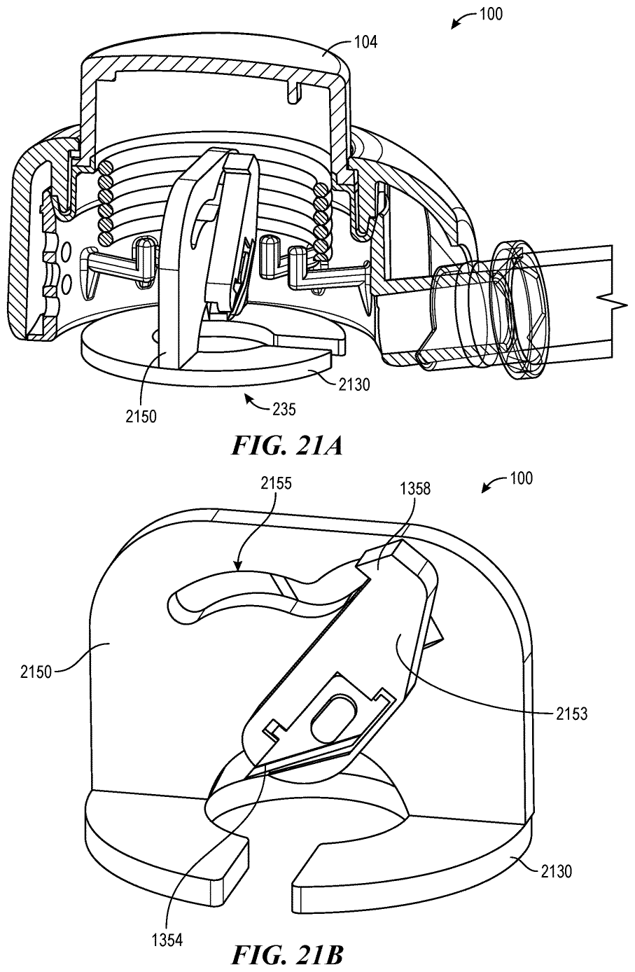

FIG. 21A is a partially cross-sectional perspective view of the bodily fluid collection device 100 configured in accordance with embodiments of the present technology. In the illustrated embodiment, the bodily fluid collection device 100 includes a floating island 2130 and a skin-piercing assembly 2150 fixedly coupled to the floating island 2130 rather than the actuator 104. Similar to the device 100 of FIG. 2, actuation of the actuator 104 causes the skin-piercing assembly 2150 to deploy. In some aspects of the present technology, combining the floating island 2130 with the skin-piercing assembly 2150 can make it easier to align the skin-piercing assembly with the cutting region 235 during operations of the device 100.

FIG. 21B is a perspective view of the floating island 2130 of the bodily fluid collection device 100 of FIG. 21A configured in accordance with embodiments of the present technology. The floating island 2130 and the skin-piercing assembly 2150 can include some features that are at least generally similar in structure and function, or identical in structure and function, to the corresponding features of the floating islands 230, 1230, and 1430 and the skin-piercing assemblies 250, 1250, 1450, and 1550, respectively, described in detail above with reference to FIGS. 1A-20C, and can operate in a generally similar or identical manner to the floating islands 230, 1230, and 1430 and the skin-piercing assemblies 250, 1250, 1450, and 1550, respectively. For example, the skin-piercing assembly 2150 includes a drive member 2153 including the blade 1354 and the second engagement feature 1358. Further, a pin (not shown) of the drive member 2153 is positioned within a curved slot 2155.

FIG. 22A is a partially transparent side view of the skin-piercing assembly 1250 of FIGS. 13A-13C configured in accordance with embodiments of the present technology. In the illustrated embodiment, the skin-piercing assembly 1250 includes a housing 2256 surrounding components of the skin-piercing assembly 1250. FIG. 22B is a bottom view of the skin-piercing assembly 1250 of FIG. 22B configured in accordance with embodiments of the present technology. In the illustrated embodiment, the housing 2256 includes skin guards 2257 on either side of the drive member 1353. In other embodiments, skin guards 2257 can be placed on one or more sides of the housing 2256. In some aspects of the present technology, the skin guards 2257 can help shape the skin prior to deployment of the blade 1354 to ensure a proper wound shape. For example, in the illustrated embodiment, before the blade 1354 is deployed, the housing 2256 contacts the skin 101 (not shown), and the skin guards 2257 shape the skin 101 (e.g., compress it) to position the skin 101 for the deployment of the blade 1354, which can help ensure a proper wound shape after deployment of the blade 1354.

FIG. 23 is a cross-sectional side view of the skin-piercing assembly 250 of FIG. 2 configured in accordance with embodiments of the present technology. In the illustrated embodiment, a skin guard 2357 is fixedly coupled to an end of the drive member 1353 adjacent to the blade 1354. During the deployment of the blade 1354, the skin guard 2357 can shape the skin 101 (not shown) as the blade 1354 sweeps across the skin 101, helping shape the skin 101 for a proper wound shape. In other embodiments, the skin guard 2357 can be positioned on the floating island 230 (FIG. 2).

FIGS. 24A-24D are charts of data comparing configurations of the bodily fluid collection devices of FIGS. 4A-6B, 6D, and 6E configured in accordance with embodiments of the present technology. With reference to FIGS. 24A-24D, the modified whisker plots include three lines parallel to the x-axis of the charts. The bottom line represents the bottom quartile of results, the middle line represents the mean result, and the top line represents the upper quartile of results.

FIG. 24A is a modified whisker plot of the bodily fluid flow rates of the devices of FIGS. 6A, 6B, 6D, and 6E. With reference to FIG. 24A, the x-axis corresponds to the housings 102 of FIGS. 6A, 6B, 6D, and 6E accordingly: (i) “14 mm” corresponds to housing 102 of FIG. 6D, (ii) “20 mm” corresponds to housing 102 of FIG. 6E, (iii) “30 mm” corresponds to housing 102 of FIG. 6A, and (iv) “45/55 mm” corresponds to two sizes of the housing 102 of FIG. 6B. FIG. 24A illustrates that in devices 100 without floating islands 230 (FIG. 2), bodily fluid flow rates decrease as vacuum areas (e.g., areas having vacuum diameters D_{V} (FIG. 3)) increase.

FIG. 24B is a modified whisker plot of the bodily fluid flow rates of the devices of FIGS. 5A-5C and additional devices. With reference to FIG. 24B, the x-axis corresponds to devices 100 of FIGS. 5A-5C accordingly: (i) “30 mm/37 kPa” corresponds to device 100 of FIG. 5A, (ii) “45 mm/35 kPa” corresponds to device 100 of FIG. 5B, and (iii) “Oblong/30 kPa” corresponds to device 100 of FIG. 5C. The additional entries “55 mm/30 kPa” and “55 mm/12 kPa” correspond to devices similar to the device 100 of FIG. 5A, however, these devices have a uniform vacuum diameter D_{V} (FIG. 3) equal to 55 mm. The second portion of the labels on the x-axis of the chart of 24B denotes the amount of pressure that was applied for each set of samples. For example, “30 mm/37 kPa” indicates 37 kPa was applied during sampling. FIG. 24B illustrates that in devices 100 with floating islands 230 (e.g., device 100 of FIG. 2), bodily fluid flow rates increase as vacuum areas (e.g., areas vacuum diameters D_{V}) increase, in contrast with devices without floating islands 230 (e.g., housings 502 of FIGS. 6A-6E).

FIG. 24C is a modified whisker plot of the bodily fluid flow rates of the devices of FIGS. 4A-4D. With reference to FIG. 24C, the x-axis corresponds to devices 100 of FIGS. 4A-4D accordingly: (i) “Half Moon Island” corresponds to device 100 of FIG. 4A, (ii) “Small Island” corresponds to device 100 of FIG. 4B, (iii) “Big Island” corresponds to device 100 of FIG. 4C, and (iv) “Flat Top Island” corresponds to device 100 of FIG. 4D. FIG. 24C illustrates that bodily fluid flow rates increase as island outer diameters D_{IO} (FIG. 3) increase.

FIG. 24D is a modified whisker plot of the bodily fluid flow rates of a variety of cut configurations. In the illustrated chart, “5 mm, 11.6 mm/37 kPA” corresponds to a device 100 (FIG. 1A) having a vacuum diameter D_{V} (FIG. 3) of 11.6 mm; two parallel cuts 951 (FIG. 9D) with a length of 5 mm; and 37 kPa of negative pressure applied to the skin 101 (FIG. 1B). Similarly, “5 mm, 30 mm/30 kPa” corresponds to a device 100 having a vacuum diameter D_{V} of 30 mm; two parallel cuts 951 with a length of 5 mm; and 30 kPa of negative pressure applied to the skin 101. Finally, “7.3 mm, 30 mm/30 kPa” corresponds to a device 100 having a vacuum diameter D_{V} of 30 mm; two parallel cuts 951 with a length of 7.3 mm; and 30 kPa of negative pressure applied to the skin 101. FIG. 24D illustrates that increasing vacuum area (e.g., areas having vacuum diameters D_{V}) and the size of cuts 951 increases bodily fluid flow rates.

FIG. 25 is a perspective view of a bodily fluid collection device 2500 in a first position (e.g., a pre-loaded position, a pre-actuated position) in accordance with additional embodiments of the present technology. The device 2500 can include some features that are at least generally similar in structure and function, or identical in structure and function, to the corresponding features of any of the bodily fluid collection devices described in detail above with reference to FIGS. 1A-24D, and can operate in a generally similar or identical manner to any of those devices.

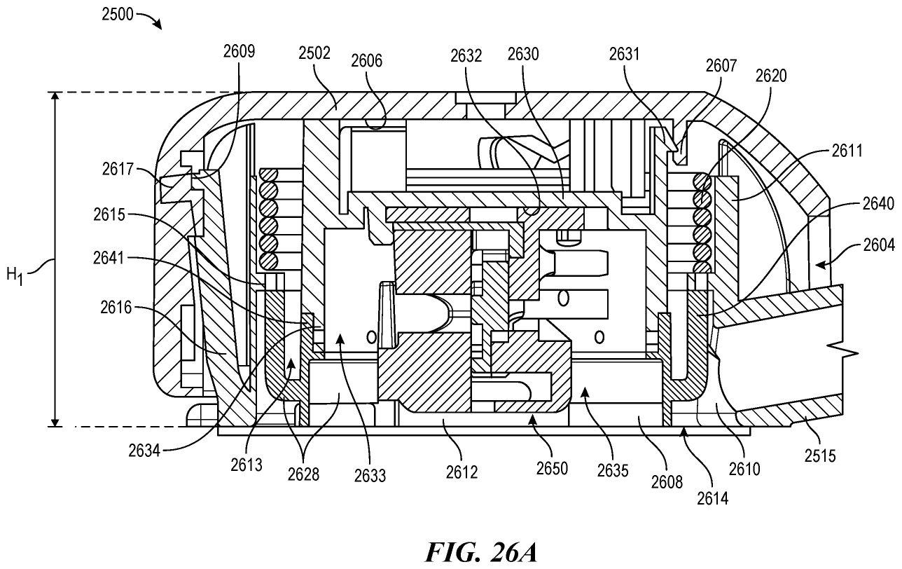

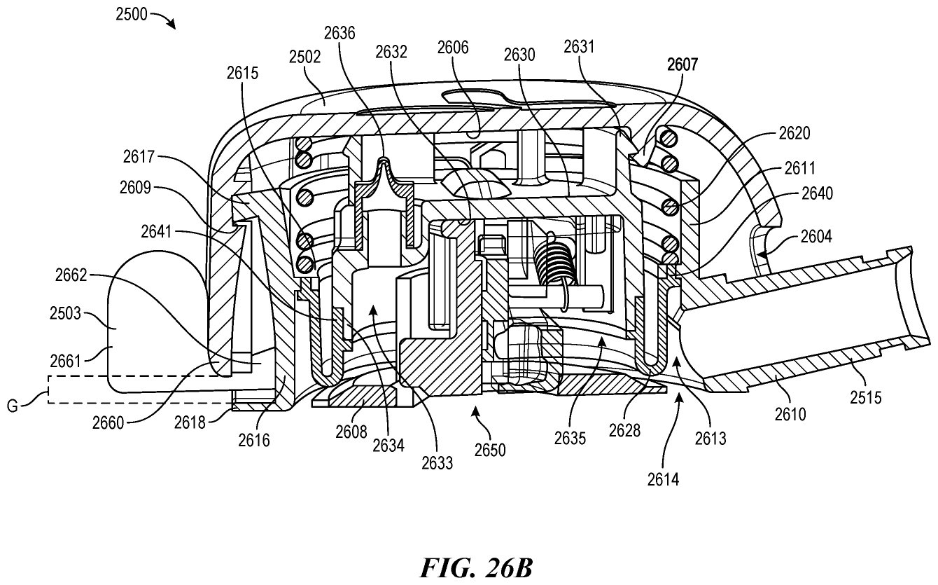

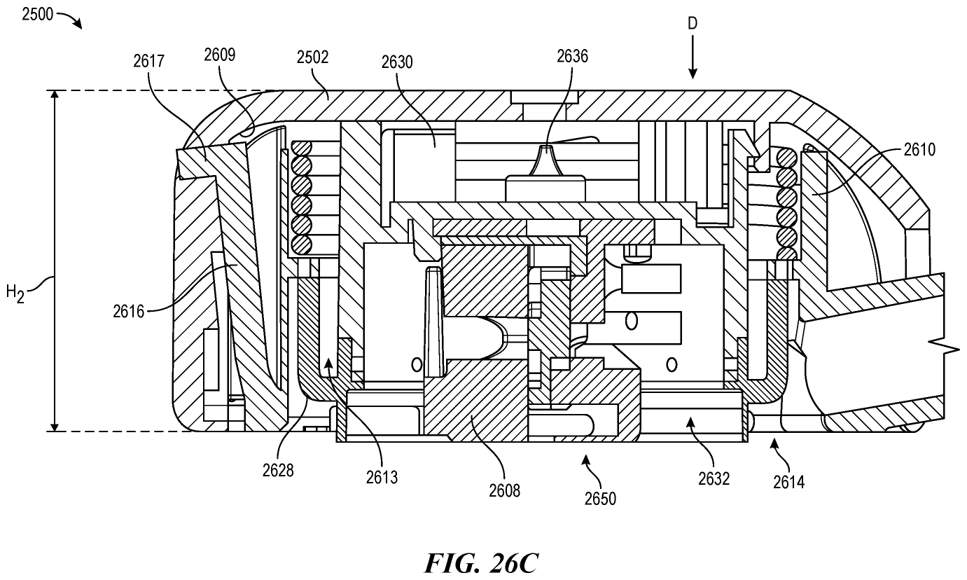

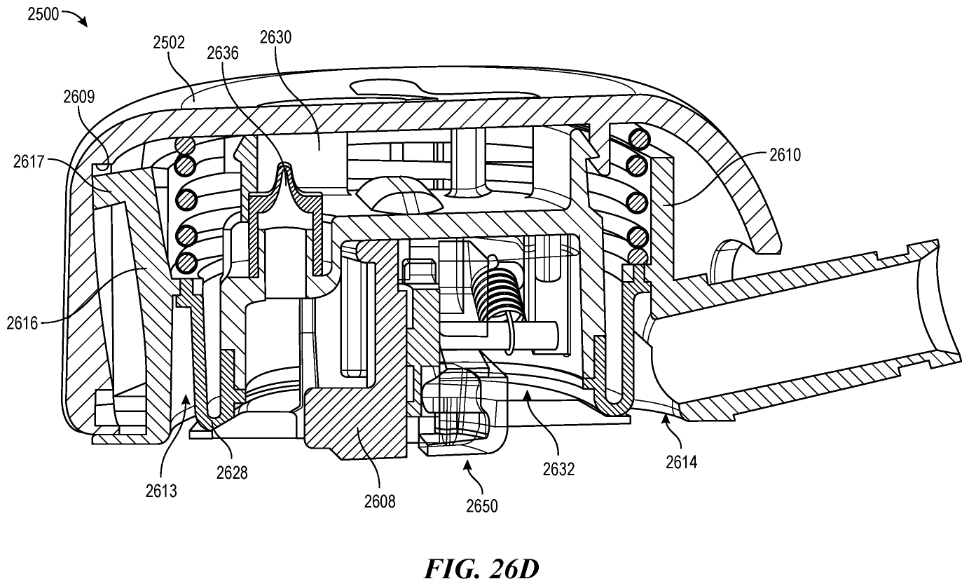

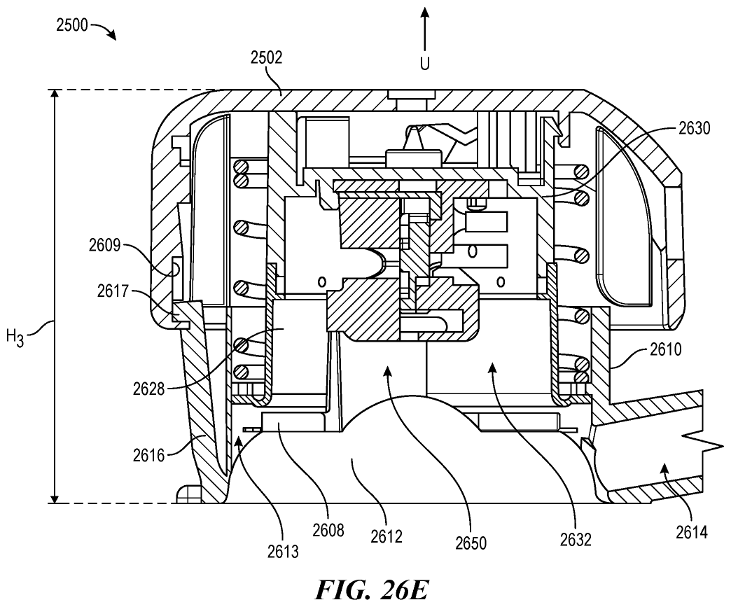

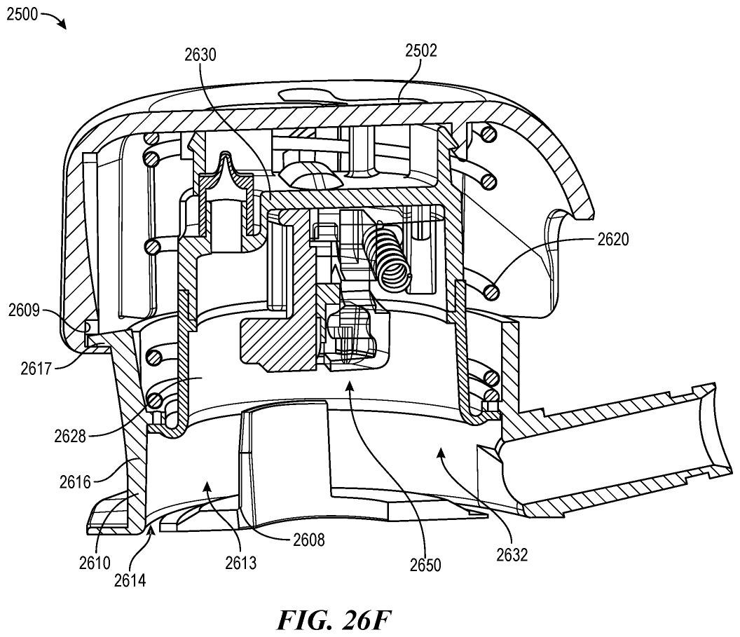

In the illustrated embodiment, the device 2500 includes a housing 2502 (which can also be referred to as an actuator, an actuator-housing, and/or the like), an outflow port 2515, and a lockout mechanism 2503. The lockout mechanism 2503, when positioned as shown in FIG. 25, can inhibit or even prevent movement of the housing 2502 relative to a longitudinal axis L of the device 2500. When the lockout mechanism 2503 is removed, the housing 2502 can be actuated to move along the longitudinal axis L. For example, the housing 2502 can move up (toward the top of the page) and/or down (toward the bottom of the page) and, in some embodiments, at least partially rotate about the longitudinal axis L. More specifically, as described in greater detail below with reference to FIGS. 26A-26F, the housing 2502 can be depressed by a user to move downward to trigger a skin-piercing assembly to incise/cut/puncture skin of a patient, and can move upward thereafter to generate vacuum. Blood can flow from the cut in the skin and out of the outflow port 2515 for collection in a collection reservoir (e.g., a tube; not shown) releasably coupled to the outflow port 2515. Accordingly, the housing 2502 both houses internal components of the device 2500 and also functions as an actuator that can be actuated (e.g., manually actuated) by a user to trigger a blood or other bodily fluid collection process.

FIGS. 26A and 26B are a cross-sectional side view and a perspective cross-sectional side view, respectively, of the device 2500 of FIG. 25 in the first position in accordance with embodiments of the present technology. FIGS. 26C and 26D are a cross-sectional side view and a perspective cross-sectional side view, respectively, of the device 2500 in a second position (e.g., an actuation position, a deployment position) in accordance with embodiments of the present technology. And, FIGS. 26E and 26F are a cross-sectional side view and a perspective cross-sectional side view, respectively, of the bodily fluid collection device 2500 in a third position (e.g., a post-actuated position, a collection position, a vacuum position, an unloaded position) in accordance with embodiments of the present technology.

Referring first to FIGS. 26A and 26B together, in the illustrated embodiment the device 2600 includes a base 2610, a flexible seal 2628 (e.g., a sealing member, a vacuum-generation member), a skin-piercing assembly 2650, a membrane 2612, an island member 2608 (e.g., a floating island), a carrier member 2630, and a biasing member 2620 (e.g., a spring). The base 2610 can be an integral component including a generally-cylindrical wall 2611 defining a corresponding lumen 2613 therethrough that has/terminates at a base opening 2614. The membrane 2612 is coupled to a lower portion of the wall 2611 and extends/spans across the base opening 2614, and the island member 2608 is coupled to (e.g., adhered to, carried by) the membrane 2612. In the illustrated embodiment, the base 2610 further includes an annular ridge 2615 (e.g., a generally cylindrical ridge, an annular member) extending into the lumen 2613. The outflow port 2515 extends from the wall 2611 away from the lumen 2613 and through an opening 2604 in the housing 2502 to outside the housing 2502, where it can be releasably coupled to a collection reservoir (not shown).

In the illustrated embodiment, the carrier member 2630 is coupled to an inner surface/underside 2606 of the housing 2502. In some embodiments, the carrier member 2630 is fixedly coupled to the housing 2502 such that both components move together. For example, the carrier member 2630 can include one or more snap features 2631 configured to engage and secure to a corresponding one or more snap features 2607 extending from the inner surface 2606 of the housing 2502. In other embodiments, the carrier member 2630 can be secured to the housing 2502 in other manners, such as via adhesives, welding, fasteners, and/or the like. In the illustrated embodiment, the carrier member 2630 has an inner surface/underside 2632 that defines a receiving space/volume 2633. The skin-piercing assembly 2650 can be coupled to the inner surface 2632 of the carrier member 2630 via, for example, a snap-fit arrangement, one or more fasteners, welding, adhesives, and/or the like. In some embodiments, the skin-piercing assembly 2650 is fixedly coupled to the carrier member 2630 such that both components move together. Accordingly, in some embodiments the housing 2502, the carrier member 2630, and the skin-piercing assembly 2650 are coupled together to move together.

In the illustrated embodiment, the flexible seal 2628 has a generally annular shape including an outer edge 2640 and an inner edge 2641. The outer edge 2640 can be coupled (e.g., fixedly coupled) to a lower portion/underside of the ridge 2615, and the inner edge 2641 can be coupled to a lower portion 2634 of the carrier member 2630. The carrier member 2630, the flexible seal 2628, and the base 2610 can together define a sealed volume 2635 within the device 2500 with the membrane 2612 and/or when the device 2500 is placed against the skin of a subject. The skin-piercing assembly 2650 is positioned within the sealed volume 2635, and the outflow port 2515 provides an egress for fluid (e.g., blood) to leave the sealed volume for collection in a collection reservoir coupled thereto. In some embodiments, the carrier member 2630 can include a valve 2636 (FIG. 26B) to permit fluid (e.g., air) to exit the sealed volume 2635 and inhibit or even prevent fluid from entering the sealed volume 2635. Accordingly, the valve 2636 can be a one-way valve, such as, for example, a duckbill valve, an umbrella valve, and/or the like.

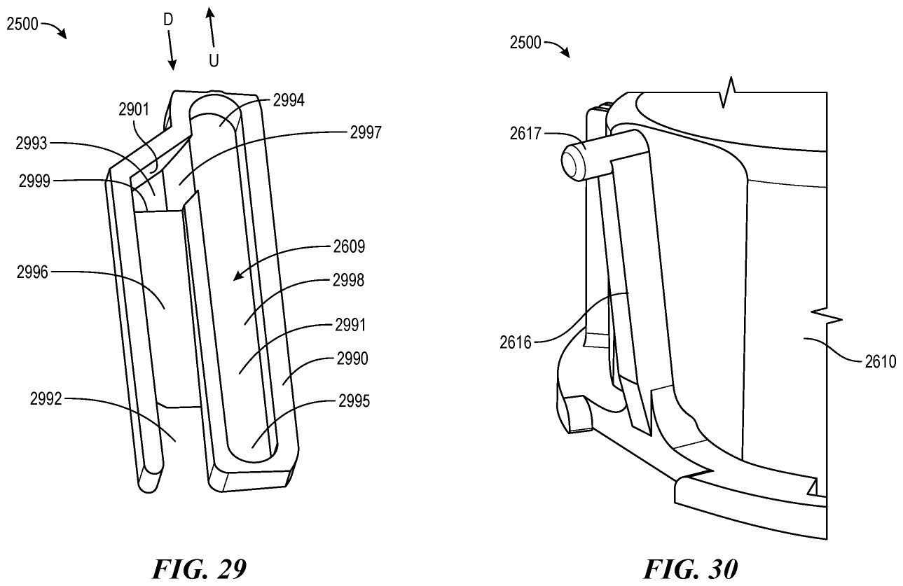

The biasing member 2620 can extend between an upper portion/topside of the ridge 2615 and the inner surface 2606 of the housing 2502. Accordingly, the biasing member 2620 is positioned outside of the sealed volume 2635. The biasing member 2620 can be a coil spring and/or the like and can be configured to bias the housing 2502 away from the base 2610. In the illustrated embodiment, the base 2610 further includes a resilient post 2616 (e.g., arm) having a pin 2617 at a distal end. The post 2616 can be resiliently biased away from an outer surface of the wall 2611 as best seen in FIG. 26A. The pin 2617 is configured to be positioned in a corresponding slot 2609 formed in/defined by the inner surface 2606 of the housing 2502. Further details of the slot 2609 and the pin 2617 are described in detail below with reference to FIGS. 29 and 30. In the first position illustrated in FIGS. 26A and 26B, the engagement of the pin 2617 in the slot 2609 prevents the biasing member 2620 from driving the housing 2502 away from the base 2610. That is, in the first position the biasing member 2620 is in a loaded (e.g., compressed) state storing energy.

Referring to FIG. 26B, the housing 2502 further includes a lower rim 2660, and the base 2610 includes a lower flange 2618 extending outward away from the wall 2611. In the first position, the lower rim 2660 can be spaced apart (e.g., spaced upwardly away) from the lower flange 2618 by a gap G. The lockout mechanism 2503 can be positioned in the gap G as shown in FIG. 26A to substantially prevent movement of the housing 2502 toward the base 2610 and, thereby, to prevent inadvertent actuation of the device 2500. More specifically, the lockout mechanism 2503 can include a pull portion 2661 and a blocking portion 2662. The blocking portion 2662 can be configured (e.g., shaped, sized) to fit into the gap G between the lower rim 2660 and the lower flange 2618, thereby filling the space therebetween. The pull portion 2661 can be gripped by a user and pulled or otherwise actuated to remove the blocking portion 2662 from the gap to permit relative movement between the housing 2502 and the base 2610, thereby permitting actuation of the device 2500.

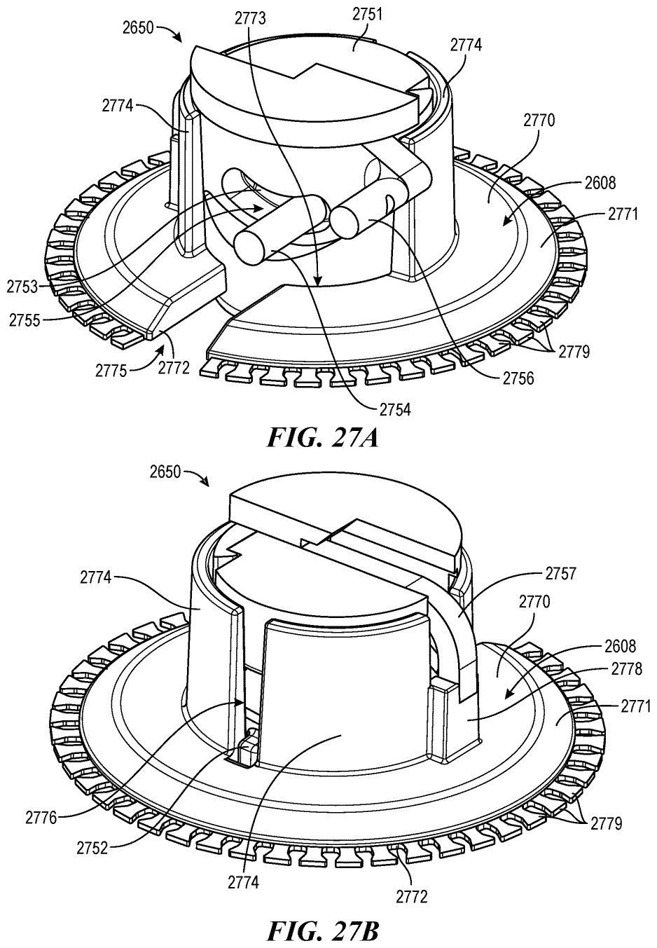

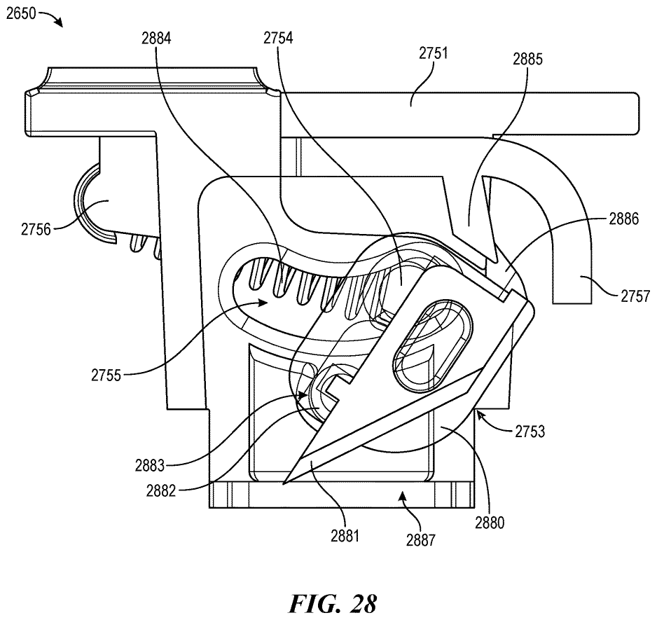

Referring to FIGS. 26A-26D together, once the lockout mechanism 2503 is removed, to move the device 2500 from the first position (FIGS. 26A and 26B) to the second position (FIGS. 26C and 26D), a user can depress the housing 2502 toward the base 2610 to drive the carrier member 2630 and the skin-piercing assembly 2650 downward through the lumen 2613 in the base 2610 toward the base opening 2614. As described in detail above and below with reference to FIGS. 27A-28, the skin-piercing assembly 2650 can engage a portion of the island member 2608, which triggers the skin-piercing assembly 2650 to deploy a blade or other cutting member through the base opening 2614 and into the skin of a subject. And, as described in detail below with reference to FIGS. 29 and 30, movement of the housing 2502 can drive the pin 2617 through the slot 2619 to, for example, permit subsequent upward movement of the housing 2502 relative to the base 2610 to generate vacuum pressure in the sealed volume 2635. The downward movement of the carrier member 2630 and the flexible seal 2628 coupled thereto can decrease a volume of the sealed volume 2635, and the valve 2636 can vent air from the sealed volume 2635.

Referring to 26C-26F together, to move the device 2500 from the second position (FIGS. 26C and 26D) to the third position (FIGS. 26E and 26F), the user can release the housing 2502 to permit the biasing member 2620 to drive the housing 2502 upward and the carrier member 2630 and the skin-piercing assembly 2650 upward through the lumen 2613 in the base 2610 away from the base opening 2614. The upward movement of the carrier member 2630 and the flexible seal 2628 coupled thereto can increase a volume of the sealed volume 2635 to generate vacuum pressure within the sealed volume 2635. In some embodiments, the vacuum pressure can be between about −30 kilopascals (kPA) and about −60 kPA, between about −38 kPA and about −49 kPA, and/or the like. As best seen in FIG. 26E, and as described in detail above, the vacuum pressure can pull at least a portion of the membrane 2612 (and skin beneath the membrane 2612) into the sealed volume 2635 in the lumen 2613 in the base 2610. The island member 2608 can shape the membrane 2612 to have a desired shape, curvature, and/or the like to facilitate efficient bodily fluid (e.g., blood) collection. And, as described in detail below with reference to FIGS. 29 and 30, movement of the housing 2502 from the second position to the third position can drive the pin 2617 through the slot 2619 to a final position shown in FIGS. 26E and 26F in which the pin 2617 inhibits further upward movement of the housing 2502 and retains the housing 2502 coupled to the base 2610. In some embodiments, the arrangement of the pin 2617 and the slot 2619 allows the housing 2502 to be depressed from the third position to the second position and subsequently released to move back from the second position to the third position to allow for additional vacuum generation within the sealed volume 2635.

Referring to FIGS. 26A, 26C, and 26E, the device 2500 can have a first height H₁ in the first position (FIG. 26A), a second height H₂ in the second position (FIG. 26C), and a third height H₃ in the third position (FIG. 26E). The second height H₂ can be less than the first height H₁, and the third height H₃ can be greater than the first height H₁ and the second height H₃. For example, the first height H₁ can be about 22 millimeters, the second height H₂ can be about 20 millimeters, and the third height H₃ can be about 34.5 millimeters. Accordingly, the device 2500 can have a smaller height and thus overall profile in the pre-actuated first position than in the actuated third position. In some aspects of the present technology, the relatively small/compact profile of the device 2500 in the pre-actuated first position can facilitate easier transportation, storage, shipping, and/or the like of the device 2500.

FIGS. 27A-32 illustrate various features of the device 2500 of FIGS. 25-26F with, for example, additional detail and specificity. FIGS. 27A and 27B, for example, are a front perspective view and a back perspective view, respectively, of the skin-piercing assembly 2650 and the island member 2608 in accordance with embodiments of the present technology. Referring to FIGS. 27A and 27B together, the island member 2608 comprises a generally flat, annular base portion 2770 having an upper surface 2771 and a lower surface 2772, a central opening 2773 extending through the base portion 2770, one or more projections 2774 extending upward from the upper surface 2771 at least partially circumferentially around the central opening 2773, and a cutout 2275 (FIG. 27A) through the opening. The lower surface 2772 is configured to be coupled to (e.g., adhered to, carried by) the membrane 2612 (FIGS. 26A-26F). The cutout 2775 can define a flow path for blood or other bodily fluid to flow across the membrane 2612 from the central opening 2773 to the outflow port 2515 (FIGS. 25-26F).

In the illustrated embodiment, the skin-piercing assembly 2650 includes a housing 2751 constrained to move between the projections 2774. Referring to FIG. 27B, in some embodiments the housing 2751 can include one or more key portions (e.g., tabs, ribs) 2752 constrained to travel substantially vertically within a corresponding slot 2776 formed between an adjacent pair of the projections 2774, thereby substantially inhibiting rotation of the housing 2751 relative to the island member 2608. Referring to FIG. 27A, the housing 2751 can house a drive assembly 2753 (FIG. 27A) having a blade as described in greater detail below with reference to FIG. 28. The drive assembly 2753 can be coupled to (e.g., integrally formed with) a first pin 2754 constrained to move within a slot 2755 in the housing 2751. The skin-piercing assembly 2650 can further include a post 2756 coupled to (e.g., integrally formed with) the housing 2751. A biasing member (e.g., an extension spring) can be coupled between the first pin 2754 and the post 2756 as described in greater detail below with reference to FIG. 28 to drive the first pin 2754 along the slot 2755 to thereby drive the drive assembly 2753 within the housing 2751. Referring to FIG. 27B, the skin-piercing assembly 2650 can further include a deflectable arm 2757, and the island member 2608 can include a deployment post 2778 extending upward from the upper surface 2771. Downward movement of the skin-piercing assembly 2650 relative to the island member 2608 can engage the arm 2757 with the deployment post 2778 to deflect the arm 2757 outward away from the housing 2751 to allow the biasing member to drive the first pin 2754 along the slot 2755 as described in greater detail below with reference to FIG. 28.

Referring again to FIGS. 27A and 27B, the island member 2608 can further include a plurality of tabs 2779 (e.g., fingers) around an outer circumference thereof. The tabs 2779 can be configured (sized, dimensioned) to flex when a vacuum is drawn, and the membrane 2612 (FIGS. 26A-26F) is drawn into the device 2500. In some aspects of the present technology, the tabs 2779 provide for a relatively softer outer edge of the island member 2608 that can, for example, inhibit or even prevent bruising of skin positioned at and/or proximate the outer edge of the island member 2608.

FIG. 28 is a partially-transparent side view of the skin-piercing assembly 2650 in accordance with embodiments of the present technology. In the illustrated embodiment, the housing 2751 houses the drive assembly 2753, which includes a drive member 2880 and a blade 2881 coupled to the drive member 2880. A portion of the housing 2751 is removed, and the drive member 2880 is shown as partially transparent in FIG. 28 for clarity. The first pin 2754 extends from the drive member 2880 through the slot 2755 (e.g., a first slot 2755). The drive member 2880 can further include a second pin 2882 extending into a second slot 2883 in the housing 2751. In some embodiments, the second slot 2883 constrains the second pin 2882 to move generally vertically, while the first slot 2755 constrains the first pin 2754 to move generally horizontally and partially vertically (e.g., in a downward arc). A biasing member 2884, such as an extension spring, extends between the post 2756 and the first pin 2754.

In the illustrated embodiment, the skin-piercing assembly 2650 is in a pre-actuated or pre-deployed position corresponding to the position of the device 2500 in FIGS. 25-26B. In this position, a holding projection 2885 of the deflectable arm 2757 engages a retaining tab 2886 on the drive member 2880 to inhibit the biasing member 2884 from driving/moving the first pin 2754 along the first slot 2755. With additional reference to FIGS. 27A and 27B, when the skin-piercing assembly 2650 is driven downward relative to the island member 2608, the deflectable arm 2757 is deflected outward and upward such that the holding projection 2885 disengages the retaining tab 2886 of the drive member 2880. Then, the biasing member 2884 can pull the first pin 2754 along the slot 2755 to partially rotate the drive member 2880 such that the blade 2881 moves downward toward and through an opening 2887 in the housing 2751 to incise the skin of a patient. After the first pin 2754 moves along all or substantially all of the first slot 2755 (e.g., in the right-to-left direction), movement of the drive assembly 2753 ceases, and the blade 2881 is again positioned entirely within the housing 2751 and cannot be re-actuated.

FIGS. 29 and 30 are perspective views of the slot 2609 in the housing 2502, and the post 2616 and pin 2617 of the base 2610, respectively, of the device 2500 of FIGS. 26A-26F in accordance with embodiments of the present technology. Referring to FIGS. 29 and 30, the slot 2609 can include one or more walls 2990 defining a track 2991, and the pin 2617 can be constrained to move along the track 2991 by the wall 2990. Referring to FIG. 29, the track 2991 defines/includes an entrance 2992, a first pin position 2993, a second pin position 2994, and a third pin position 2995. The track 2991 further includes a first track portion 2996 between the entrance 2992 and the first pin position 2993, a second track portion 2997 between the first pin position 2993 and the second pin position 2994, and a third track portion 2998 between the second pin position 2994 and the third pin position 2995.

Referring to FIGS. 26A-26F, 29, and 30 together, during assembly of the device 2500 the pin 2617 can be inserted into the entrance 2992 and slid along the first track portion 2996 to the first pin position 2993. The first pin position 2993 corresponds to the first position of the device shown in FIGS. 26A-26F. In some embodiments, the first track portion 2996 includes a step 2999 (e.g., a wall, a ledge) positioned proximate to the first pin position 2993. When the pin 2617 slides past the step 2999 toward the first pin position 2993, the resilient post 2616 can drive the pin 2617 downward past the step 2999 such that the pin 2617 cannot return from the first pin position 2993 toward and/or out of the entrance 2992. That is, for example, the step 2999 is configured to constrain the pin 2617 to be able to move only from the first pin position 2993 toward the second pin position 2994. Moreover, in the first position of the device 2500, the engagement of the pin 2617 with the step 2999 prevents the biasing member 2620 from driving the housing 2502 away from the base 2610.

When the housing 2502 is depressed in a direction D (FIGS. 26C and 29) to move the device from the first position to the second position, the pin 2617 contacts an angled wall portion 2901 of the wall 2990 such that the housing 2502 is rotated partially about the longitudinal axis L (FIG. 25) and the pin 2617 is driven along the second track portion 2997 to the second pin position 2994. The second pin position 2994 corresponds to the second position of the device 2500 (FIGS. 26C and 26D) in which the skin-piercing assembly 2650 deploys/fires the blade 2881 to cut the skin of a patient. In some embodiments, the second track portion 2997 is angled upward such that movement of the pin 2617 from the first pin position 2993 to the second pin position 2994 deflects the post 2616 radially inward. In some embodiments, the angled second track portion 2997 can inhibit or even prevent accidental/incidental movement of the pin 2617 from the first pin position 2993 to the second pin position 2994. In some embodiments, the pin 2617 engages the wall 2990 in the second pin position 2994 to inhibit further depression of the housing 2502 in the direction D. Accordingly, a vertical position of the second pin position 2994 relative to the first pin position 2993 can dictate a maximum stroke of the housing 2502.

Once the pin 2617 is in the second position and a force on the housing 2502 is released, the biasing member 2620 can drive the housing 2502 upward in a direction U (FIGS. 26E and 29). The movement of the housing 2502 drives the pin 2617 along the third track portion 2998 to the third pin position 2995. The third pin position 2995 corresponds to the third position of the device 2500 (FIGS. 26E and 26F) in which the skin-piercing assembly 2650 draws a vacuum in the sealed volume 2635 for collecting bodily fluid (e.g., blood). In some embodiments, the pin 2617 engages the wall 2990 in the third pin position 2995 to inhibit further withdrawal of the housing 2502 in the direction U. In some embodiments, the housing 2502 can be depressed/released to generate additional vacuum within the sealed volume 2635. On each such stroke, the pin 2617 can move from the third pin position 2995 back to the second pin position 2994 and then back from the second pin position 2994 to the third pin position 2995. In some aspects of the present technology, the pin 2617 is constrained to move only along the third track portion 2998 between the second pin position 2994 and the third pin position 2995 after the pin 2617 reaches the second pin position 2994.

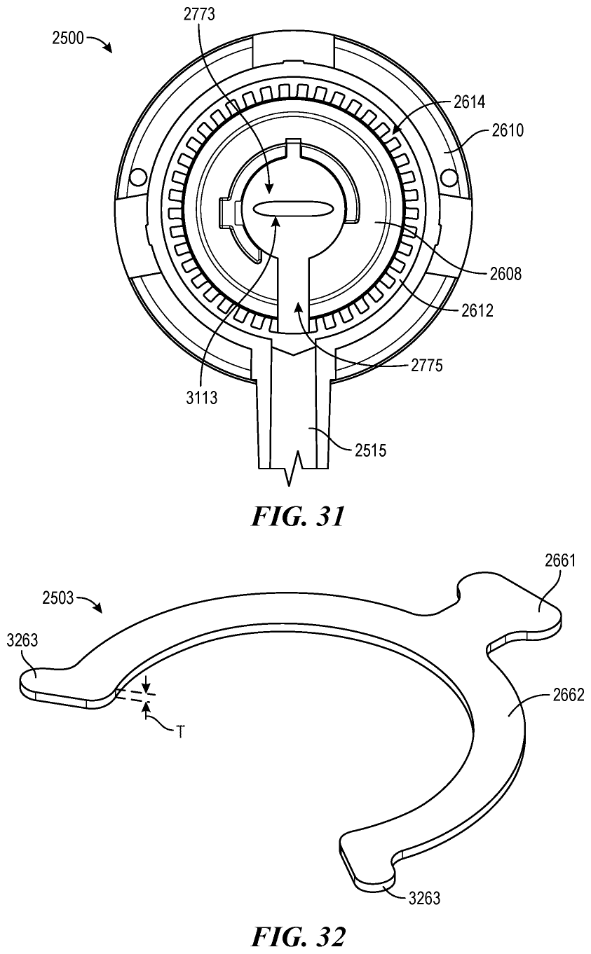

FIG. 31 is a top view of the base 2610, the membrane 2612, and the island member 2608 of the device 2500 of FIGS. 25-26F in accordance with embodiments of the present technology. In the illustrated embodiment, the membrane 2612 extends across the base opening 2614, and the island member 2608 is coupled to (e.g., adhered to, carried by) the membrane 2612. The membrane 2612 can carry/move/float the island member 2608 during actuation of the device 2500 when, for example, vacuum is generated within the device 2500. The cutout 2775 in the island member 2608 is aligned with the outflow port 2515 to define a flow path for blood or other bodily fluid to flow across the membrane 2612 from the central opening 2773 in the island member 2608 to the outflow port 2515. In some embodiments, the membrane 2612 includes a pre-formed slit 3113 formed therein and through which the blade 2881 (FIG. 28) can travel to puncture the skin of a subject. In some embodiments, the slit 3113 is elongate and extends along a longitudinal axis that is generally perpendicular to the outflow port 2515 and the cutout 2775.

FIG. 32 is a perspective of the lockout mechanism 2503 of the device 2500 of FIGS. 25-26F in accordance with embodiments of the present technology. The lockout mechanism 2503 includes the pull portion 2661 and the blocking portion 2662. The blocking portion 2602 can have a partial ring shape, including end tabs 3263. Referring to FIGS. 26B and 32 together, in some embodiments the lockout mechanism 2503 can have a thickness T that is the same or slightly less than a height of the gap between the lower rim 2660 and the lower flange 2618. For example, the thickness T can be about 1.0 millimeter, and the height of the gap can be about 1.1 millimeters. In some embodiments, the pull portion 2661 and/or one or both of the end tabs 3263 can be grasped by a user and used to pull the lockout mechanism 2503 from the gap and/or to rotate the lockout mechanism 2503 to either side to permit actuation of the device 2500.

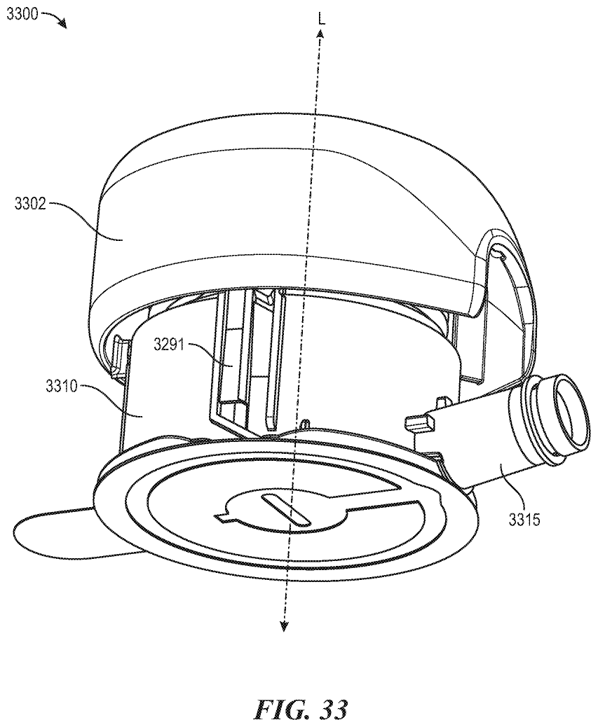

FIG. 33 is a partially exploded perspective view of a bodily fluid collection device 3300 in accordance with additional embodiments of the present technology. The device 3300 can include some features that are at least generally similar in structure and function, or identical in structure and function, to the corresponding features of any of the bodily fluid collection devices described in detail above with reference to FIGS. 1A-32, and can operate in a generally similar or identical manner to any of those devices.

In the illustrated embodiment, the device 3300 includes a housing 3302 (which can also be referred to as an actuator, an actuator-housing, and/or the like), a base 3310, and an outflow port 3315. In contrast to the device 2500 described in greater detail above with reference to FIGS. 25-32, a track 3291 of the device 3200 is positioned on the base 3310, and a pin 3317 (obscured) is positioned on the housing 3302 and/or integrally formed with the housing 3302. The track 3291 and the pin 3217 can include some features that are at least generally similar in structure and function, or identical in structure and function, to the corresponding features of the track 2991 and the pin 2617 described in greater detail above, and can operate in a generally similar or identical manner. For example, the track 3291 can have an identical shape, size, and/or configuration to the track 2991, and the pin 3317 can slide through the track 3291 to permit controlled actuation and retraction of the device 3300 in a like manner to the device 2500 described in detail above.

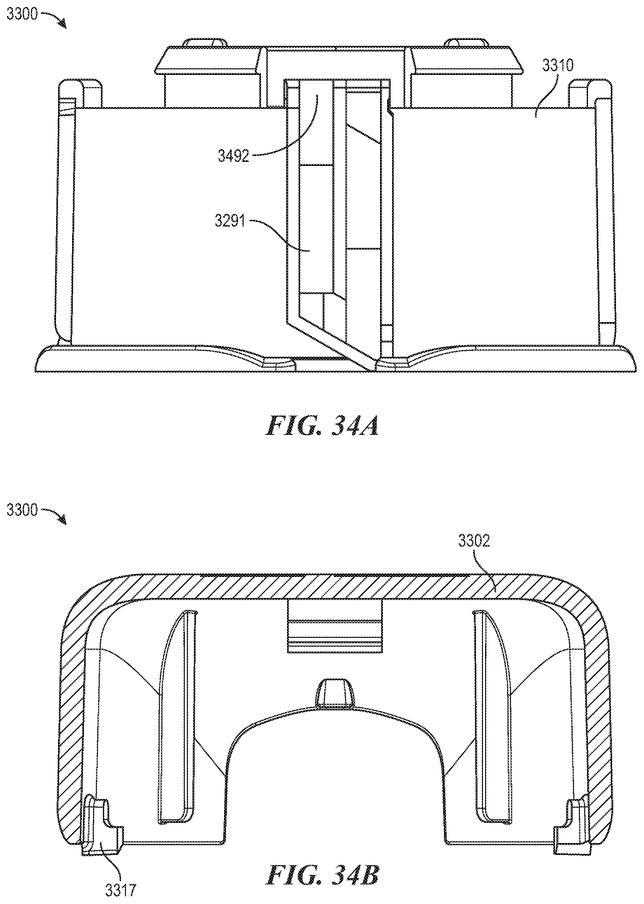

FIG. 34A is a side view of the base 3310 of the bodily fluid collection device 3300 of FIG. 33 in accordance with embodiments of the present technology. FIG. 34B is a side view of the housing 3302 of the bodily fluid collection device 3300 of FIG. 33 in accordance with embodiments of the present technology. Referring to FIGS. 34A and 34B together, as described above, the track 3391 is positioned on the base 3310, and the pin 3317 is positioned on the housing 3302. Because the track 3391 is positioned on the stationary base 3310, an entrance 3492 of the track 3391 can be positioned at/near a top (e.g., an upper portion) of the base 3310 to receive the pin 3317. More specifically, because the pin 3317 is in a fixed position near a bottom of the housing 3302, the pin 3317 can enter the track 3391 at the entrance 3492 positioned at the top of the base 3310 during assembly of the device 3330. During operation of the device 3300, the pin 3317 can pass through the track 3391 as described in greater detail above with reference to FIG. 29.

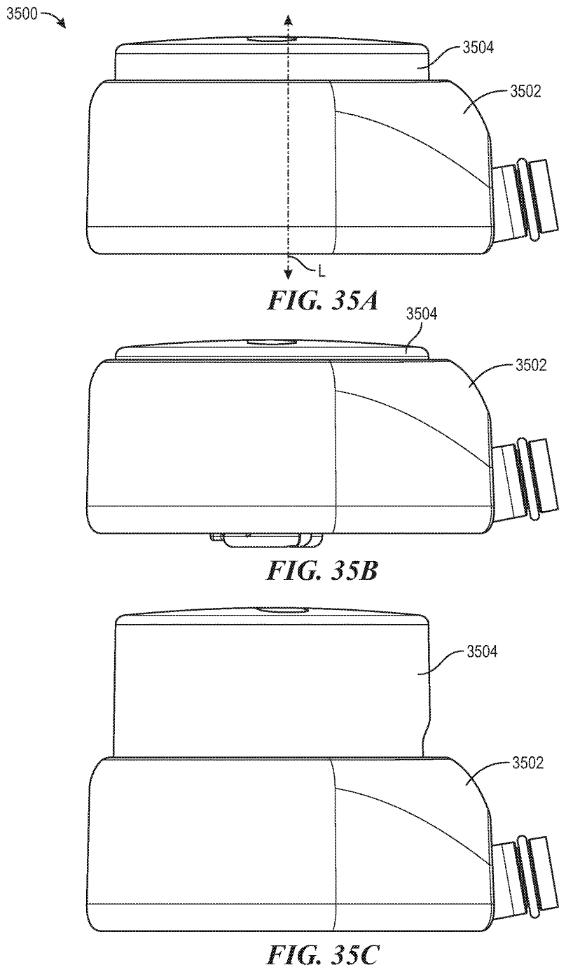

FIGS. 35A-35C are perspective views of a bodily fluid collection device 3500 in a first position, a second position, and a third position, respectively, in accordance with embodiments of the present technology. The device 3500 can include some features that are at least generally similar in structure and function, or identical in structure and function, to the corresponding features of any of the bodily fluid collection devices described in detail above with reference to FIGS. 1A-34B, and can operate in a generally similar or identical manner to any of those devices.

In the illustrated embodiments, the device 3500 includes a housing 3502 and an actuator 3504 operably coupled to the housing 3502. The actuator 3504 can include some features that are at least generally similar in structure and function, or identical in structure and function, to the corresponding features of any of the actuators described in detail above with reference to FIGS. 1A-34B, and can operate in a generally similar or identical manner to any of those actuators. For example, rather than depressing the entire housing 3502 to actuate the device 3500, the user can actuate the actuator 3504 extending through the housing 3502, and the position of the housing 3502 can remain fixed relative to the base 3510 (obscured). That is, for example, compared to the embodiments in FIGS. 25-34B, the device 3500 includes a separate actuator and housing rather than a single integrally formed housing-actuator. Other internal components of the device 3500 (e.g., seal member, island member, etc.) can be similar or identical in structure and/or function.

Referring to FIG. 35A, the first position is an undeployed position (e.g., the actuator 3504 has not been pressed). Referring to FIG. 35B, the second position is an actuation or deployment position where the actuator 3504 is pressed (e.g., actuated). Referring to FIG. 35C, the third position is a post-actuated position, a collection position, a vacuum position, and/or an unloaded position. In the third position, the actuator 3504 extends upward from the housing 3502 farther than the actuator 3504 extended upward in the first position. In some aspects of the present technology, this can indicate to the user that the device 3500 has been used/deployed.

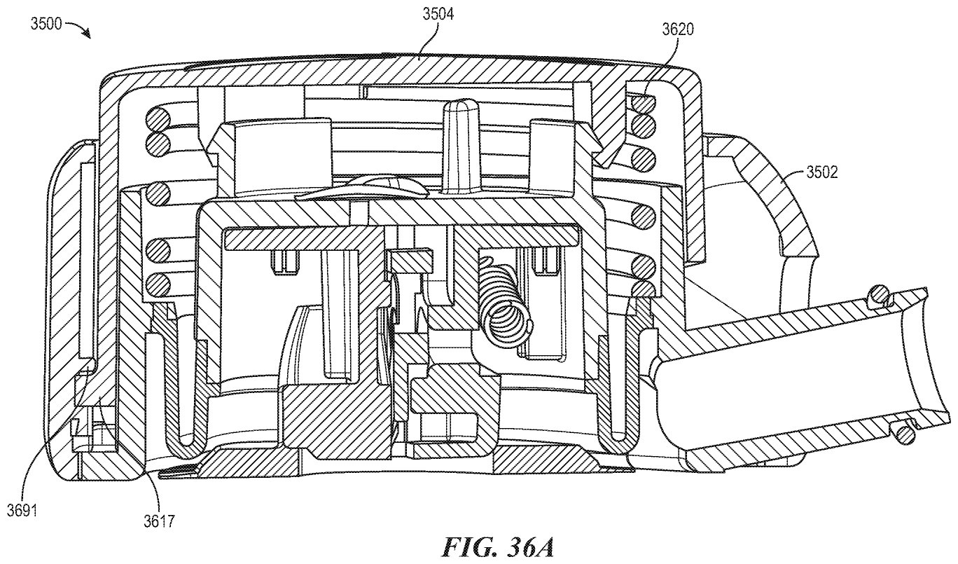

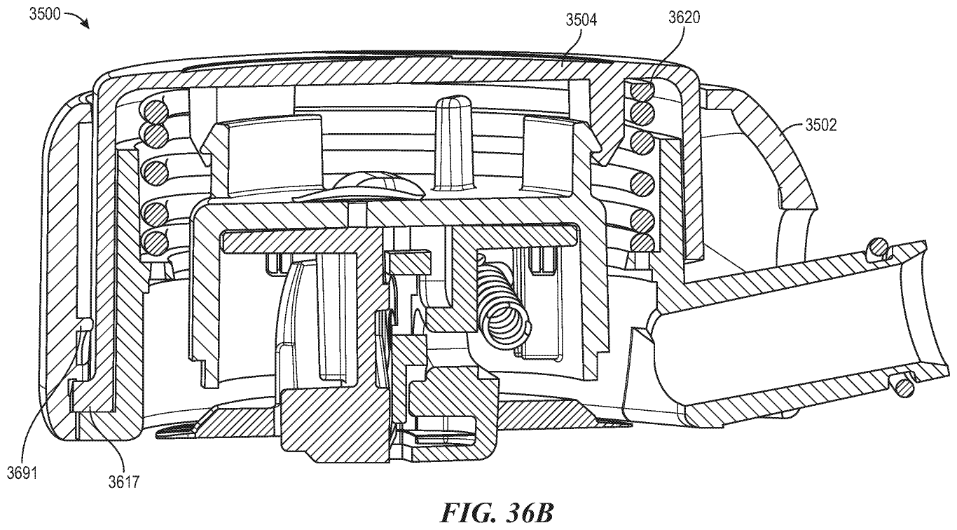

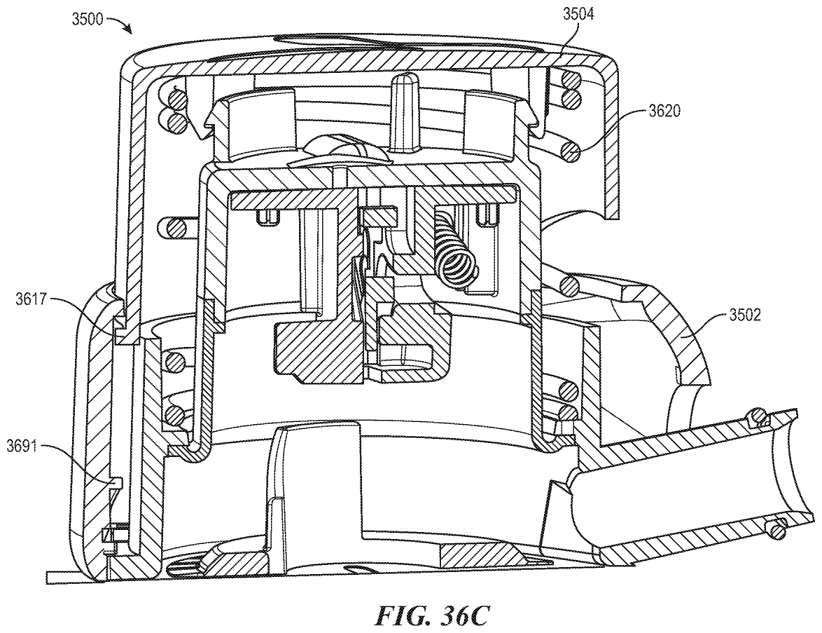

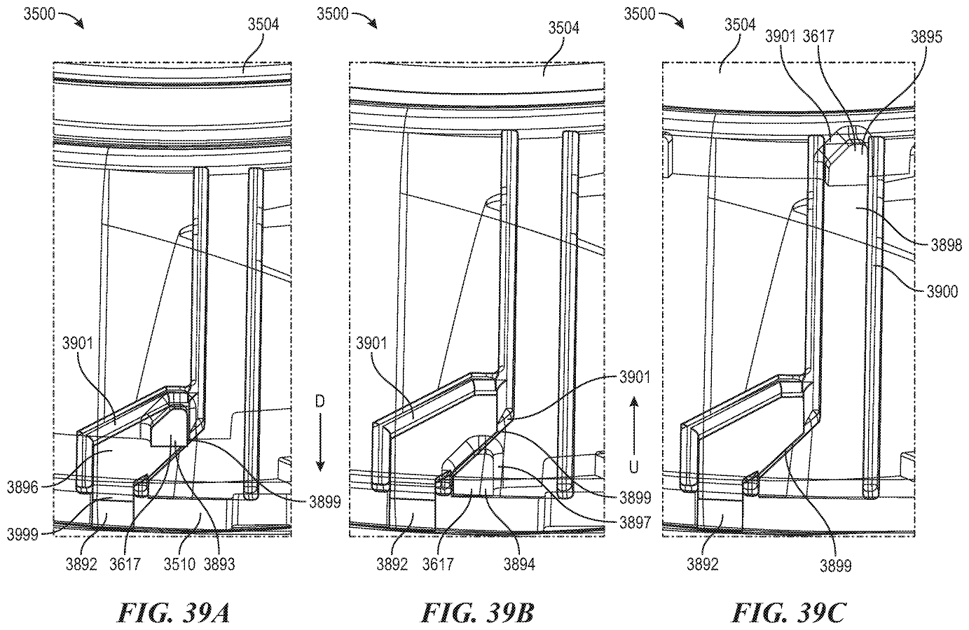

FIGS. 36A-36C are cross-sectional perspective views of the bodily fluid collection device 3500 of FIGS. 35A-35C in the first position, the second position, and the third position, respectively, in accordance with embodiments of the present technology. Referring to FIGS. 36A-36C together, the actuator 3504 of the device 3500 includes a pin 3617, and the housing 3502 includes a track 3691. During operation of the device 3500 (e.g., movement from the first position to the third position), the pin 3617 passes through the track 3691, as described in greater detail below with reference to FIG. 39A-39C, restricting movement of the actuator 3504 to inhibit re-actuation of the device 3500. Further, the actuator 3504 is biased upward by a biasing member 3620.

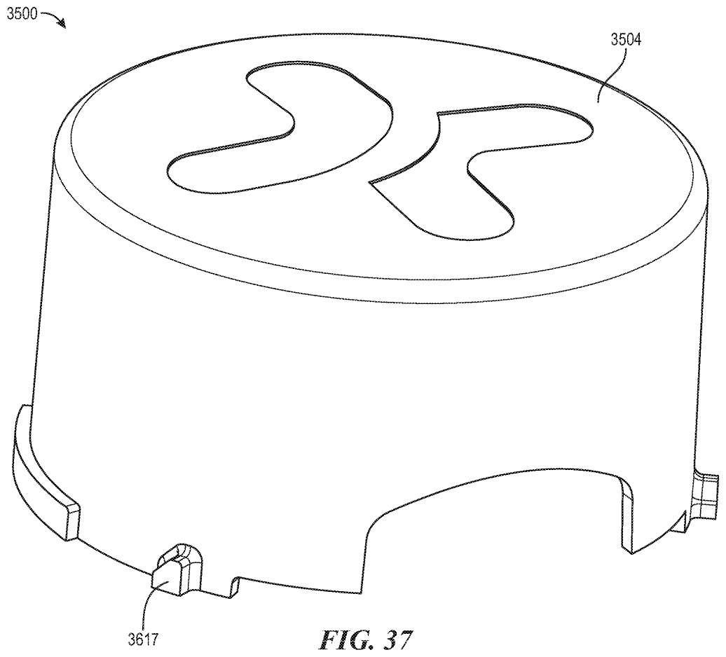

FIG. 37 is a perspective view of the actuator 3504 of the bodily fluid collection device 3500 of FIGS. 35A-36B in accordance with embodiments of the present technology. In the illustrated embodiment, the actuator 3504 includes the pin 3617 fixedly positioned on the actuator 3504 and/or integrally formed with the actuator 3504. In some embodiments, the actuator 3504 can include one or more pins 3617 that complement one or more tracks 3691 (FIG. 36A) of the housing 3502 (FIG. 36A). The pin 3617 can be positioned toward and/or at a bottom (e.g., a bottom end portion) of the actuator 3504.

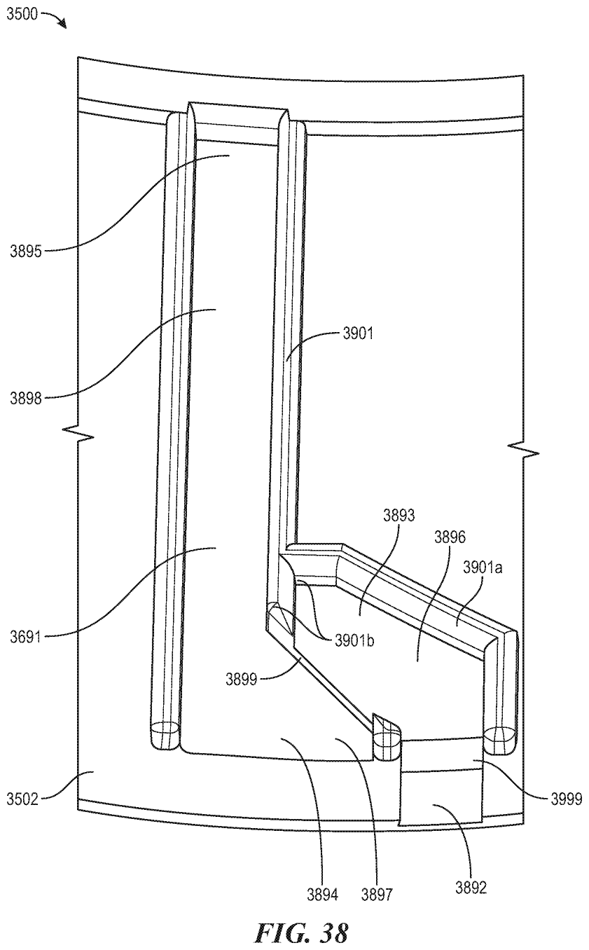

FIG. 38 is a perspective view of a portion of the housing 3502 of the bodily fluid collection device 3500 of FIGS. 35A-36B, including the track 3691 in accordance with embodiments of the present technology. In the illustrated embodiment, one or more walls 3901 define the track 3691, and the pin 3617 can be constrained to move along the track 3691 by the wall(s) 3901. The track 3691 can define/include an entrance 3892, a first pin position 3893, a second pin position 3894, and a third pin position 3895. The track 3691 further includes a first track portion 3896 between the entrance 3892 and the first pin position 3893, a second track portion 3897 between the first pin position 3893 and the second pin position 3894, and a third track portion 3898 between the second pin position 3894 and the third pin position 3895. FIGS. 39A-39C are perspective, partially transparent views of the bodily fluid collection device 3500 of FIGS. 35A-35C in the first position, the second position, and the third position, respectively, in accordance with embodiments of the present technology.

Referring to FIGS. 36A-36C and 38-39C together, during assembly of the device 3500, the pin 3617 can be inserted into the entrance 3892 and slid along the first track portion 3896 to the first pin position 3893. The first pin position 3893 corresponds to the first position of the device 3500 shown in FIGS. 36A and 39A. In some embodiments, the first track portion 3896 includes a ramp 3999 positioned proximate to the entrance 3892. When the pin 3617 slides past/along the ramp 3999 toward the first pin position 3893 (e.g., during assembly of the device 3500), the pin 3617 (e.g., formed of a resilient plastic) can be biased outward away from a longitudinal vertical axis of the device 3500 and store potential energy. An angled portion 3901a of the wall 3901 can guide the pin 3617 to the first pin position 3893. In the first position of the device 3500, the engagement of the pin 3617 with a notched portion 3901b of the wall 3901 prevents the biasing member 3620 (FIG. 36A) from driving the actuator 3504 away from the base 3510. Moreover, in the illustrated embodiment the first pin position 3893 is laterally rotated relative to the entrance 3892 such that the pin 3617 cannot return from the first pin position 3893 toward and/or out of the entrance 3892 and is instead constrained only to move further through the track 3691.

The second pin position 3894 corresponds to the second position of the device 3500 (FIGS. 36B and 39B) in which a skin-piercing assembly deploys/fires a blade to cut the skin of a patient. In some embodiments, the second track portion 3897 includes a step 3899 (e.g., a wall, a ledge) positioned proximate to the first pin position 3893. When the actuator 3504 (FIGS. 35A-36B) is pressed downward, the pin 3617 slides past the step 3899 toward the second pin position 3894, and the stored potential energy in the pin 3617 (e.g., as a result of the outward biasing from the ramp 3999) causes the pin 3617 to move inward toward the longitudinal axis L (FIG. 35A) below the step 3899 such that the pin 3617 cannot return from the second pin position 3894 toward the first pin position 3893. That is, for example, the step 3899 is configured to constrain the pin 3617 to be able to move only from the first pin position 3893 toward the second pin position 3894.

Once the pin 3617 is in the second position and a force on the actuator 3504 is released, the biasing member 3620 (FIG. 36A) can drive the actuator 3504 upward in a direction U. When the actuator 3504 is released in the direction U, the pin 3617 moves upward and contacts the step 3899, which is angled, such that the actuator 3504 is rotated partially about the longitudinal axis L (FIG. 35A) and the pin 3617 is driven along the third track portion 3898 to the third pin position 3895. The third pin position 3895 corresponds to the third position of the device 3500 (FIGS. 36C and 39C) in which the skin-piercing assembly draws a vacuum for collecting bodily fluid (e.g., blood). In some embodiments, the pin 3617 engages the wall 3901 in the third pin position 3895 to inhibit further withdrawal of the actuator 3504 in the direction U. In some aspects of the present technology, rotating the actuator 3504 between the second pin position 3894 and the third pin position 3895 provides a better user experience. Namely, the user only engages with the actuator 3504 to push the actuator 3504 downward from the first position (FIG. 36A) to the second position (FIG. 36B). During that user actuation, the pin 3617 moves linearly downward from the first pin position 3893 to the second pin position 3894 without rotation of the actuator 3504. Only after the user releases the actuator 3504 to permit the actuator 3504 to move from the second position (FIG. 36B) to the third position (FIG. 36C) does the actuator 3504 slightly rotate (via contact of the pin 3617 with the angled step 3899) to permit the pin 3617 to move along the third track portion 3898. Accordingly, the actuator 3504 does not rotate when the user engages the actuator 3504 to operate the device 3500.

In some embodiments, the actuator 3504 can be depressed/released to generate additional vacuum. On each such stroke, the pin 3617 can move from the third pin position 3895 back to the second pin position 3894 and then back from the second pin position 3894 to the third pin position 3895 after, for example, the user releases the actuator 3504. In some aspects of the present technology, the pin 3617 is constrained to move only along the third track portion 3898 between the second pin position 3894 and the third pin position 3895 after the pin 3617 reaches the second pin position 3894. In other embodiments, the actuator 3504 can include a lockout feature that inhibits regeneration of vacuum. For example, the third track portion 3898 can include a ledge or wall near the third pin position 3895 that inhibits or even prevents movement of the pin 3617 from the third pin position 3895 toward the second pin position 3894.

### III. EXAMPLES

The present technology is illustrated, for example, according to various aspects described below. Various examples of aspects of the present technology are described as numbered examples (1, 2, 3, etc.) for convenience. These are provided as examples and do not limit the present technology. It is noted that any of the dependent examples can be combined in any suitable manner and placed into a respective independent example. The other examples can be presented in a similar manner.
1. A device for withdrawing bodily fluid from a subject, the device comprising:
a housing having a surface configured to be positioned against skin of the subject, the housing having an opening extending through the surface;
a flexible membrane stretched across the opening;
a floating island carried by the flexible membrane and adhesively coupled to the flexible membrane;
a skin-piercing assembly positioned at least partially within the housing and having a blade configured to pierce the skin of the subject; and
an actuator operably coupled to the skin-piercing assembly, wherein the actuator is movable relative to the housing to deploy the skin-piercing assembly through the opening and into the skin of the subject, and
   wherein—
      movement of the actuator away from the opening draws a vacuum within at least a portion of the housing, and
      the skin of a patient is configured to be drawn at least partially through the opening in the housing and assume a two-tiered shape therein when the vacuum is drawn within the housing.
2. The device of example 1 wherein all or substantially all of the surface of the housing comprises the opening.
3. The device of example 1 wherein the opening has a vacuum diameter of about 30 mm.
4. The device of any of examples 1-3 wherein the floating island is a half-moon shape.
5. The device of example 1-4 wherein the housing further comprises a one-way valve configured to allow gas to flow out of the housing and inhibit gas from flowing into the housing.
6. The device of any of examples 1-5 wherein the flexible membrane defines a precut hole aligned with the blade of the skin-piercing assembly.
7. The device of any of examples 1-6 wherein the floating island comprises walls configured to direct bodily fluid flow towards an outflow port of the housing.
8. The device of any of examples 1-7 wherein the housing further comprises a biasing member configured to bias the actuator and the skin-piercing assembly away from the opening.
9. The device of any of examples 1-8 wherein an inner surface of the housing is covered by a film bridge.
10. The device of any of examples 1-9 wherein the housing includes walls configured to direct bodily fluid flow towards an outflow port.
11. The device of any of examples 1-10 wherein the floating island includes features configured to deploy the blade of the skin-piercing assembly.
12. a Device Comprising:
a housing having a surface configured to be positioned against skin of a subject, the housing having an opening extending through the surface;
a flexible membrane stretched across the opening;
a floating island carried by the flexible membrane and adhesively coupled to the flexible membrane;
a skin-piercing assembly positioned at least partially within the housing and having a blade configured to pierce the skin of the subject when deployed; and
   wherein—
      movement of the skin-piercing assembly away from the opening draws a vacuum within at least a portion of the housing, and
      the skin of the subject is drawn at least partially through the opening in the housing and assumes a two-tiered shape therein when subject to the vacuum.
13. The device of example 12 wherein the skin-piercing assembly includes one or more blades configured to make one or more cuts on the skin.
14. The device of either example 12 or 13 wherein the floating island is centered on the flexible membrane.
15. The device of any of examples 12-14 wherein the floating island includes flanges configured to direct fluid across the flexible membrane and towards an outflow port.
16. The device of any of examples 12-15 wherein a first tier of the two-tiered shape is larger than a second tier of the two-tiered shape.
17. A method of collecting blood from a patient, comprising:
placing a bodily fluid collection device on skin of the patient;
pressing an actuator of the bodily fluid collection device to deploy a blade to cut the patient's skin;
releasing the actuator to create a vacuum within the bodily fluid collection device to increase bodily fluid flow from a wound in the patient's skin;
collecting a desired sample in a collection reservoir; and
removing the bodily fluid collection device from the skin.
18. The method of example 17 wherein pressing the actuator further comprises forcing air out of the bodily fluid collection device.
19. The method of either example 17 or 18 wherein releasing the actuator causes a biasing member of the bodily fluid collection device to bias the actuator away from the skin.
20. The method of any of examples 17-19 further comprising separating the collection reservoir from the bodily fluid collection device prior to removing the bodily fluid collection device from the skin.

### IV. CONCLUSION

All numeric values are herein assumed to be modified by the term about whether or not explicitly indicated. The term about, in the context of numeric values, generally refers to a range of numbers that one of skill in the art would consider equivalent to the recited value (e.g., having the same function and/or result). For example, the term about can refer to the stated value plus or minus ten percent. For example, the use of the term about 100 can refer to a range of from 90 to 110, inclusive. In instances in which the context requires otherwise and/or relative terminology is used in reference to something that does not include, or is not related to, a numerical value, the terms are given their ordinary meaning to one skilled in the art.

The above-detailed descriptions of embodiments of the technology are not intended to be exhaustive or to limit the technology to the precise form disclosed above. Although specific embodiments of, and examples for, the technology are described above for illustrative purposes, various equivalent modifications are possible within the scope of the technology as those skilled in the relevant art will recognize. For example, although steps are presented in a given order, alternative embodiments may perform steps in a different order. The various embodiments described herein may also be combined to provide further embodiments.

From the foregoing, it will be appreciated that specific embodiments of the technology have been described herein for purposes of illustration, but well-known structures and functions have not been shown or described in detail to avoid unnecessarily obscuring the description of the embodiments of the technology. Where the context permits, singular or plural terms may also include the plural or singular term, respectively.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with some embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

I/We claim:

1. A device for withdrawing bodily fluid from a subject, the device comprising:
a housing having a surface configured to be positioned against skin of the subject, the housing having an opening extending through the surface;
a flexible membrane stretched across the opening;
a floating island carried by the flexible membrane and adhesively coupled to the flexible membrane;
a skin-piercing assembly positioned at least partially within the housing and having a blade configured to pierce the skin of the subject; and
an actuator operably coupled to the skin-piercing assembly, wherein the actuator is movable relative to the housing to deploy the skin-piercing assembly through the opening and into the skin of the subject, and
wherein—
movement of the actuator away from the opening draws a vacuum within at least a portion of the housing, and
the skin of a patient is configured to be drawn at least partially through the opening in the housing and assume a two-tiered shape therein when the vacuum is drawn within the housing.

2. The device of claim 1 wherein all or substantially all of the surface of the housing comprises the opening.

3. The device of claim 1 wherein the opening has a vacuum diameter of about 30 mm.

4. The device of claim 1 wherein the floating island is a half-moon shape.

5. The device of claim 1 wherein the housing further comprises a one-way valve configured to allow gas to flow out of the housing and inhibit gas from flowing into the housing.

6. The device of claim 1 wherein the flexible membrane defines a precut hole aligned with the blade of the skin-piercing assembly.

7. The device of claim 1 wherein the floating island comprises walls configured to direct bodily fluid flow towards an outflow port of the housing.

8. The device of claim 1 wherein the housing further comprises a biasing member configured to bias the actuator and the skin-piercing assembly away from the opening.

9. The device of claim 1 wherein an inner surface of the housing is covered by a film bridge.

10. The device of claim 1 wherein the housing includes walls configured to direct bodily fluid flow towards an outflow port.

11. The device of claim 1 wherein the floating island includes features configured to deploy the blade of the skin-piercing assembly.

12. A device comprising:
a housing having a surface configured to be positioned against skin of a subject, the housing having an opening extending through the surface;
a flexible membrane stretched across the opening;
a floating island carried by the flexible membrane and adhesively coupled to the flexible membrane; and
a skin-piercing assembly positioned at least partially within the housing and having a blade configured to pierce the skin of the subject when deployed; and
wherein—
movement of the skin-piercing assembly away from the opening draws a vacuum within at least a portion of the housing, and
the skin of the subject is drawn at least partially through the opening in the housing and assumes a two-tiered shape therein when subject to the vacuum.

13. The device of claim 12 wherein the skin-piercing assembly includes one or more blades configured to make one or more cuts on the skin.

14. The device of claim 12 wherein the floating island is centered on the flexible membrane.

15. The device of claim 12 wherein the floating island includes flanges configured to direct fluid across the flexible membrane and towards an outflow port.

16. The device of claim 12 wherein a first tier of the two-tiered shape is larger than a second tier of the two-tiered shape.

17. A method of collecting blood from a patient, comprising:
placing a bodily fluid collection device on skin of the patient;
pressing an actuator of the bodily fluid collection device to deploy a blade to cut the patient's skin;
releasing the actuator to create a vacuum within the bodily fluid collection device to increase bodily fluid flow from a wound in the patient's skin;
collecting a desired sample in a collection reservoir; and
removing the bodily fluid collection device from the skin.

18. The method of claim 17 wherein pressing the actuator further comprises forcing air out of the bodily fluid collection device.

19. The method of claim 17 wherein releasing the actuator causes a biasing member of the bodily fluid collection device to bias the actuator away from the skin.

20. The method of claim 17 further comprising separating the collection reservoir from the bodily fluid collection device prior to removing the bodily fluid collection device from the skin.
